(19) United States
(12) Patent Application Publication
(10) Pub. No.: US 2023/0270691 A1
(43) Pub. Date: Aug. 31, 2023

(54) PHARMACEUTICAL COMPOSITIONS COMPRISING INSOLUBLE ACTIVE INGREDIENTS

(71) Applicant: Advent Therapeutics, Inc., Lumberville, PA (US)

(72) Inventors: Craig Alan Gelfand, Hancock, NH (US); Robert Segal, Gwynedd Valley, PA (US); David Lawrence Lopez, Lumberville, PA (US)

(21) Appl. No.: 18/007,589

(22) PCT Filed: Jun. 1, 2021
(86) PCT No.: PCT/US2021/035210

§ 371 Date: Dec. 1, 2022

Related U.S. Application Data
(60) Provisional application No. 63/069,927, filed on Aug. 25, 2020.
(60) Provisional application No. 63/054,425, filed on Jul. 21, 2020.
(60) Provisional application No. 63/032,768, filed on Jun. 1, 2020.

(51) Int. Cl. A61K 31/07; A61K 39/39; A61K 9/00; A61P 39/06; A61P 11/00; A61K 45/06

(52) U.S. Cl. CPC A61K 31/07; A61K 9/0078; A61K 39/39; A61K 45/06; A61P 11/00; A61P 39/06

(57) ABSTRACT

The present disclosure relates to pharmaceutical compositions suitable for inhalation, parenteral administration, and oral administration comprising a an insoluble active ingredient, pharmaceutical systems comprising them, methods of treatment and/or prophylaxis, and uses comprising them. The formulations, systems, and compositions provided by the present disclosure may be used and are useful in the treatment and/or prophylaxis of vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation- induced lung injury/damage and/or chronic lung injury/damage, including but not limited to delayed effects of acute radiations exposure (DEARS).

### STATEMENT OF GOVERNMENT SUPPORT

The inventions described herein were made, in part, with funds obtained from the National Heart Lung and Blood Institute, National Institutes of Health, Grant No. HL142353. The U.S. government may have certain rights in these inventions.

### CROSS-REFERENCE TO RELATED APPLICATIONS

This application claims the benefit of U.S. Provisional Pat. Application No. 63/069,927, filed Aug. 25, 2020, U.S. Provisional Pat. Application No. 63/054,425, Jul. 21, 2020, and U.S. Provisional Pat. Application No. 63/032,768, filed Jun. 1, 2020, the entire contents of which are incorporated herein by reference in their entirety.

### INCORPORATION BY REFERENCE

Each of the patent documents and references cited herein is incorporated by reference in their entirety.

### FIELD

This disclosure relates to pharmaceutical compositions comprising an insoluble active ingredient wherein the pharmaceutical composition is suitable for inhalation, parenteral administration, and/or oral administration. The disclosure also relates to pharmaceutical compositions comprising a therapeutically effective dose of an insoluble active ingredient, pharmaceutical systems comprising such pharmaceutical compositions, methods of treatment and/or prophylaxis of disease and/or disorders remedied by such pharmaceutical compositions, and uses of such pharmaceutical compositions. By way of non-limiting examples, the formulations, systems, and compositions disclosed herein may be used and are useful in the treatment and/or prophylaxis of vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS).

### BACKGROUND

### 1) Issues Relating to the Insolubility of Active Ingredients

Some active ingredients are insoluble in water but cells are surrounded by aqueous fluid. By way of a non-limiting example, the cells of the lung are in contact with aqueous fluid. Without being limited to a particular theory, it is believed that an active ingredient must dissolve in the fluid before being able to interact biochemically with a cell surface . This causes a problem for insoluble active ingredients, including by not limited to such ingredients intended to be delivered by inhalation. See Tolman, Justin A., and Robert O. Williams III. “Advances in the pulmonary delivery of poorly water-soluble drugs: influence of solubilization on pharmacokinetic properties.” Drug development and industrial pharmacy 36.1 (2010): 1-30. There are well-known challenges for the effective solubilization of otherwise insoluble active ingredients for administration to patients, including but not limited to administration to a subject’s lung tissue, and particularly for situations where inhalation of the drug may have potential therapeutic benefits both to the lung tissue and systemically.

### 2) Vitamin A and Conditions of Prematurity

Premature births and associated complications continue as a major public health care problem. Advances in intervention, treatment modalities, and medical devices consistently increase survival rates of extremely premature births, toward ever younger post-menstrual age (PMA) births, leading to the inevitable consequence of increasing incidence of common sequelae of prematurity. Bronchopulmonary dysplasia (BPD), also known as chronic lung disease (CLD) of infants/infancy, is among these conditions, defined historically as the need for oxygen supplementation and/or ventilatory support at 28 days of age and/or per the current NIH definition as a need for supplemental oxygen and/or ventilatory support through and at 36 weeks PMA (see, for example, Ehrenkranz RA, Walsh MC, Vohr BR, et al. Validation of the National Institutes of Health Consensus Definition of Bronchopulmonary Dysplasia. Pediatrics 2005 Dec 1;116(6):1353-60 PMID: 16322158). Each year, 10,000-15,000 premature infants develop BPD (see, for example, Bronchopulmonary Dysplasia, National Heart, Lung and Blood Institute (NHLBI) [Internet] [cited 2019 Apr 2] https://www,nhlbi.nih.gov/health-topics/bronchopulmonary-dylplasia, and Strueby, L., Thebaud, B., Advances in bronchopulmonary dysplasia, Expert Rev Respir Med, 2014 June 8(3); 327-38 PMID: 24666156), and rates of BPD in infants of 22-28 weeks PMA have been slowly increasing despite more frequent use of non-invasive ventilatory support and other neonatology practice improvements (see, for example, Stoll BJ et al., Trends in Care Practices, Morbidity, and Mortality of Extreme Preterm Neonates, 1993-2012. JAMA. 2015 Sep 8;314(10):1039-1051 PMCID: PMC4787615). BPD treatment often includes prolonged mechanical ventilation or oxygen support and/or recurrent hospitalizations for pulmonary infections and other complications of disrupted alveolarization (Baker, C.D., Alvira, C.M. Disrupted lung development and bronchopulmonary dysplasia: opportunities for lung repair and regeneration, Curr Opin Pediatr. 2014, June 26(3): 306-14. PMCID: PMC4121955). Contrary to previous belief that compensatory lung growth occurs with age (see, for example, O′Reilly, M., Sozo, F., Harding, R., Impact of preterm birth and bronchopulmonary dysplasia on the developing lung: long-term consequences for respiratory health, Clin Exp Pharmacol Physiol., 2013, November 40(11), 765-73. PMID: 23414429), evidence now suggests that even mild BPD cases continue to show impaired lung function in childhood and beyond (see, for example, Greenough A, et al., Lung volumes in infants who had mild to moderate bronchopulmonary dysplasia. Eur J Pediatr. 2005 Sep; 164(9):583-586 PMID: 15937699). Impaired lung function from BPD persists into adulthood, contributing to chronic respiratory diseases; neonatal BPD strongly predicts lifelong abnormal lung function ((see, for example, Ehrenkranz RA, Walsh MC, Vohr BR, et al. Validation of the National Institutes of Health Consensus Definition of Bronchopulmonary Dysplasia. Pediatrics 2005 Dec 1;116(6):1353-60 PMID: 16322158; and Wong PM, Lees AN, Louw J, et al. Emphysema in young adult survivors of moderate-to-severe bronchopulmonary dysplasia. Eur Respir J 2008 Aug;32(2):321-8 PMID:18385172). Moreover, infants with BPD suffer impaired physical growth, neurocognitive delays, and cardiac dysfunction including pulmonary hypertension (see, for example, Cerny L, Torday JS, Rehan VK. Prevention and treatment of bronchopulmonary dysplasia: contemporary status and future outlook. Lung 2008 Apr; 186(2):75-89 PMID: 18228098, and Levy PT, Dioneda B, Holland MR, et al. Right ventricular function in preterm and term neonates: reference values for right ventricle areas and fractional area of change. J Am Soc Echocardiogr 2015 May;28(5):559-69 PMCID: PMC4532398). The cost of BPD care further exacerbates current concerns. In 2012, H-CUPnet (HCUPnet [Internet]. HCUPnew. [cited 2017 Sep 2] Available from https://hcupnet.ahrq.gov) reported an average hospital charge of $264,350 for treating a single BPD case and a mean stay of 32.6 days, and in 2016 Bhandari et al. (Bhandari A., et al. BPD Following Preterm Birth: A Model for Chronic Lung Disease and a Substrate for ARDS in Childhood. Front Pediatr. 2016;4:60. PMCID: PMC4909128) wrote “The overall costs of treating babies with BPD in the US are estimated to be $2.4 billion”. Better methods to prevent BPD in an expanding neonatal patient population will benefit families and payors alike.

Preterm infants are expected to be deficient in critical metabolites, including vitamin A (vitA). The human fetus accumulates vitamin A primarily in the 3rd trimester of pregnancy. Premature infants have reduced hepatic stores of retinoids (Mactier H, Weaver LT. Vitamin A and preterm infants: what we know, what we don’t know, and what we need to know. Archives of Disease in Childhood — Fetal and Neonatal Edition 2005 Mar 1;90(2): F103-8. PMID: 15724031). In plasma, vitamin A is complexed to retinol-binding protein (RBP), which is further complexed with transthyretin (Mactier H, Weaver LT. Vitamin A and preterm infants: what we know, what we don’t know, and what we need to know. Archives of Disease in Childhood — Fetal and Neonatal Edition 2005 Mar 1;90(2): F103-8. PMID: 15724031). Premature infants have lower concentrations of plasma RBP than term infants, and most preterm infants have both low plasma vitamin A concentrations and low plasma retinol/RBP molar ratios (vitamin A plasma concentrations below 100 µg/L indicate severe deficiency and depleted liver stores) (Shenai JP. Viamin A supplementation in very low birth weight neonate: rationale and evidence. Pediatrics 1999 Dec:104(6): 1369-1374, PMID: 10585990; Greene HL, Phillips BL, Franck L, et al. Persistently low blood retinol levels during and after parenteral feeding of very low birth weight infants: examination of losses into intravenous administration sets and a method of prevention by addition to a lipid emulsion. Pediatrics 1987 Jun;79(6):894-900. PMID: 3108847; and Shenai JP, Rush MG, Stahlman MT, Chytil F. Plasma retinol-binding protein response to vitamin A administration in infants susceptible to bronchopulmonary dysplasia. J Pediatr 1990 Apr; 116(4):607-14. PMID:218233). Plasma RBP response and the relative rise in plasma retinol concentration following IM vitamin A administration are useful assays of functional vitA status (Zachman RD, Samuels DP, Brand JM, Winston JF, Pi JT. Use of the intramuscular relative-dose-response test to predict bronchopulmonary dysplasia in premature infants. Am J Clin Nutr 1996 Jan;63(1):123-9. PMID: 8604659).

In some cases, term infants can also have such deficiencies, including a deficiency in vitamin A, due to malnutrition or vitamin A deficiency of the mother, especially during gestation, or caused by a genetic deficiency or other inherent disease of the mother or the baby.

Vitamin A has been shown to play a critical role in lung development, and the medical condition of vitamin-A-deficiency (VAD) has been posited to predispose or contribute to development of BPD (Chytil F. The lungs and vitamin A. Am J Physiol 1992 May;262(5 Pt 1): L517-527. PMID:1317113; Shenai JP, Chytil F, Parker RA, Stahlman MT. Vitamin A status and airway infection in mechanically ventilated very-low-birth-weight neonates. Pediatr Pulmonol 1995 May; 19(5):256-61. PMID7567199; Hustead VA, Gutcher GR, Anderson SA, Zachman RD. Relationship of vitamin A (retinol) status to lung disease in the preterm infant. J Pediatr 1984 Oct;105(4):610-5. PMID6481538; and Shenai JP, Chytil F, Stahlman MT. Vitamin A status of neonates with bronchopulmonary dysplasia. Pediatr Res 1985 Feb;19(2):185-8 PMID: 3982875). In earlier studies, very low birth-weight infants who developed BPD have lower vitamin A levels than similar infants without BPD (Hustead VA, Gutcher GR, Anderson SA, Zachman RD. Relationship of vitamin A (retinol) status to lung disease in the preterm infant. J Pediatr 1984 Oct;105(4):610-5. PMID6481538; and Shenai JP, Chytil F, Stahlman MT. Vitamin A status of neonates with bronchopulmonary dysplasia. Pediatr Res 1985 Feb; 19(2): 185-8 PMID: 3982875). Preclinical studies show that low plasma and tissue vitamin A levels contribute to pulmonary histopathological changes consistent with BPD in preterm infants (Lancillotti F, Darwiche N, Celli G, De Luca LM. Retinoid status and the control of keratin expression and adhesion during the histogenesis of squamous metaplasia of tracheal epithelium. Cancer Res 1992 Nov 15;52(22):6144-6152. PMID: 1384955; and Baybutt RC, Hu L, Molteni A. Vitamin A deficiency injures lung and liver parenchyma and impairs function of rat type II pneumocytes. J Nutr 2000 May;130(5):1159-65. PMID: 10801913), and can be reversed by restoring adequate vitamin A status (Hind M, Maden M. Retinoic acid induces alveolar regeneration in the adult mouse lung. Eur Respir J 2004 Jan;23(1):20-7. PMID14738226). Similar changes are observed in ventilated infants with chronic neonatal lung injury, and who are vitamin A deficient (Hustead VA, Gutcher GR, Anderson SA, Zachman RD. Relationship of vitamin A (retinol) status to lung disease in the preterm infant. J Pediatr 1984 Oct;105(4):610-5. PMID6481538).

Studies have shown that vitamin A supplementation facilitates recovery from lung injury, accelerates alveolar morphogenesis and has been shown to reduce the incidence of BPD in preterm infants (Guimarães H, Guedes MB, Rocha G, Tomé T, Albino-Teixeira A. Vitamin A in prevention of bronchopulmonary dysplasia. Curr Pharm Des 2012;18(21):3101-3113. PMID 22564302; Tropea K, Christou H. Current pharmacologic approaches for prevention and treatment of bronchopulmonary dysplasia. Int J Pediatr 2012;2012:598-606. PMICD: PMC3259479; and Young TE. Nutritional support and bronchopulmonary dysplasia. Journal of Perinatology 2007;27:S75-S78). There is compelling evidence to support that vitamin A supplementation can both prevent BPD and treat the underlying progressive disease processes that begin within hours to days after birth that lead to clinical manifestations of BPD. Despite such information, vitamin A dosing for BPD is not commonplace. Commercially available vitamin A for parenteral administration is only FDA-approved to treat VAD in preterm infants and not specifically approved for prevention of BPD. Oral dosing has proven insufficient for VAD supplementation, presumably because preterm neonates, especially very low birthweight infants, tend to be intolerant to enteral feeding and/or their immature gut development cannot adequately support absorption of vitA (Rush MG, Shenal JP, Parker RA, Chytil F. Intramuscular versus enteral vitamin A supplementation in very low birth weight neonates. The Journal of Pediatrics 1994;125(3):458-62. PMID: 8071758). A high-dose oral vitA regimen is under investigation [the NeoVitA clinical trial; EudraCT No. 2013-001998-24] but outcomes data are not yet available. In clinical practice, total parenteral nutrition (TPN) is typically provided to preterm infants who are unable to tolerate oral feeds, however, TPN with vitamin A also has limitations in providing sufficient vitamin A to prevent BPD (Greene HL, Phillips BL, Franck L, et al. Persistently low blood retinol levels during and after parenteral feeding of very low birth weight infants: examination of losses into intravenous administration sets and a method of prevention by addition to a lipid emulsion. Pediatrics Jun; 1987;79(6):894-900. PMID: 3108847).

A series of studies have evaluated vitamin A dosed by intramuscular injection (IM) for prevention and treatment of BPD, including a well-designed NICHD sponsored clinical trial (Tyson JE, Wright LL, Oh W, et al. Vitamin A Supplementation for Extremely-Low-Birth-Weight Infants. New England Journal of Medicine 1999;340(25):1962-8. PMID: 10379020), and summarized by a Cochrane Review (Darlow BA, Graham PJ, Rojas-Reyes MX. Vitamin A supplementation to prevent mortality and short- and long-term morbidity in very low birth weight infants. ICochrane Database of Systematic Reviews [Internet]. John Wiley & Sons, Ltd; 2016. Available from: http://onlinelibrary.wiley.com/doi/10.1002/14651858.CD000501..pub4/abstract) which describes a number-needed-to-treat (NNT) of about 14. This number can either be viewed as favorable (in light of BPD frequency and treatment costs, per above) or perhaps as disappointing in that there remains a clear need for a lower-NNT treatment to be discovered. Utility for parenteral vitamin A for BPD has been recently reinforced in a Neonatal Research Network (NRN) review and a separate analysis revalidating the cost/benefit of IM vitamin A therapy (Couroucli XI, Placencia JL, Cates LA, Suresh GK. Should we still use vitamin A to prevent bronchopulmonary dysplasia? J Perinatol 2016;36(8):581-585. PMID: 27228508). Yet parenteral vitamin A continues to face major impediments to broad utilization, likely exacerbated by concerns related to repeated invasive IM dosing per the current NICU regimen (12 injections over 4 weeks), especially in preterm neonates because of limited muscle mass, fragile skin, increased bleeding susceptibility, and pain (Green, J., et al., It’s agony for us as well: Neonatal nurses reflect on iatrogenic pain. Nurs Ethics. 2016:23(2):176-90. PMID: 254887861; and Services D of H&H. Intramuscular injections for neonates [Internet] Available from: Https://www2.health.vic.gov.au:443/hospitals-and-health-services/patient-care/perinatal-reproductive/neonatal-handbook/procesures/intramuscular-injections).

Taylor (Sneha K. Taylor et al., Inhaled Vitamin D: A Novel Strategy to Enhance Neonatal Lung Maturation, 194 LUNG 931-943 (2016)) discloses an inhaled vitamin D formulation. However, despite vitamin D being a fat-soluble vitamin, Taylor does not disclose a formulation of vitamin D with a surfactant nor suggests any advantages stemming from the presence of a surfactant in the formulation. Before the present disclosure, it would not have been appropriate to merely exchange one vitamin for another in a formulation and expect similar outcomes.

Biesalski (Hans Biesalski et al. Retinyl palmitate supplementation by inhalation of an aerosol improves vitamin A status of preschool children in Gondar (Ethiopia), 82 B_{R}. J. N_{UTR}. 179-82 (1999)) discloses an inhalable form of a vitamin A formulation. However, Biesalski’s formulation does not contain a surfactant nor does Biesalski suggest any advantages stemming from the presence of a surfactant in the formulation. More generally, Biesalski’s formulation does not include any provision for water-miscibility of the vitamin A formulation, such that any vitamin A delivered by Biesalski’s method is likely insoluble in the fluids of the lung, unlike the pharmaceutical compositions of the current disclosure. As such, Biesalski’s formulations may be of diminished or potentially no direct biochemical effect upon the lung. This is consistent with the limited data set reported in Biesalski that, notably, does not include any data describing changes in lung tissue status or lung health pursuant to dosing.

Bockow (U.S. Pat. No. 5,411,988) discloses a composition with omega-3 and/or omega-6 fatty acids, vitamin A, and a nonionic surfactant. While Bockow discloses that composition “may also be applied directly to the lung . . .,” there is no disclosure in Bockow that the composition be in aerosol form.

Accordingly, there is a long felt but unmet need for the effective provision of vitamin A to neonates that avoid the deficiencies of the approaches described above. To that end, inhalable compositions of vitamin A suitable for direct delivery to the lungs are disclosed, which have unexpectedly been found to offer enhanced BPD prophylaxis versus intramuscular (IM) injection.

### 3) Insoluble Active Ingredients to Avoid Long-Term Lung Damage Associated With Hyperoxia and Other Injury

“Hyperoxia” or “hyperoxic conditions” occur when humans or animals are exposed to oxygen levels in excess of normal conditions. For humans and animals that normally respire atmospheric air (normal environmental air), the normal amount of oxygen (or ‘normoxia’ conditions) is typically 20.8-21.0% oxygen. Hyperoxia occurs when oxygen exceeds those levels.

The most common reason for humans being exposed to hyperoxia is medical need: hyperoxic conditions may be intentionally introduced to help patients suffering from impaired pulmonary function including but not limited to situations where a patient is not achieving sufficient blood oxygen levels. Low blood oxygen levels are a life-threatening condition requiring immediate intervention. Introducing such patients to oxygen levels up to about 30% oxygen in inspired gas is common in hospital or emergency room settings for treating pulmonary insufficiency.

Lung damage may also be associated with other pathways, including but not limited to vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections, and/or viral infections. Lung damage may also be caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS), and chemical, biological and/or radio-nuclear injury. (See, for example, Morgan GW & Breit SN “Radiation and the lung: a reevaluation of the mechanisms mediating pulmonary injury” Int J Radiat Oncol Biol Phys 1995, 31, (2), 361-9; Movsas, B et al., “Pulmonary radiation injury” Chest 1997, 111, (4), 1061-76; Tsoutsou PG & Koukourakis MI, “Radiation pneumonitis and fibrosis: mechanisms underlying its pathogenesis and implications for future research” Int J Radiat Oncol Biol Phys. 2006;66:1281-93.).

Chemical agents can also induce lung damage that can lead to pulmonary insufficiency and the medical need to induce hyperoxia. These agents include but are not limited to chemical and biological agents, exposure to radiation or radionuclides, caustic agents, volatile chemicals and the like. Such chemicals may be associated with warfare but can also include workplace injuries, smoke inhalation, industrial accidents, or be from natural sources like volcanoes.

Biological causes of reduced pulmonary function and the need for medical hyperoxia include but are not limited to infections. By way of non-limiting examples, infections of the lung include neonatal sepsis, hospital-acquired sepsis, sepsis from premature rupture of membranes, and pneumonia. Other non-limiting examples include measles, meningitis, necrotizing enterocolitis, other viral infections such as those associated with influenza virus, SARS-CoV (the virus associated with severe acute respiratory syndrome), MERS-CoV (the virus associated with Middle East respiratory syndrome), SARS-CoV2 (the virus associated with COVID-19) and related viridae, or other bacterial infections. Therapeutic hyperoxia can also be used routinely in ambulatory patients with chronic pulmonary conditions such as chronic obstructive pulmonary disease (COPD) or cystic fibrosis. The increasing ubiquity of commercially available portable oxygen concentrators serves as an example of how commonplace this sort of hyperoxia exposure is becoming.

Oxygen itself is among the chemicals that can damage the lung, bringing risk along with the known benefits in supporting blood oxygenation. Exposure to hyperoxia increases the generation of particular reactive oxygen species that will increase in the lung cells. These ultimately can lead to cell apoptosis and tissue necrosis (Dias-Freitas et al., “Molecular mechanisms underlying hyperoxia acute lung injury.” Respir Med (2016 Oct);119:23-28), so hyperoxia as an intervention is always approached carefully. Longer exposure or higher oxygen levels increases the risk. Any damaged induced by oxygen can ultimately exacerbate the original condition that led to the need for hyperoxia. Medical use of increased oxygen therefore is a common mode of iatrogenic damage; such damage can be defined as “induced inadvertently by a physician, surgeon, or other medical professional or by medical treatment or diagnostic procedure.” This is also why oxygen beyond 30-40% is rarely used, as the damage induced to lung tissue can far outweigh the benefits, even in cases of acute pulmonary insufficiency. In this regard, there is a long-felt but unmet need for a therapy that can reduce oxygen-induced damage to lung tissue as such a therapy could allow for increased concentrations of oxygen.

“Hyperoxic damage” can be damage to lung tissue caused by exposure to oxygen in excess of that found in ambient conditions. Therapeutic hyperoxia, particularly at higher levels, can lead to the condition known as Acute Lung Injury (ALI) (see, e.g., Dias-Freitas et al.) which, if unmitigated, can progress to Acute Respiratory Distress Syndrome (ARDS). These acute conditions that also bring risk of chronic complications and morbidities. One clear effect is that acute lung tissue damage reduces the overall amount of lung function - there is less healthy tissue to support normal lung function. The damage to cells can induce apoptosis and/or tissue necrosis. This can be an expanding threat to healthy tissue. Acute lung tissue damage can also include damage to local vasculature, allowing blood to leak into the normally air-exposed lung lumen that can potentially be sufficient to result in dangerous clot formation. Chemotactic signals that recruit necessary mediators of tissue repair, e.g. neutrophils, can also induce localized inflammation, an intrinsic feature of the body’s repair response but that also can further exacerbate oxygen insufficiency. Chronic tissue damage is also a concern, especially as seen with DEARS and also SARS-CoV-2, as non-limiting examples, particularly scarring and fibrosis that can result from tissue healing as mentioned above: if previously healthy lung tissue is replaced with scarring/fibrosis, the surface area for gas exchange is reduced, essentially permanently. An ideal intervention would support or even enhance the necessary biochemical repair processes, while at the same time reducing deleterious natural responses like excessive inflammation and eliminating the scarring and fibrosis underlying subsequent chronic health issues.

The broad scope of reasons for which medically-induced hyperoxia is indicated leaves a long-felt but unmet need to address the associated risks, morbidities. and even mortality associated with iatrogenic damage caused by this critical intervention. Avoiding long-term pulmonary complications caused by medical hyperoxia from, by way of non-limiting examples, emergency interventions, lung infection, or chemically/biologically induced lung damage will bring significant societal benefit.

The nature of hyperoxic lung damage in premature babies has some similarities but also some key differences when compared to hyperoxia effects on developed lung.

These differences can make BPD harder to treat than hyperoxia-related issues in more developed lung tissue, such as those of children older than neonates and adults. As such, prematurely-born neonates represent a unique situation, as an obligatory outcome of premature delivery such that lungs are immediately exposed to hyperoxia conditions. This is because in the womb, normal healthy gestation is in the absence of any air exposure, so in-utero ‘normoxia’ is zero. For a normal full-term or nearly full-term birth, the first oxygen exposure to room air would be typical normoxia conditions, with the lungs developed and ready for proper respiratory function. The lungs of prematurely born babies are not sufficiently developed for any air exposure, so even room air is ‘hyperoxic’ and can initiate oxygen-mediated lung damage (Tin W, Gupta S. Optimum oxygen therapy in preterm babies. Arch Dis Child Fetal Neonatal Ed. 2007 Mar;92(2):F143-7). The earlier a premature birth occurs, the higher the risk of hyperoxic lung damage. Additionally, earlier births will trend toward experiencing more severe damage. The acute timeframe also is unique from adult hyperoxia, in that premature lung is still developing, so the nature of “hyperoxic damage” in prematurity needs to include the recognition that normal lung tissue development, for example normal alveolarization that is already disrupted by premature birth can be further disrupted by hyperoxia damage. An ideal BPD-preventative would support normal lung development during the acute period between premature and what would have been “term” birth. In addition, such a preventative would support any necessary repair of oxygen-induced lung tissue damage. This would be similar to the adult/developed lung situation. As disclosed above, impaired lung function from BPD can persist long after hyperoxia. In this chronic phase, the resulting lung damage has similarities to the adult situation, with resulting scarring leading to lifelong pulmonary complications. However, with BPD there is the potential for unique exacerbation of hyperoxia-related damage due to the prior disruption of normal third-trimester alveolar development.

In summary, it is well known that hyperoxia for any patient of any age can damage lung tissue, representing both acute threat and high risk for chronic morbidity. Therefore, a therapy or prophylactic against hyperoxia lung damage might be relevant to patients of any age, including premature neonates, children older than neonates and adults. And there is a long-felt but unmet need for such therapies.

There is similarly a long-felt but unmet need for therapies to remedy vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS).

### 4) Insoluble Active Ingredients and Infection

Bacterial infection in humans and animals is a common condition, often requiring medical intervention. Most infections begin locally before spreading to other organs. Typical initial infections and their effects are related to the mechanism of entry of the infectious entity. Inhalation will typically first infect the respiratory pathway, for example including nasal passages, especially the sinus cavities, throat, various structures of the lung, including the bronchi or alveoli, or possibly the oral cavity. Ingestion can lead to infection of the digestive tract, affecting anything along that pathway including, for example, the oral cavity, the esophagus, the stomach or the intestines. Skin lesions can lead to localized dermal or subdermal infections. Beyond skin, any outside-facing tissue or organ can represent an entry point or surface subject to infection, such as eyes, ears, urinary tract, etc. Any infectious agent entering through any of these routes can migrate beyond the initial point of entry or localized infection site, with the potential of initiating infection in virtually any other organ, especially if the agent becomes bloodborne. If unchecked, especially bloodborne pathogen can induce systemic sepsis, which can be a life-threatening condition.

Infectious agents typically are bacteria, viruses, or animal organisms. Single-celled (or similarly single-particle) entities overall represent immense biological diversity. They can be summarized into a few general categories. Animal, eukaryotic, infectious agents can be single-celled, with yeasts being a common cause of infection, among many other organism phyla. Bacteria, prokaryotes, are typically segregated into two categories, gram-positive and gram-negative, depending on the structure of their external coats / cell walls. Infectious bacteria typically replicate on their own, relying on the host that they have infected for nutrition and a satisfactory growth environment. Some bacteria, such as the mycobacteria class that includes M. tuberculosis, can replicate, under certain conditions, inside of the very immune cells that engulf them to ‘kill’ them (phagocytosis) as an early stage in the host’s response to infection. Viruses, by contrast, rely on biochemical processes of the host to replicate, and therefor require intracellular access, often for their entire replication cycle. Influenza and SARS-related viridae are examples of viruses that have predilection for infecting the airways including the lung, producing both acute and chronic sequelae. Virus infections can spread through dissemination of mature viral particles throughout the extracellular milieu of infected organs or organisms, including through the bloodstream, or intracellularly through host-cell replication itself, essentially piggy-backing along with normal host cell replication processes.

Vitamin A is a vital biochemical, known to be involved in a wide variety of processes during all stages of life. Vitamin A status has been shown to be a critical factor in infection, across a diverse range of issues including elevated risk of infection due to low vitamin A levels, the ability to mount effective biological responses to an active infection, and in building an appropriate immunological response to vaccination against infection. Vitamin A, including the majority of the pharmaceutically acceptable isomers, analogs, and/or derivatives thereof, are essentially insoluble in aqueous solutions by themselves.

Vitamin A deficiency (often referred to as VAD) is a condition in which vitamin A levels are lower than found in a normal healthy condition. VAD can be caused by a variety of conditions. The most common cause is dietary insufficiency, treated by supplements or by increasing intake of food rich in vitamin A or vitamin A precursors. VAD can also be caused by insufficiency in vitamin A uptake from the digestive system, with underlying causes being genetic defect or the result of surgery, often for bariatric intervention, after emergency trauma, or resections for cancer or other disease, leading to absence of the proper biochemical mechanisms to absorb vitamin A or related precursors from food. In these cases, parenteral supplements become necessary, currently requiring injection of appropriate vitamin A formulation. Infants can become vitamin A deficient if the mother is vitamin A deficient. Premature birth can result in neonatal VAD, as the baby is removed early from the normal umbilical supply of nutrients, with the severity of the condition tending to increase with the extent of prematurity. Premature infants with VAD are subject to critical developmental deficiency, particularly of the lung, eye and brain, and available treatment includes injected and/or oral supplementation (Jon E. Tyson et al., Vitamin A Supplementation for Extremely-Low-Birth-Weight Infants, 340 NEW ENGLAND JOURNAL OF MEDICINE 1962-1968 (1999); Vitamin A supplementation to prevent mortality and short- and long-term morbidity in very low birth weight infants, in COCHRANE DATABASE OF SYSTEMATIC REVIEWS, http://onlinelibrary.wiley.com/doi/10.1002/14651858.CD000501.pub4/abstract.)

Vitamin A deficiency is also associated with an increased risk of infection. This is a well-known phenomenon, drawing from the early days of medicinal biochemistry research. In 1923, Werkman reported that lack of vitamins was observed to increase risk of bacterial infection in various laboratory animals (C. H. Werkman, Immunologic Significance of Vitamins: //. Influence of Lack of Vitamins on Resistance of Rat, Rabbit and Pigeon to Bacterial Infection, 32 THE JOURNAL OF INFECTIOUS DISEASES 255-262 (1923)). In that same year, Daniels et al reported that “A diet lacking in fat soluble [vitamin] A makes possible the bacterial invasion of the mucous membranes of the ear and nasal cavities” (Amy L. Daniels et al., NASAL SINUSITIS PRODUCED BY DIETS DEFICIENT IN FAT-SOLUBLE A VITAMIN, 81 JOURNAL OF THE AMERICAN MEDICAL ASSOCIATION 828-829 (1923)).

Increased susceptibility to respiratory pathway infection, from nasal to lung, due to VAD has continued to be a common topic in medicine up to the present day. For example, a 2020 report shows that severe pneumonia, via mycoplasma pneumoniae infection, is more often seen in vitamin-A-deficient children (Yan Xing et al., Vitamin A deficiency is associated with severe Mycoplasma pneumoniae pneumonia in children, 8 ANNALS OF TRANSLATIONAL MEDICINE (2020), https://www.ncbi.nlm.nih.gov/pmc/articles/PMC7049042/ (last visited Jul. 9, 2020)); this is but one among a score of published reports of the link between VAD and infection risk. At a more detailed level, the effects of VAD have been correlated to delayed ability to diminish pathogen count by normal immune system mechanisms; examples are diverse, including Sendai virus (a.k.a. murine parainfluenza, a respirovirus) levels in nasal passages (Rhiannon R. Penkert et al., Vitamin A deficient mice exhibit increased viral antigens and enhanced cytokine/chemokine production in nasal tissues following respiratory virus infection despite the presence of FoxP3 + T cells, 28 INTERNATIONAL IMMUNOLOGY 139-152 (2016)) and Citrobacter rodentium (bacterial) levels in the gut (Katherine H. Restori et al., Streptococcus pneumoniae-Induced Pneumonia and Citrobacter rodentium-Induced Gut Infection Differentially Alter Vitamin A Concentrations in the Lung and Liver of Mice12, 144 THE JOURNAL OF NUTRITION 392-398 (2014)).

The underlying tendency toward infection during VAD has been postulated to be due, at least in part, to locally altered microbiota of the affected organ. In recent studies, altered innate bacterial content of the lung (Vitamin A Deficiency and the Lung Microbiome, in D70. REVISITING LUNG INFECTION AND INFLAMMATORY AXES A7442-A7442, https://www.atsjournals.org/doi/abs/10.1164/ajrccm-conference.2020.201.1_MeetingAbstracts.A7442 (last visited Jul. 9, 2020)) and gut (Namrata lyer & Shipra Vaishnava, Vitamin A at the interface of host-commensal-pathogen interactions, 15 PLOS PATHOGENS (2019), https://www.ncbi.nlm.nih.gov/pmc/articles/PMC6553882)) have been linked to VAD, with assertion that the altered microbiome creates an environment for pathogen infection to take hold.

Another aspect of immune system disfunction associated with VAD is insufficient response to vaccines. For example, neonatal calves with VAD failed to mount an immune response to a vaccine to respiratory syncytial virus (Jodi L. McGill et al., Vitamin A deficiency impairs the immune response to intranasal vaccination and RSV infection in neonatal calves, 9 SCIENTIFIC REPORTS (2019), https://www.ncbi.nlm.nih.gov/pmc/articles/PMC6805856/ (last visited Jul. 9, 2020)). Lower immunoglobulin levels in calves were also reported after exposure to inactivated bovine coronavirus, versus a stronger response in vitamin-A-sufficient animals (Junbae Jee et al., Effects of dietary vitamin A content on antibody responses of feedlot calves inoculated intramuscularly with an inactivated bovine coronavirus vaccine, 74 AMERICAN JOURNAL _{OF} VETERINARY RESEARCH 1353-1362 (2013)). This is particularly relevant in view of the ongoing pandemic in connection with SARS-CoV-2 and related viridae and similarly to regarding influenza and related viridae, where vaccination is seen as critical for population health. Non-limiting examples of other respiratory viruses include enteroviruses, rhinoviruses, influenza viruses type A, influenza viruses type B, respiratory syncytial viruses, adenoviruses, and Epstein-Barr viruses.

Vitamin A levels have also been correlated to the extent of immune response to vaccination. Vitamin A doses to dietarily induced VAD suppresses vaccine efficacy, and that vitamin A supplementation improves vaccine-induced antibody titers, for pneumonia (Rhiannon R. Penkert et al., Influences of Vitamin A on Vaccine Immunogenicity and Efficacy, 10 FRONTIERS IN IMMUNOLOGY (2019), https://www.ncbi.nlm.nih.gov/pmc/articles/PMC6651517/ (last visited Jul. 9, 2020)) and influenza (S. L. Surman et al., Vitamin Supplementation at the Time of Immunization with a Cold-Adapted Influenza Virus Vaccine Corrects Poor Mucosal Antibody Responses in Mice Deficient for Vitamins A and D, 23 CLINICAL _{AND} VACCINE IMMUNOLOGY : CVI 219-227 (2016)) vaccinations in mice and coronavirus vaccinations in bovines (Jee et al.). Vitamin A doses have been shown to stimulate various attributes of immune system activity, including higher innate antibody levels during active pneumonia infections (P. Zhang et al., Low-dose vitamin A therapy on T lymphocyte function in neonatal pneumonia, 22 EUROPEAN REVIEW FOR MEDICAL AND PHARMACOLOGICAL SCIENCES 4371-4374 (2018)), immune memory as evidenced by increases in vaccine-specific central memory-like CD8⁺ T cells (Zhiyi Huang et al., Role of Vitamin A in the Immune System, 7 JOURNAL _{OF} CLINICAL MEDICINE (2018), https://www.ncbi.nlm.nih.gov/pmc/articles/PMC6162863/ (last visited Jul. 9, 2020)), and increased resistance to radiation-induced pneumonitis (C. A. Redlich et al., Vitamin A inhibits radiation-induced pneumonitis in rats, 128 THE JOURNAL OF NUTRITION 1661-1664 (1998)). These reports demonstrate both an immediate benefit, such as evidence of increased levels of immunoglobulins systemically and at the site of the infection, and longer-term benefit with lower residual immune activity weeks after clearing the acute infection suggestive of more robust initial clearance. Such observations suggest one mechanism underlying the observations (described above) that VAD increases risk of infection, suggesting that vitamin A plays a key role in supporting immune response to infection. Similarly, maternal supply of vitamin A during gestation is key to establishing a healthy lymphoid system and ‘immune structures’ in offspring (Serge A. van de Pavert et al., Maternal retinoids control type 3 innate lymphoid cells and set the offspring immunity, 508 NATURE 123-127 (2014)).

Vitamin A stores are mobilized as part of a systemic response to infection, as reflected by changes in vitamin A levels both at the site of infection and systemically. For example, a 2020 publication describes that rats exposed to streptococcal pneumonia showed reduced levels of vitamin A in their serum and lung tissue (Yonglu Tian et al., Vitamin A supplement after neonatal Streptococcus pneumoniae pneumonia inhibits the progression of experimental asthma by altering CD4+T cell subsets, 10 SCIENTIFIC REPORTS (2020), https://www.ncbi.nlm.nih.gov/pmc/articles/PMC7060180/ (last visited Jul. 9, 2020)), with the depleted lung level showing persistence after the infection was cleared. A separate study with rodents found the same results under either lung or gut infection (Restori et al.), with reduced vitamin A level in lung, gut and serum reported. Infection with a bronchitis-inducing coronavirus in chickens was shown to reduce blood vitamin A level (C. E. West et al., Epithelia-damaging virus infections affect vitamin A status in chickens, 122 THE JOURNAL OF NUTRITION 333-339 (1992)). Notably, in the latter two studies, and also in (lyer & Vaishnava), the liver-synthesized chaperone proteins that solubilize vitamin A in the blood, specifically retinol binding protein and transthyretin, are also reduced in the blood during infection. Conversely, it has also been reported that vitamin A rises after recovery from infection (Rosangela da Silva et al., [Plasma vitamin A levels in deprived children with pneumonia during the acute phase and after recovery], 81 JORNAL DE PEDIATRIA 162-168 (2005)).

Vitamin A doses have also been shown to yield overall treatment and health improvements during and after acute infection. A study of pneumonia in children found that oral vitamin A supplementation produced tangible medical benefits including earlier reduction in fever and, importantly, that the first line antibiotic treatment was 29% more likely to be effective (L. C. Nacul et al., Randomised, double blind, placebo controlled clinical trial of efficacy of vitamin A treatment in non-measles childhood pneumonia., 315 BMJ : BRITISH MEDICAL JOURNAL 505-510 (1997)). Vitamin A dosing helps in clearing virus earlier upon acute infection, a benefit in reducing the extended periods of cytokine elevation (Penkert et al.) and immune cell activity (R. R. Penkert et al., Vitamin A deficient mice exhibit increased viral antigens and enhanced cytokine/chemokine production in nasal tissues following respiratory virus infection despite the presence of FoxP3+ T cells. Int Immunol. 2016) which have deleterious longer-term systemic effects.

Other mechanisms elicited by vitamin A and related compounds have been attributed to observed antibacterial properties. Studies have reported that synthetic (not naturally occurring) variants of vitamin A to chemically destabilize and/or disrupt the cell intactness of methicillin-resistant Staphylococcus aureus (MRSA) cell walls (W. Kim et al., A new class of synthetic retinoid antibiotics effective against bacterial persisters, 556 NATURE 103-107 (2018)) and other so-called “persister” bacteria (Maarten Fauvart et al., Stabbed while Sleeping: Synthetic Retinoid Antibiotics Kill Bacterial Persister Cells, (2018), https://pubag.nal.usda.gov/catalog/5981171 (last visited Jul. 9, 2020)), facilitating clearance by typical immune functions. Vitamin A has been reported as having the ability to strengthen immune responses associated with disruption of pathogen intactness. Vitamin A doses have been shown to improve autophagy (Michelle M. Coleman et al., All-trans Retinoic Acid Augments Autophagy during Intracellular Bacterial Infection, 59 AMERICAN JOURNAL OF RESPIRATORY CELL AND MOLECULAR BIOLOGY 548-556 (2018); Huang et al.; Paras K. Anand et al., Synergistic action of vitamin D and retinoic acid restricts invasion of macrophages by pathogenic mycobacteria, 41 JOURNAL OF MICROBIOLOGY, IMMUNOLOGY, AND INFECTION = WEI MIAN YU GAN RAN ZA ZHI 17-25 (2008)), particularly important against mycobacteria (e.g. tuberculosis) that have a mechanism that includes replicating inside macrophages, the cells that are otherwise the first line of defense against infection, with the role of engulfing bacteria and destroying them (the process of phagocytosis).

Other vitamin and vitamin-related compounds have been shown to have benefits in combatting infections. Vitamin E is insoluble and the properties of this vitamin have been recently reviewed (Nurul ‘Izzah Ibrahim et al., Wound Healing Properties of Selected Natural Products, 15 INTERNATIONAL JOURNAL OF ENVIRONMENTAL RESEARCH AND PUBLIC HEALTH (2018), https://www.ncbi.nlm.nih.gov/pmc/articles/PMC6266783/ (last visited Jul. 14, 2020)). The autophagy benefits of vitamin A, as described above, are reportedly complemented by co-dosing with vitamin D (Anand et al.). Synergism of oral doses combining both vitamin A and retinoic acid were shown to result in higher retinyl ester levels in lung than with doses of either compound alone (A. Catharine Ross et al., The Components of VARA, a Nutrient-Metabolite Combination of Vitamin A and Retinoic Acid, Act Efficiently Together and Separately to Increase Retinyl Esters in the Lungs of Neonatal Rats, 136 THE JOURNAL OF NUTRITION 2803-2807 (2006)).

Given the rise of the SARS-CoV-2 virus, a member of the coronavirus family, and COVID-19, the disease caused by SARS-CoV-2, it is relevant to consider known effects of vitamin A against coronaviruses. Because of known benefits of dosing, such as those described above, Vitamin A is mentioned as a compound that should be studied in a review of possible treatments for COVID-19 (Lei Zhang & Yunhui Liu, Potential interventions for novel coronavirus in China: A systematic review, 92 JOURNAL OF MEDICAL VIROLOGY 479-490 (2020)), but no such testing for vitamin A has been reported to date. Implications of a role for vitamin A in coronavirus infections or treatment (beyond SARS-CoV-2 and COVID-19) have been reported for livestock. A bovine coronavirus (BCoV) vaccine has been reported to be more effective when calves are fed vitamin-A-enriched diets and less effective upon vitamin-A-deficient diets (Jee et al.). Chickens infected with infectious bronchitis virus (IBV, a coronavirus) show reduced vitamin A status, including lower serum levels of vitamin A and transthyretin, and the situation was exacerbated when dietary vitamin A was intentionally restricted (West et al.). In these cases, control of vitamin A ‘dosing’ was only through manipulation of feedstock, thus implicating only the oral dosing route.

Accordingly, there is a long felt but unmet need for the effective provision of insoluble active ingredients to the lungs and other organs to treat or prevent infections. To that end, inhalable compositions of vitamin A suitable for direct delivery to the lungs are disclosed, which have unexpectedly been found to offer enhanced disease prophylaxis versus IM injection.

### SUMMARY

Accordingly, the present disclosure provides pharmaceutical compositions suitable for inhalation, parenteral administration, and/or oral administration that comprise an insoluble active ingredient, pharmaceutical systems comprising such pharmaceutical compositions, methods of treatment and/or prophylaxis of disease and/or disorders, and uses of such pharmaceutical compositions. In some embodiments, the insoluble active ingredient comprises an insoluble vitamin. In some embodiments the insoluble vitamin comprises a therapeutically effective dose of vitamin A, or a pharmaceutically acceptable isomer, analog, and/or derivative thereof, or a mixture thereof. In some embodiments, the insoluble vitamin comprises vitamin D, a pharmaceutically acceptable isomer, analog, and/or derivative thereof, or a mixture thereof. In some embodiments, the insoluble vitamin comprises Vitamin E, a pharmaceutically acceptable isomer, analog, and/or derivative thereof, or a mixture thereof. In some embodiments, the insoluble vitamin comprises Vitamin K, a pharmaceutically acceptable isomer, analog, and/or derivative thereof, or a mixture thereof. The formulations and systems provided by the present disclosure may be used in the treatment and/or prophylaxis of, by way of non-limiting examples, vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS).

Disclosed is a pharmaceutical composition comprising an aqueous formulation comprising an insoluble vitamin at least partially solubilized with a surfactant wherein the pharmaceutical composition is in a liquid aerosol form.

Disclosed is a pharmaceutical composition wherein the insoluble vitamin comprises vitamin A, a pharmaceutically acceptable isomer, analog, and/or derivative thereof, or a mixture thereof, vitamin D, a pharmaceutically acceptable isomer, analog, and/or derivative thereof, or a mixture thereof, vitamin E, a pharmaceutically acceptable isomer, analog, and/or derivative thereof, or a mixture thereof, or vitamin K, a pharmaceutically acceptable isomer, analog, and/or derivative thereof, or a mixture thereof.

Disclosed is a pharmaceutical composition wherein the insoluble vitamin comprises vitamin A, a pharmaceutically acceptable isomer, analog, and/or derivative thereof, or a mixture thereof.

Disclosed is a pharmaceutical composition comprising up to 6.0% (w/w) of the vitamin A, a pharmaceutically acceptable isomer, analog, and/or derivative thereof, or a mixture thereof.

Disclosed is a pharmaceutical composition wherein a weight of the surfactant is at least 3.0 times the weight of the insoluble vitamin.

Disclosed is a pharmaceutical composition wherein the remainder of the pharmaceutical composition comprises water.

Disclosed is a pharmaceutical composition wherein the pharmaceutical composition comprises a conjugate acid or a conjugate base.

Disclosed is a pharmaceutical composition wherein the insoluble vitamin comprises all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, CD437 or CD1530, or an intrinsically fluorescent retinoid analog, or a mixture thereof, a weight of surfactant which is between 3.0 times and 8.5 times the weight of the all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, CD437, or CD-1530, or an intrinsically fluorescent retinoid analog, or a mixture thereof, contained in the composition, wherein the remainder of the composition comprises water and, optionally, wherein the pH has been adjusted by addition of a pharmaceutically acceptable acid and/or pharmaceutically acceptable base.

Disclosed is a pharmaceutical composition wherein the insoluble active ingredient comprises between 0.015% (w/w) and 4.0% (w/w) of all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, CD437, or CD1530, a weight of surfactant which is between 4.0 times and 5.0 times the weight of the all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol contained in the composition, wherein the remainder of the composition comprises water and, optionally, wherein the pH has been adjusted to between 7.0 and 7.5 by addition of a pharmaceutically acceptable acid and/or pharmaceutically acceptable base.

Disclosed is a pharmaceutical composition wherein the insoluble active ingredient comprises between 0.015% (w/w) and 4.0% (w/w) of all trans retinol, or the palmitate, acetate, or maleate ester of all trans retinol, a weight of surfactant which is between 3.0 times and 8.5 times the weight of the all trans retinol, or the palmitate, acetate, or maleate ester of all trans retinol contained in the composition, wherein the remainder of the composition comprises water and, optionally, wherein the pH has been adjusted by addition of a pharmaceutically acceptable acid and/or pharmaceutically acceptable base.

Disclosed is a pharmaceutical composition wherein the insoluble active ingredient comprises between 0.015% (w/w) and 4.0% (w/w) vitamin A or a pharmaceutically acceptable isomer, analog, and/or derivative thereof, or a mixture thereof, a weight of a surfactant which is between 3.0 times and 8.5 times the weight of the vitamin A palmitate contained in the composition, wherein the remainder of the composition comprises water, and, optionally, wherein the pH has been adjusted by addition of a pharmaceutically acceptable acid and/or a pharmaceutically acceptable base.

Disclosed is a pharmaceutical composition wherein the pH of the pharmaceutical composition has been optionally adjusted to between pH 7.0 and pH 7.5.

Disclosed is a pharmaceutical composition wherein the surfactant comprises polysorbate 20, polysorbate 60, polysorbate 80, stearyl alcohol, a polyethylene glycol derivative of hydrogenated castor oil, a polyethylene glycol derivative of hydrogenated castor oil, sorbitan monolaurate, sorbitan monopalmitate, sorbitan monostearate, polyoxyethylene (20) oleyl ether, polyoxyethylene (20) cetyl ether, polyoxyethylene (10) cetyl ether, polyoxyethylene (10) oleyl ether, polyoxyethylene (100) stearyl ether, polyoxyethylene (10) stearyl ether, polyoxyethylene (20) stearyl ether, polyoxyethylene (4) lauryl ether, polyoxyethylene (20) cetyl ether, polyoxyethylene (2) cetyl ether, caprylocaproyl polyoxyl-8 glyceride, polyethylene glycol (20) stearate, polyethylene glycol (40) stearate, polyethylene glycol, polyethylene glycol (8) stearate, polyoxyl 40 stearate, poloxamer 188, polaxamer 312, or mixtures thereof.

Disclosed is a pharmaceutical composition wherein the surfactant comprises polysorbate 80.

Disclosed is a pharmaceutical composition wherein the particles formed by the insoluble active ingredient and the surfactant form particles in the configuration of micelles having a diameter of less than or equal to 500 nm.

Disclosed is a pharmaceutical composition wherein the micelles have a diameter of less than or equal to 250 nm.

Disclosed is a pharmaceutical composition according to claim 15. wherein the micelles have a diameter of less than or equal to 100 nm.

Disclosed is a pharmaceutical composition wherein the insoluble active ingredient comprises between 0.125 % and 3.0% vitamin A, or an isomer, analog, and/or derivative thereof, or a mixture thereof.

Disclosed is a pharmaceutical composition wherein the insoluble active ingredient comprises between 1.25% and 3.0% vitamin A, or an isomer, analog, and/or derivative thereof, or a mixture thereof.

Disclosed is a pharmaceutical composition wherein the surfactant comprises polysorbate 80 and the fatty acid content of the polysorbate 80 is between 58% and 100% oleic acid.

Disclosed is a pharmaceutical composition wherein the surfactant comprises polysorbate 80 and the fatty acid content of the polysorbate 80 is between 85% and 100% oleic acid.

Disclosed is a pharmaceutical composition wherein the surfactant comprises polysorbate 80 and the fatty acid content of the polysorbate 80 is greater than or equal to 98% oleic acid.

Disclosed is a pharmaceutical composition wherein the pharmaceutically acceptable acid comprises hydrochloric acid, hydrobromic acid, sulfuric acid, nitric acid, phosphoric acid, acetic acid, oleic acid, palmitic acid, stearic acid, glycolic acid, pyruvic acid, oxalic acid, maleic acid, malonic acid, succinic acid, fumaric acid, tartaric acid, citric acid, ascorbic acid, lactic acid, or tartaric acid.

Disclosed is a pharmaceutical composition wherein the pharmaceutically acceptable base comprises sodium hydroxide, ammonium hydroxide, potassium hydroxide, histidine, arginine, or lysine.

Disclosed is a pharmaceutical composition wherein the pharmaceutically acceptable acid comprises citric acid and the pharmaceutically acceptable base comprises sodium hydroxide.

Disclosed is a pharmaceutical composition further comprising one or more antibiotic compounds or mixtures thereof.

Disclosed is a pharmaceutical composition wherein the antibiotic compound or compounds comprises penicillins, tetracyclines, cephalosporins, quinolones, lincomycins, macrolides, sulfonamides, glycopeptides, aminoglycosides, carbapenems, or mixtures thereof.

Disclosed is a pharmaceutical composition further comprising one or more antibiotic or anti-viral compounds.

Disclosed is a pharmaceutical composition wherein the insoluble active ingredient comprises all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, CD437 or CD1530, or a mixture thereof, or the palmitate, acetate, or maleate ester of all trans retinol, 13-cis-retinol, 9-cis-retinol, 9,13-dicis-retinol, 3,4-didehydroretinol, CD437, CD-1530, an intrinsically fluorescent retinoid analog or the mixture thereof.

Disclosed is a pharmaceutical composition comprising a therapeutically effective amount of all trans retinol, or the palmitate, acetate, or maleate ester of all trans retinol.

Disclosed is a pharmaceutical composition comprising a therapeutically effective amount of vitamin A palmitate.

Disclosed is a pharmaceutical composition further comprising Vitamin B, Vitamin C, Vitamin D, Vitamin D, Vitamin E, Vitamin K, or an analog and/or derivative of Vitamin B, Vitamin C, Vitamin D, Vitamin E, Vitamin K, or a mixture thereof.

Disclosed is a pharmaceutical composition further comprising an anti-viral compound.

Disclosed is a pharmaceutical composition wherein the anti-viral compound comprises oseltamivir, zanamivir, peramivir, ribavirin, remdesivir, a nucleoside analog or analogs, an interferon or interferons, a protease inhibitor or inhibitors, a reverse transcriptase inhibitor, a neuramidinidase inhibitor or inhibitors, or a mixture thereof.

Disclosed is a pharmaceutical composition wherein the pharmaceutically acceptable carrier comprises a sugar or mixture of sugars.

Disclosed is a pharmaceutical composition wherein the sugar or mixture of sugars comprises glucose, arabinose, maltose, sucrose, dextrose and lactose, or a mixture thereof.

Disclosed is a pharmaceutical composition wherein the pharmaceutical composition is suitable for intravenous or intraarterial parenteral administration, oral administration, or administration by inhalation through the mouth or nose.

Disclosed is a pharmaceutical composition wherein the pharmaceutical composition is suitable for intravenous or intraarterial parenteral administration.

Disclosed is a pharmaceutical composition wherein the pharmaceutical composition is adapted for inhalation.

Disclosed is a pharmaceutical composition wherein the pharmaceutical composition does not comprise a preservative.

Disclosed is a pharmaceutical composition wherein the liquid aerosol form is disposed in a gas and the gas comprises oxygen.

Disclosed is a pharmaceutical composition wherein the liquid aerosol form is disposed in ambient air.

Disclosed is a pharmaceutical composition wherein the liquid aerosol form is disposed in a mixture of ambient air and oxygen such that the concentration of the oxygen in the mixture is above that of ambient air.

Disclosed is a pharmaceutical composition wherein the pharmaceutical composition is disposed within a sealed container.

Disclosed is a pharmaceutical composition wherein the sealed container comprises a gas.

Disclosed is a pharmaceutical composition wherein the gas comprises oxygen.

Disclosed is a pharmaceutical composition wherein the gas comprises an insoluble gas.

Disclosed is a pharmaceutical composition wherein the insoluble gas comprises nitrogen or argon.

Disclosed is a pharmaceutical composition wherein the gas comprises nitrous oxide.

Disclosed is a pharmaceutical composition further comprising vitamin D, vitamin E, vitamin K, vitamin B6, vitamin C, niacinamide, vitamin B2, vitamin B1. dexpanthenol, biotin, folic acid, vitamin B12, or a mixture thereof.

Disclosed is a pharmaceutical composition comprising an insoluble vitamin at least partially solubilized with a surfactant wherein the pharmaceutical composition is in liquid aerosol form.

Disclosed is a pharmaceutical composition wherein the insoluble vitamin comprises a therapeutically effective amount of vitamin A, or an isomer, analog, and/or derivative thereof, or a mixture thereof.

Disclosed is a pharmaceutical composition wherein the therapeutically effective amount of vitamin A, or an isomer, analog, and/or derivative thereof, or a mixture thereof, comprises all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, an intrinsically fluorescent retinoid analog or a mixture thereof.

Disclosed is a pharmaceutical composition wherein the therapeutically effective amount of vitamin A, or an isomer, analog, and/or derivative thereof, or a mixture thereof, comprises a therapeutically effective amount of all trans retinol, or the palmitate, acetate, or maleate ester of all trans retinol.

Disclosed is a pharmaceutical composition wherein the therapeutically effective amount of all trans retinol, or the palmitate, acetate, or maleate ester of all trans retinol comprises a therapeutically effective amount of vitamin A palmitate and a pharmaceutically acceptable carrier.

Disclosed is a pharmaceutical composition wherein the pharmaceutically acceptable carrier comprises a sugar or mixture of sugars.

Disclosed is a pharmaceutical composition wherein the sugar or a mixture of sugars comprises glucose, arabinose, maltose, sucrose, dextrose and lactose, or a mixture thereof.

Disclosed is a pharmaceutical composition comprising: (i) vitamin A, or a pharmaceutically acceptable isomer, analog, and/or derivative thereof, or a mixture thereof; (ii) a surfactant, and wherein the pharmaceutical composition is in aerosol form.

Disclosed is a pharmaceutical composition wherein the surfactant comprises a non-natural surfactant.

Disclosed is a pharmaceutical composition wherein the vitamin A, or a pharmaceutically acceptable isomer, analog, and/or derivative thereof, or a mixture thereof, and the surfactant form a solid particle.

Disclosed is a pharmaceutical composition wherein the solid particle has a diameter of about 3 to about 5 µm.

Disclosed is a pharmaceutical composition wherein the solid particle is sized to reach one or more portions of the respiratory zone of the lung, the conducting zone of the lung, or both portions.

Disclosed is a pharmaceutical composition wherein the solid particle has a diameter of about 0.5 to about 3 µm.

Disclosed is a pharmaceutical composition wherein the solid particle is sized to reach an alveolus.

Disclosed is a pharmaceutical composition wherein the pharmaceutical composition is a liquid and wherein the vitamin A, or a pharmaceutically acceptable isomer, analog, and/or derivative thereof, or a mixture thereof, is disposed within a micelle.

Disclosed is a pharmaceutical composition wherein the micelle has a diameter of between 1 nanometer and 1 micron.

Disclosed is a pharmaceutical composition further comprising water.

Disclosed is a pharmaceutical composition wherein the water further comprises a soluble active ingredient.

Disclosed is a pharmaceutical composition wherein the hydrophilic constituent comprises vitamin C or certain antibiotics/antivirals.

Disclosed is a pharmaceutical composition wherein the vitamin A, or a pharmaceutically acceptable isomer, analog, and/or derivative thereof, or a mixture thereof is disposed within the water.

Disclosed is a pharmaceutical composition wherein the surfactant is disposed within the water.

Disclosed is a pharmaceutical composition wherein the vitamin A, or a pharmaceutically acceptable isomer, analog, and/or derivative thereof, or a mixture thereof, is disposed within a micelle.

Disclosed is a pharmaceutical composition further comprising an insoluble constituent disposed within the micelle.

Disclosed is a pharmaceutical composition wherein the insoluble constituent comprises vitamin D, vitamin E, vitamin K, an insoluble antibiotic, an insoluble antiviral, caffeine, epinephrine, or nitrous oxide.

Disclosed is a pharmaceutical composition further comprising an additional micelle wherein the micelle and the additional micelle have an average diameter of between one micron and one nanometer.

Disclosed is a pharmaceutical composition wherein the remainder of the pharmaceutical composition comprises water.

Disclosed is a pharmaceutical composition wherein the pharmaceutical composition comprises a conjugate base of a pharmaceutically acceptable acid.

Disclosed is a pharmaceutical composition wherein the pharmaceutical composition comprises a conjugate acid of a pharmaceutically acceptable base.

Disclosed is a pharmaceutical composition suitable for inhalation consisting essentially of an insoluble vitamin at least partially solubilized with a surfactant wherein the pharmaceutical composition is in liquid aerosol form.

Disclosed is a system for use in the prophylaxis or treatment of a vitamin deficiency disorder, hyperoxia damage, oxidative lung injury (lung damage), lung damage caused by chemical or environmental exposure, lung damage caused by a biological/infectious agent, lung damage caused by a chemical, biological, or radio-nuclear (CBRN) agent, and lung damage caused by radio-nuclear exposure, wherein the system comprises a therapeutically effective dose of a disclosed pharmaceutical composition and a device suitable for generating the liquid aerosol form thereby forming a plurality of liquid droplets wherein the liquid droplets produced by the system have a mass median aerodynamic diameter of less than 15 microns.

Disclosed is a system wherein the liquid droplets have a mass median aerodynamic diameter of less than 10 microns.

Disclosed is a system wherein the liquid droplets have a mass median aerodynamic diameter of less than 5 microns.

Disclosed is a system wherein the liquid droplets have a mass median aerodynamic diameter of less than 3 microns.

Disclosed is a system for use in the prophylaxis or treatment of a vitamin deficiency disorder, hyperoxia damage, oxidative lung injury (lung damage), lung damage caused by chemical or environmental exposure, lung damage caused by a biological/infectious agent, lung damage caused by a chemical, biological, or radio-nuclear (CBRN) agent, and lung damage caused by radio-nuclear exposure, wherein the system comprises a therapeutically effective dose of a disclosed pharmaceutical composition and a device suitable for generating the solid aerosol form thereby forming a plurality of solid particles wherein the solid particles produced by the system have a mass median aerodynamic diameter of less than 15 microns.

Disclosed is a system wherein the solid particles have a mass median aerodynamic diameter of less than 10 microns.

Disclosed is a system wherein the solid particles have a mass median aerodynamic diameter of less than 5 microns.

Disclosed is a system wherein the solid particles have a mass median aerodynamic diameter of less than 3 microns.

Disclosed is a pharmaceutical composition for use in the treatment or prophylaxis in a patient in need thereof of a vitamin deficiency disorder, hyperoxia damage, oxidative lung injury (lung damage), lung damage caused by chemical or environmental exposure, lung damage caused by a biological/infectious agent, lung damage caused by a chemical, biological, or radio-nuclear (CBRN) agent, and lung damage caused by radio-nuclear exposure.

Disclosed is a pharmaceutical composition wherein the vitamin deficiency disorder comprises vitamin A deficiency disorder.

Disclosed is a pharmaceutical composition wherein the vitamin A deficiency disorder comprises bronchopulmonary dysplasia, retinopathy of prematurity, neonatal sepsis, hypovitaminosis A, hospital-acquired sepsis, sepsis from premature rupture of membranes, meningitis, pneumonia, necrotizing enterocolitis, radiation-induced pneumonitis, a viral infection. a bacterial infection, or a combination of such disorders.

Disclosed is a pharmaceutical composition wherein the viral infection comprises a Sendai virus infection, a SARS-CoV-2 infection, a coronavirus infection, a bovine coronavirus infection, an infectious bronchitis virus infection, an influenza virus infection, or a measles virus infection.

Disclosed is a pharmaceutical composition wherein the bacterial infection comprises a Mycoplasma tuberculosis infection, a Mycoplasma pneumoniae infection, a Citrobacter rodentium infection, a Streptococcus pneumoniae infection, or a methicillin-resistant Staphylococcus aureus infection.

Disclosed is a pharmaceutical composition wherein the patient is a human born prematurely or a neonate.

Disclosed is a pharmaceutical composition for use of achieving healthy peroxisome proliferator-activated receptor gamma signaling in lung tissue.

Disclosed is a pharmaceutical composition for use of achieving healthy normal levels of activin receptor-like kinase 5 in lung tissue.

Disclosed is a pharmaceutical composition for use of achieving healthy normal levels of surfactant protein C.

Disclosed is a pharmaceutical composition for use in preventing β-catenin reaching concentration levels consistent with the initiation or continuation of biological processes leading to lung physiology malformation.

Disclosed is a pharmaceutical composition for use of achieving healthy normal levels of lung tissue physiology.

Disclosed is a pharmaceutical composition for use of achieving healthy normal alveolar morphology.

Disclosed is a pharmaceutical composition for use of achieving healthy normal alveolar count.

Disclosed is a pharmaceutical composition for use of achieving healthy normal alveolar size.

Disclosed is a pharmaceutical composition for use of achieving healthy normal alveolar septal thickness.

Disclosed is a pharmaceutical composition for use in achieving or maintaining healthy normal lung maturation.

Disclosed is a pharmaceutical composition for use in administration by inhalation for avoiding damage to normal healthy lung physiology or initiation of biological shock pathways leading to malformation of lung in any manner differing from healthy normal lung physiology that may be initiated by biological or chemical damage to lung tissue.

Disclosed is a pharmaceutical composition wherein the healthy normal lung maturation status comprises one or more favorable characteristics of lung tissue histology, one or more favorable levels of one or more indicators of metabolic status, or a combination thereof.

Disclosed is a pharmaceutical composition wherein the lung histology characteristic comprises maintenance of healthy levels of alveolar count, alveolar size, alveolar septal thickness, alveolar neutrophil infiltration, interstitial neutrophil infiltration, or a combination thereof.

Disclosed is a pharmaceutical composition wherein the metabolic status comprises maintenance of healthy levels of Surfactant Protein A, Surfactant Protein B, Surfactant Protein C, Surfactant Protein D, Peroxisome Proliferator-activated Receptor Gamma, BCL-2 Protein, BCL-2 Associated X Protein, Retinoic Acid X Receptor Alpha, Retinoic Acid X Receptor Beta, Retinoic Acid X Receptor Gamma, Vascular Endothelial Growth Factor, T-complex Protein 1 Subunit Alpha (also known as CTP:Phosphocholine Cytidylyltransferase Subunit Alpha), Fetal Liver Kinase 1, Beta-catenin, Activin Receptor-Like Kinase 5, or lung surfactant phospholipid synthesis rate, either singly or in a combination thereof.

Disclosed is a pharmaceutical composition wherein the biological or chemical damage is caused by exposure to excess oxygen, any chemical that damages lung tissue, viral lung infection, bacterial lung infection, a genetic defect or a disease, either singly or a combination thereof.

Disclosed is a pharmaceutical composition wherein the chemical damage is a hyperoxic condition.

Disclosed is a pharmaceutical composition wherein the biological shock pathways include the wnt pathway, apoptotic responses, necrosis, initiation of fibrosis, or initiation of scarring, either singly or a combination thereof.

Disclosed is a pharmaceutical composition for use in preventing a protein achieving concentration levels consistent with the initiation or continuation of biological processes leading to lung physiology malformation wherein the protein comprises Beta-catenin, Activin Receptor-like Kinase 5, Lymphoid Enhancing Binding Factor 1, Transforming Growth Factor Beta, calponin, fibronectin, or one or more proteins of the wnt pathway, either alone or in combination.

Disclosed is a pharmaceutical composition for treating a human or an animal.

Disclosed is a pharmaceutical composition for use for treating a prematurely born neonate.

Disclosed is a pharmaceutical composition for use in the treatment or prophylaxis of an infection caused by one or more infectious agents.

Disclosed is a pharmaceutical composition for use in the treatment or prophylaxis of an infection caused by one or more bacterial agents, one or more viral agents, one or more mycobacterial agents, or a combination thereof.

Disclosed is a pharmaceutical composition for use in the treatment or prophylaxis of an infection caused by one or more antibiotic resistant bacteria.

Disclosed is a pharmaceutical composition for use in the treatment or prophylaxis of an infection caused by a methicillin resistant Staphyloccus aureus.

Disclosed is a pharmaceutical composition for use in the treatment or prophylaxis of an infection caused by a coronavirus.

Disclosed is a pharmaceutical composition for use in the treatment or prophylaxis of an infection caused by SARS-CoV-2.

Disclosed is a pharmaceutical composition for use in the treatment or prophylaxis of an infection or infections that target the respiratory system as the primary location of entry.

Disclosed is a pharmaceutical composition for use in the treatment or prophylaxis of an infection or infections that target the lung, the nasal pathway, the oral cavity, or a combination thereof.

Disclosed is a pharmaceutical composition for use in minimizing lung damage caused by an infectious agent or agents.

Disclosed is a pharmaceutical composition for use in minimizing the onset of acute respiratory distress syndrome.

Disclosed is a pharmaceutical composition for use in the treatment of prophylaxis of a condition or conditions facilitated by the presence of an infectious agent or agents.

Disclosed is a pharmaceutical composition for use in the treatment or prophylaxis of iatrogenic damage to lung tissue caused by the introduction of increased oxygen levels used to counter reduced pulmonary function caused by an infectious agent or agents.

Disclosed is a pharmaceutical composition for use of achieving healthy normal alveolar morphology in a patient that has been treated for an infectious agent or agents.

Disclosed is a pharmaceutical composition for use in achieving normal alveolar septal thickness, radial alveolar count, or alveolar mean linear intercept, alone or in combination.

Disclosed is a pharmaceutical composition for use in the treatment of prophylaxis of pneumonia, pneumonitis, other infections, asthma, angina, exacerbated arthritis, allergies, and intercalated infections, or a combination thereof.

Disclosed is a pharmaceutical composition for use in the suppression of over-stimulation of the immune response in a patient in need thereof.

Disclosed is a pharmaceutical composition for use in the treatment of infection-induced vitamin A deficiency.

Disclosed is a pharmaceutical composition for use in supporting a patient’s immune response.

Disclosed is a pharmaceutical composition for use in the stimulation of phagosome maturation or recruiting of white blood cells to the site of an infection.

Disclosed is a pharmaceutical composition for use in improving the efficacy of a vaccine against an infectious agent in a patient.

Disclosed is a pharmaceutical composition for use in countering suppressed levels of bloodborne vitamin A, suppressed levels of lung tissue vitamin A, or a combination thereof induced by systemic response to an infectious agent.

Disclosed is a pharmaceutical composition wherein the vitamin A deficiency disorder is bronchopulmonary dysplasia or retinopathy of prematurity.

Disclosed is a pharmaceutical composition wherein the vitamin A deficiency disorder is bronchopulmonary dysplasia.

Disclosed is a pharmaceutical composition wherein the pharmaceutical composition further comprises an anti-viral compound comprising oseltamivir, zanamivir, peramivir, ribavirin, remdesivir, a nucleoside analog or analogs, an interferon or interferons, a protease inhibitor or inhibitors, a reverse transcriptase inhibitor or inhibitors, and neuramidinidase inhibitor or inhibitors, or a mixture thereof.

Disclosed is a method of achieving or maintaining healthy normal lung maturation status comprising administering by inhalation a disclosed pharmaceutical composition.

Disclosed is a method wherein the healthy normal lung maturation status comprises one or more favorable characteristics of lung tissue histology, one or more favorable levels of one or more indicators of metabolic status, or a combination thereof.

Disclosed is a method wherein the lung histology characteristic comprises maintenance of healthy levels of alveolar count, healthy levels of alveolar size, healthy levels of alveolar septal thickness, or a combination thereof.

Disclosed is a method wherein the metabolic status comprises maintenance of healthy levels of Surfactant Protein A, Surfactant Protein B, Surfactant Protein C, Surfactant Protein D, Peroxisome Proliferator-activated Receptor Gamma signaling, BCL-2 Protein, BCL-2 Associated X Protein, Retinoic Acid X Receptor Alpha, Retinoic Acid X Receptor Beta, Retinoic Acid X Receptor Gamma, Vascular Endothelial Growth Factor, T-complex Protein 1 Subunit Alpha (also known as CTP:Phosphocholine Cytidylyltransferase Subunit Alpha), Fetal Liver Kinase 1, or lung surfactant phospholipid synthesis rate, either singly or in a combination thereof.

Disclosed is a method of avoiding damage to normal healthy lung physiology or initiation of biological repair or shock pathways leading to malformation of lung in any manner differing from healthy normal lung physiology that may be initiated by biological or chemical damage to lung tissue, a vitamin A deficiency, hyperoxia damage, oxidative lung injury (lung damage), lung damage caused by chemical or environmental exposure, lung damage caused by bacterial infections and/or viral infections, lung damage caused by a chemical, biological, or radio-nuclear (CBRN) agent, and lung damage caused by radio-nuclear exposure, comprising administering by inhalation a disclosed pharmaceutical composition.

Disclosed is a method wherein the biological or chemical damage is caused by exposure to excess oxygen, any chemical that damages lung tissue, viral lung infection, bacterial lung infection, a genetic defect, or a disease, either singly or a combination thereof.

Disclosed is a method wherein the chemical damage is a hyperoxic condition.

Disclosed is a method wherein the biological repair or shock pathways comprise the wnt pathway, apoptotic responses, necrosis, initiation of fibrosis, or initiation of scarring, either singly or a combination thereof.

Disclosed is a method wherein proteins comprising Beta-catenin, Activin Receptor-like Kinase 5, Lymphoid Enhancing Binding Factor 1, Transforming Growth Factor Beta, calponin, fibronectin, or one or more proteins of the wnt pathway, either alone or in combination, are prevented from achieving concentration levels consistent with the initiation or continuation of biological processes leading to lung physiology malformation.

Disclosed is a method wherein which the treatment is applied to a human or an animal.

Disclosed is a method wherein the treatment is applied to a prematurely born neonate.

Disclosed is a method of achieving healthy peroxisome proliferator-activated receptor gamma signaling in lung tissue, comprising administering by inhalation a disclosed pharmaceutical composition.

Disclosed is a method of achieving healthy normal levels of activin receptor-like kinase 5 in lung tissue, comprising administering by inhalation a disclosed pharmaceutical composition.

Disclosed is a method of achieving healthy normal levels of surfactant protein C, comprising administering by inhalation a disclosed pharmaceutical composition.

Disclosed is a method of preventing β-catenin reaching concentration levels consistent with the initiation or continuation of biological processes leading to lung physiology malformation, comprising administering by inhalation a disclosed pharmaceutical composition.

Disclosed is a method of achieving healthy normal development of lung tissue physiology, comprising administering by inhalation a disclosed pharmaceutical composition.

Disclosed is a method of achieving healthy normal alveolar morphology, comprising administering by inhalation a disclosed pharmaceutical composition.

Disclosed is a method of achieving healthy normal alveolar count, comprising administering by inhalation a disclosed pharmaceutical composition.

Disclosed is a method of achieving healthy normal alveolar size, comprising administering by inhalation a disclosed pharmaceutical composition.

Disclosed is a method of achieving healthy normal alveolar septal thickness, comprising administering by inhalation a disclosed pharmaceutical composition.

Disclosed is a method for the treatment or prophylaxis of an infection caused by one or more infectious agents comprising administration to a patient in need thereof of a therapeutically effective amount of a disclosed pharmaceutical composition.

Disclosed is a method wherein the administration is by inhalation.

Disclosed is a method wherein the administration is by intravenous or intraarterial parenteral administration,

Disclosed is a method wherein the administration is by oral administration.

Disclosed is a method wherein the one or more infectious agents is one or more bacterial agents, one or more viral agents, one or more mycobacterial agents, or a combination thereof.

Disclosed is a method wherein the one or more infectious agents is one or more antibiotic resistant bacteria.

Disclosed is a method wherein the is the one or more antibiotic resistant bacteria comprises methicillin resistant Staphylococcus aureus.

Disclosed is a method wherein the one or more viral agents comprises a coronavirus.

Disclosed is a method wherein the coronavirus is SARS-CoV-2.

Disclosed is a method wherein the infectious agent or agents target the respiratory pathway as the primary location of entry.

Disclosed is a method wherein the target of the respiratory pathway is the lung, the nasal passages, the oral cavity, or a combination thereof.

Disclosed is a method for minimizing lung damage caused by the infectious agent or agents.

Disclosed is a method wherein the minimizing lung damage minimizes the onset of acute respiratory distress syndrome.

Disclosed is a method for the treatment or prophylaxis of a condition or conditions facilitated by the presence of the infectious agent or agents.

Disclosed is a method wherein the condition being treated or prevented is iatrogenic damage to lung tissue caused by the introduction of increased oxygen levels used to counter reduced pulmonary function caused by an infectious agent or agents.

Disclosed is a method wherein the treatment or prophylaxis achieves healthy normal alveolar morphology in a patient that has been treated for an infectious agent or agents.

Disclosed is a method wherein the treatment or prophylaxis achieves healthy normal alveolar septal thickness, radial alveolar count, or alveolar septal thickness, alone or in combination.

Disclosed is a method wherein the condition is selected from pneumonia, pneumonitis, an infection, asthma, angina, exacerbated arthritis, allergies, radiation-induced pneumonitis, sepsis, intercalated infections, or a combination thereof.

Disclosed is a method of suppressing the over-stimulation of the immune response in a patient in need thereof, comprising administering a therapeutically effective amount of a disclosed pharmaceutical composition.

Disclosed is a method of treating infection-induced vitamin A deficiency in a patient in need thereof, comprising administering a therapeutic effective amount of a disclosed pharmaceutical composition.

Disclosed is a method of supporting a patient’s immune response, comprising administering a disclosed pharmaceutical composition.

Disclosed is a method wherein the supporting comprises stimulation of phagosome maturation or recruiting of white blood cells to the site of an infection.

Disclosed is a method of improving the efficacy in a patient of a vaccine against an infectious agent, comprising administering a disclosed pharmaceutical composition.

Disclosed is a method of countering suppressed levels of bloodborne vitamin A, suppressed levels of lung tissue vitamin A, or a combination thereof induced by systemic response to an infectious agent in a patient, comprising administering by inhalation a disclosed pharmaceutical composition.

Disclosed is a method of prophylactically protecting against hyperoxia damage to a lung, comprising administering to a subject the disclosed pharmaceutical composition.

Disclosed is a method of preventing iatrogenic damage to a lung, comprising administering to a subject the disclosed pharmaceutical composition.

Disclosed is a method of treating radiation exposure of a lung, comprising administering to a subject a disclosed pharmaceutical composition.

Disclosed is a method of treating oxidative damage in a lung, comprising administering to a subject a disclosed pharmaceutical composition.

Disclosed is a method of treating inhalation of radioactive particles into a lung, comprising administering to a subject a disclosed pharmaceutical composition.

Disclosed is a method of treating a lung that has been exposed to a noxious chemical, comprising administering to a subject a disclosed pharmaceutical composition.

Disclosed is a method wherein the noxious chemical comprises chlorine, phosphine, or smoke from a fire.

Disclosed is a method of treating vitamin deficiency disorders, oxidative lung injury (lung damage), lung damage caused by chemical or environmental exposure, lung damage caused by a biological/infectious agent, lung damage caused by a chemical, biological, or radio-nuclear (CBRN) agent, or lung damage caused by radio-nuclear exposure, comprising administering to a subject in need thereof a therapeutically effective amount of a disclosed composition.

Disclosed is a method wherein the biological/infectious agent causes a bacterial infection.

Disclosed is a method wherein the biological/infectious agent causes a viral infection.

Disclosed is a method wherein the lung damage caused by radio-nuclear exposure comprises acute radiation-induced lung injury/damage

Disclosed is a method wherein the lung damage caused by radio-nuclear exposure comprises chronic lung injury/damage.

Disclosed is a method wherein the lung damage caused by radio-nuclear exposure comprises delayed effects of acute radiations exposure (DEARS).

Disclosed is a method wherein the vitamin deficiency disorder comprises nutritional insufficiency, premature birth, genetic defect, or a disorder induced pursuant to infection or disease states.

### BRIEF DESCRIPTION OF THE FIGURES

FIG. 1A discloses histomorphology images of neonatal lung tissue, hyperoxia damage and effects of vitamin A dosing by inhalation or injection. In this and all subsequent figures (unless otherwise noted), the extent of statistical differentiation (by ANOVA) between groups, pairwise, according to the following legend: p<0.05 (*), p<0.01 (**), p<0.001 (***), or p<0.0001 (****).

FIG. 1B discloses tabular data used to prepare the graphs set forth in FIG. 1A. Physiological metrics of alveoli, derived from histopathology imaging of lung tissue, of healthy control animals (left-most column) are compared to animals exposed to damaging hyperoxia without or with vitamin A treatment. Numbers in brackets represent pairwise statistical comparison (ANOVA) in which p was below at most 0.05, with symbol indicating comparison group as follows: * to healthy normal, # to untreated hyperoxia, ^ to hyperoxia treated with IM vitamin A. In no metric did IM vitamin A dosing result in a statistical difference versus untreated hyperoxia. Each value represents average ± standard deviation of results from 6 animals.

FIG. 2 discloses histomorphology comparison of lung tissue, showing the result of oral versus inhaled vitamin A dosing upon hyperoxic lung damage.

FIG. 3A discloses levels of proteins associated with lung maturation determined by western blot.

FIG. 3B discloses tabular data used to prepare the graphs set forth in FIG. 3A. Symbols in table represent pairwise comparison: ^ versus health normal; # versus untreated hyperoxia. Statistical p value as indicated in graph (symbols as in FIG. 1A). Each value represents average ± standard deviation of results from 6 animals.

FIG. 4A discloses levels of proteins associated with damage repair or cellular shock pathways determined by western blot.

FIG. 4B discloses the tabular data used to prepare the graphs set forth in FIG. 4A. Symbols in table represent pairwise comparison: ^ versus health normal; # versus untreated hyperoxia. Statistical p value as indicated in graph (symbols as in FIG. 1A).

FIG. 5 discloses two-color fluorescence immunostaining of intracellular proteins in fixed lung tissue sections. Staining intensity of PPARγ and β-catenin confirm the trends shown in the data in FIGS. 3A and 4A.

FIG. 6A. Changes in gene expression levels upon hyperoxia, and the effects of inhaled vitamin A dosing. Gene expression levels are first normalized to tubulin level (a ubiquitously expressed gene) then reported as fold change from gene expression levels of healthy controls (21% O₂).

FIG. 6B discloses the tabular data used to prepare the graphs set forth in FIG. 6A.

FIG. 7A discloses immunostaining and count of neutrophil infiltration.

FIG. 7B discloses the tabular data used to prepare the graphs set forth in FIG. 7A. Statistical analyses: p<0.001 for each of the 3 possible group pairwise comparisons shown.

FIGS. 8A1, 8A2, 8B, 8C1, and 8C2 disclose the effects of injected or inhaled vitamin A under normal oxygen conditions.

FIG. 8A1 discloses Western blots and quantitation of proteins involved in lung maturation.

FIG. 8A2 discloses the tabular data used to prepare the graphs set forth in FIG. 8A1. * p<0.01, # p<0.001; ^p<0.0001. Each value represents average ± standard deviation of results from 6 animals.

FIG. 8B discloses immunofluorescence microscopy and quantitation of proteins involved in lung maturation. The fold-change vs health controls for proteins depicted in the bar graphs: PPAR 1.35 ± 0.08 ^, SP-B 1.35 ± 0.06 *, VEGF 1.23 ± 0.09 *, RXR-α 1.33 ± 0.11 #, RXR-β 0.79 ± 0.06 #, RXR-γ 1.01 ± 0.03. * p<0.05; # p<0.01; ^ p<0.001. Each value represents average ± standard deviation of results from 6 animals.

FIG. 8C1 discloses radiolabeled choline incorporation.

FIG. 8C2 discloses the tabular data used to prepare the graphs set forth in FIG. 8C1. * indicates p<0.05 versus healthy controls.

FIG. 9A discloses serum vitamin A levels. Newborn Sprague-Dawley rat pups (n=8 each group) were dosed with vitamin A (or vehicle) by injection (IM) or inhalation (Nebulizer) on postnatal days 1, 3, 5, and 7, and serum was analyzed 4-6 hours after the day 7 dose.

FIG. 9B discloses the tabular data used to prepare the graphs set forth in FIG. 9A.

### DETAILED DESCRIPTION

### 1) Pharmaceutical Compositions

In some embodiments, the pharmaceutical composition comprises an insoluble active ingredient and a surfactant wherein the pharmaceutical composition is in aerosol form. In some embodiments, the insoluble active ingredient comprises an insoluble vitamin.

In some embodiments, the insoluble vitamin comprises vitamin A or a pharmaceutically acceptable isomer, analog, and/or derivative thereof, or a mixture thereof. In some embodiments, the insoluble active ingredient comprises an insoluble antibiotic or an insoluble antiviral. In some embodiments, the insoluble active ingredient is present in a micelle. In some embodiments, the insoluble active ingredient is present at 500, 200, 100, 95, 90, 85, 80, 75, 70, 65, 60, 55, 50, 45, 40, 35, 30, 25, 20, 15, 10, 5, and 1 gram per liter; 500, 200, 100, 95, 90, 85, 80, 75, 70, 65, 60, 55, 50, 45, 40, 35, 30, 25, 20, 15, 10, 5, 1 milligram per liter; and 500, 200, 100, 95, 90, 85, 80, 75, 70, 65, 60, 55, 50, 45, 40, 35, 30, 25, 20, 15, 10, 5, 1, 0.9, 0.8, 0.7, 0.6, 0.5, 0.4, 0.3, 0.2, 0.1, 0.09, 0.08, 0.07, 0.06, 0.05, 0.04, 0.03, 0.02, and 0.01 micrograms per liter of the pharmaceutical composition in aerosol form. Ranges with lower and upper values having any of the disclosed concentrations of insoluble active ingredient are disclosed with the proviso that the upper value is greater than the lower value. In some embodiments, the lower limit of the weight of the surfactant is defined by the smallest amount that is capable of solubilizing the amount of the insoluble active ingredient or ingredients. In some embodiments, the upper limit of the weight of the surfactant is defined by the pharmaceutically allowed maximum, defined, for example, by the limit of toxicity known for the surfactant. In some embodiments, the weight of the surfactant is 50, 25, 10, 9, 8, 7, 6, 5.5, 5.4, 5.3, 5.2, 5.1, 5, 4.9, 4.8, 4.7, 4.6, 4.5, 4.4, 4.3, 4.2, 4.1, 4.0, 3.9, 3.8 3.7, 3.6, 3.5, 3.4, 3.3, 3.2, 3.1, 3, 2, and 1 times the weight of the insoluble active ingredient. In some embodiments, the weight of the surfactant is at least 3.0 times the weight of the insoluble active ingredient. Ranges with lower and upper values having any of the disclosed weights of surfactant are disclosed with the proviso that the upper value is greater than the lower value. In some embodiments, the weight of surfactant is between 4.0 times and 5.0 times the weight of the insoluble active ingredient.

Vitamin A, or retinol has the following structure:

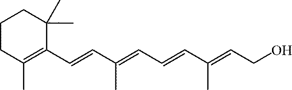 [SMILES: CC1=C(/C=C/C(C)=C/C=C/C(C)=C/CO)C(C)(C)CCC1]

It should be noted that this structure depicts the “all trans” form of retinol, which is a common form of vitamin A used therapeutically. A variety of other isomers or analogs exist that contain one or more cis bonds or other alterations to the all trans bond configuration, including, for example, 13-cis-retinol (also known as isotretinoin), 9-cis-retinol, 9,13-dicis-retinol and 3,4-didehydroretinol.

Other analogs of vitamin A are also known, and of particular interest are those with demonstrated biological activity. For example, recently, a family of intrinsically fluorescent analogs of vitamin A and other retinoids have been synthesized and many in this family are shown to be biologically active at levels similar to vitamin A palmitate (as described in, for example, Chisholm et al. “Fluorescent Retinoic Acid Analogues as Probes for Biochemical and Intracellular Characterization of Retinoid Signaling Pathways”, ACS Chem. Biol. 2019, 14, 3, 369-377). The fluorescent nature of these analogs may be advantageous in determining the location, amount, and clearance of the analogs in vivo.

It should be further noted that other common forms of vitamin A are esterified, often with palmitate, acetate, maleate, or with other saturated fatty acids. Any pharmaceutically acceptable isomer, analog, and/or and/or derivative thereof may be modified in this manner, including, by way of non-limiting examples, to form all-trans retinyl palmitate, all-trans retinyl acetate, 9-cis-retinyl palmitate, 9-cis-retinyl acetate, isotretinoin palmitate, and other similar molecules.

There are various units of measure that can describe vitamin A content or concentration in embodiments of the present disclosure. The issue is rendered somewhat complicated because some units of measure, such as mass of a particular vitamin A molecule per unit volume, does not convey the same amount of vitamin A as, for example, an IU dose, since IU is normalized to the amount of biologically active retinol content. Individual molecules described generally as vitamin A isomers, analogs, or variants each have a unique molecular weight. Using three different vitamin A molecules that are among some embodiments of the present disclosure as non-limiting examples, the molecular weight is 528.85 grams/mole (g/mol) for vitamin A palmitate, 328.5 g/mol for vitamin A acetate, and 286.5 g/mol for retinol. The use of moles, rather than weight or mass, serves to normalize the description of concentration to the number of molecules in a unit volume, a nomenclature that facilitates comparison of concentration of different molecules (and their different molecular weights). Continuing this non-limiting example with the same three example molecules, separate formulations bearing 528.85 grams per liter (g/L) of vitamin A palmitate, 328.5 g/L of vitamin A acetate, or 286.5 g/L of retinol all have the same concentration of 1 mole/L (or simply ‘molar’ or M), normalized by this molar nomenclature despite the different masses required for each individual vitamin A variant. Similarly, for vitamin A, International Unit (IU) nomenclature is used to normalize the amount or concentration of vitamin A in a formulation, and helps standardize the nomenclature for, for example, a dose of a pharmaceutical formulation. By way of a non-limiting example, the following solutions each have 50,000 IU/mL vitamin A, despite having different masses of the constituent vitamin A: 2.75% (w/w - or 2.75 g per 100 mg of total final solution mass) vitamin A palmitate, 1.72% w/w vitamin A acetate, or 1.50% w/w retinol. Or, in related nomenclature of milligrams per milliliter (mg/mL), it would be understood that 27.5 mg/mL vitamin A palmitate, 17.2 mg/mL vitamin A acetate, or 15.0 mg/mL retinol each refer to the same solution of 50,000 IU/mL vitamin A. The mass of any vitamin A molecule that is required to achieve a desired concentration as stated in IU per volume or moles per volume similarly depends on the molecular weight of the specific vitamin A molecule being used. Similarly, comparison between formulations comprising different vitamin A molecules is more accurately performed by evaluating standardized units of measure such as IU/mL or molar concentration, rather than mass or weight per mL.

The desired pharmaceutical outcomes may result from dosing of individual forms of retinol or mixtures of them. Vitamin A esters and various cis/trans isomers (as above) are possible constituents. Esters of retinol include the palmitate, acetate, maleate or similar compounds. Dosing may be referred to in terms of International Units, describing the amount of retinol delivered regardless of the molecular form used in the pharmaceutical formulation. Other nomenclatures are sometimes used, including the USP unit, equal to IU, or the Retinal Activity Equivalent (RAE), for which 1 IU is equal to 0.3 RAE. Dosing may also be referred by mass, such as grams or milligrams, or by molar content, such as moles or millimoles. In some embodiments, the dose of vitamin A, or a pharmaceutically acceptable isomer, analog, and/or derivative thereof, or amixture thereof, delivered to the subject is 100,000, 90,000, 80,000, 70,000, 60,000,50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 IU per dose. Ranges with lower and upper values having any of the disclosed doses of vitamin A, or a pharmaceutically acceptable isomer, analog, and/or derivative thereof, or a mixture thereof, are disclosed with the proviso that the upper value is greater than the lower value. In some embodiments, the dose of vitamin A, or a pharmaceutically acceptable isomer, analog, and/or derivative thereof, or a mixture thereof, may be determined based on body mass of the human or animal being dosed. In such embodiments, the dose of vitamin A, or a pharmaceutically acceptable isomer, analog, and/or derivative thereof, or a mixture thereof, is 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 IU/kg per dose. Ranges with lower and upper values having any of the disclosed doses of vitamin A, or a pharmaceutically acceptable isomer, analog, and/or derivative thereof, or a mixture thereof, are disclosed with the proviso that the upper value is greater than the lower value.

Surfactants for use according to the present disclosure include, but are not limited to, polysorbate 20 (for example Tween® 20, polysorbate 60 (for example Tween® 60), polysorbate 80 (for example Tween® 80), stearyl alcohol, a polyethylene glycol derivative of hydrogenated castor oil (for example Cremophor® RH 40), a polyethylene glycol derivative of hydrogenated castor oil for example Cremophor® RH 60), sorbitan monolaurate (for example Span® 20), sorbitan monopalmitate (for example Span® 40), sorbitan monostearate (for example Span® 60), polyoxyethylene (20) oleyl ether (for example Brij® 020), polyoxyethylene (20) cetyl ether (for example Brij® 58), polyoxyethylene (10) cetyl ether (for example Brij® C10), polyoxyethylene (10) oleyl ether (for example Brij® O10), polyoxyethylene (100) stearyl ether (for example Brij® S100), polyoxyethylene (10) stearyl ether (for example Brij® S10), polyoxyethylene (20) stearyl ether (for example Brij® S20), polyoxyethylene (4) lauryl ether (for example Brij® L4), polyoxyethylene (20) cetyl ether (for example Brij® 93), polyoxyethylene (2) cetyl ether (for example Brij® S2), caprylocaproyl polyoxyl-8 glyceride (for example Labrasol®), polyethylene glycol (20) stearate (for example Myrj™ 49), polyethylene glycol (40) stearate (for example Myrj™ S40), polyethylene glycol (100) stearate (for example Myrj™ S100), polyethylene glycol (8) stearate (for example Myrj™ S8), and polyoxyl 40 stearate (for example Myrj™ 52), triblock copolymers of polyoxypropylene and flanked by polyoxyethylene (so-called poloxamers, including Poloxamer 188, Poloxamer 331 and related variants), and mixtures thereof.

In an embodiment of the present disclosure, the surfactant is polysorbate 80.

Polysorbate 80 (polyoxyethylene (20) sorbitan monooleate), has the following general structure:

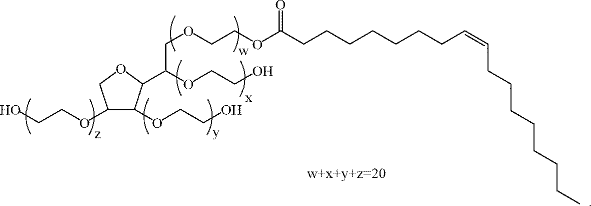

With regard to the fatty acid content of polysorbate 80, the United States Pharmacopeia (“Polysorbate 80” monograph, 2016 The United States Pharmacopeial Convention, available at https://www.usp.org/sites/default/files/usp/document/harmonization/excipients/polysorba te_80.pdf), and other national formularies, have formalized acceptance criteria for the fatty acid oleic acid content as not less than 58%. Acceptance criteria also includes myristic acid at no more than (NMT) 5.0%, palmitic acid NMT 16.0%, palmitoleic acid NMT 8.0%, stearic acid NMT 6.0%, linoleic acid NMT 18.0%, and linolenic acid NMT to 4.0%. In some embodiments, the fatty acid oleic acid content is 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 %. In some embodiments, the myristic acid is 5.0, 4.5, 4.0, 3.5, 3.0, 2.5, 2.0, 1.5, 1.0, 0.9, 0.8, 0.7, 0.6. 0.5, 0.4, 0.2, or 0.1 %. In some embodiments, the palmitic acid content is 16.0, 15.0, 14.0, 13.0, 12.0, 11.0, 10.0, 9.0, 8.0, 7.0, 6.0, 5.0, 4.5, 4.0, 3.5, 3.0, 2.5, 2.0, 1.5, 1.0, 0.9, 0.8, 0.7, 0.6. 0.5, 0.4, 0.2, or 0.1 %. In some embodiments, the stearic acid content is 6.0, 5.0, 4.5, 4.0, 3.5, 3.0, 2.5, 2.0, 1.5, 1.0, 0.9, 0.8, 0.7, 0.6. 0.5, 0.4, 0.2, or 0.1 %. In some embodiments, the linoleic acid content is 18.0, 17.0, 16.0, 15.0, 14.0, 13.0, 12.0, 11.0, 10.0, 9.0, 8.0, 7.0, 6.0, 5.0, 4.5, 4.0, 3.5, 3.0, 2.5, 2.0, 1.5, 1.0, 0.9, 0.8, 0.7, 0.6. 0.5, 0.4, 0.2, or 0.1 %. In some embodiments, the linolenic acid content is 4.0, 3.5, 3.0, 2.5, 2.0, 1.5, 1.0, 0.9, 0.8, 0.7, 0.6. 0.5, 0.4, 0.2, or 0.1 %.

Polysorbate 80 at the USP acceptable level of purity may be used in the compositions of the present disclosure as may be formulations of polysorbate 80 at higher levels of purity, for example between 85% and 100% oleic acid (for example, Super-Refined™ Polysorbate available from Croda), and greater than 98% oleic acid (for example, Polysorbate 80 (HX2)™ available from NOF).

In another embodiment of the present disclosure, the formulation can be a dry powder comprising the insoluble active ingredient mixed with a sufficient amount of a carrier. In some embodiments, the insoluble active ingredient comprises therapeutically effective dose of vitamin A, or a pharmaceutically acceptable isomer, analog, and/or derivative thereof, or a mixture thereof. This dry powder is then milled into a fine aerosolizable powder, using equipment and processing steps known in the art of aerosol drug product production, and the resulting powder can be dosed by inhalation. In some embodiments, the powder can be dosed as an aerosol in a gas comprising oxygen. In some embodiments, the gas can comprise oxygen in higher levels than in ambient air (20.8-21.0 %v/v). By way of a non-limiting example, a disclosed pharmaceutical composition can be dried, if necessary, mixed with a pharmaceutically acceptable preparation of dextrose, and then milled. The resulting preparation can be aerosolized by forcing out of a syringe and then inhaled (Raleigh SM, Verschoyle RD, et al, British Journal of Cancer (2000) 83(7), 935-940).

### 2) Antibiotics

One embodiment of the present disclosure is to also include one or more antibiotic compounds in the formulation. Inclusion in formulations meant for dosing by inhalation would be especially useful for infections of the respiratory tract, with such dosing placing both the antibiotic and the insoluble active ingredient directly at the site of the infection. Infections residing in the sinus, bronchi, or other upper-respiratory sites, or in the alveoli or other lower-respiratory sites, among others, would be targets for such treatment. While not being limited to any particular theory, it is believed that the insoluble active ingredient would stimulate one or more beneficial biological responses, as disclosed herein, while the antibiotic would be delivered essentially in direct contact with the causative infectious agent that the antibiotic is supposed to attack. Non-limiting examples of classes of antibiotics relevant for such embodiments include, for example, penicillins, tetracyclines, cephalosporins, quinolones, lincomycins, macrolides, sulfonamides, glycopeptides, aminoglycosides, and carbapenems, among other known classes. Non-limiting examples of insoluble antibiotics include, for the purposes of this disclosure, tetracyclines, hydrophobic members of the cephalosporin family, quinolones, macrolides, telavancin, sulfamethoxazole, rifampin, clofazimine, bedaquiline, and dalifopristin. Non-limiting examples of soluble antibiotics include, for the purposes of this disclosure, penicillins, hydrophilic members of the cephalosporin family, lincomycins, sulfonamides, glycopeptides, aminoglycosides, carbapenems, clindamycins, daptomycin, doxycycline, linezolid, minocycline, quinupristin, tigecycline, trimethoprim, vancomycin, isoniazid, ethambutol, and pyrazinamide. The antibiotics included in the disclosed pharmaceutical composition may be used singly or in combination and may be selected for known effects against diagnosed infectious agents. Of specific interest are antibiotics or classes of antibiotics used to treat methicillin-resistant Staphylococcus aureus infections, for example, but not limited to, clindamycin, daptomycin, doxycycline, linezolid, minocycline, quinupristin, dalifopristin, telavancin, tigecycline, trimethoprim, sulfamethoxazole, vancomycin, and the like. These antibiotics or classes of antibiotics may be used alone or in combination. Also of relevance are compounds used to treat mycobacterial infections, which are the causes of diseases including but not limited to tuberculosis, leprosy, digestive or dermal ulcers, among other diseases. Relevant antibiotics and related compounds include, but are not limited to, isoniazid, rifampin, ethambutol, pyrazinamide, clofazimine, bedaquiline and linezolid, and the like, dosed either alone or in combination.

### 3) Anti-Viral Compounds

Another preferred embodiment of the present disclosure includes, but is not limited to, one or more anti-viral compounds in the formulation. Without being limited to any particular theory, it is envisaged that doses would deliver both the insoluble active ingredient and one or more anti-viral compounds directly into the lung, providing especially advantageous for delivery of these one or more anti-viral compounds directly to the site of the infection in the case of viral lung infection. By way of a non-limiting example, medications specifically designed to mitigate influenza infection are of particular utility, including, but not limited to, oseltamivir, zanamivir, peramivir, and the like. Other medications that mitigate the severity of respiratory syncytial virus (RSV), include, but are not limited to, ribavirin. Non-limiting examples of an insoluble antivirals include remdesivir, peramvir, and hydrophobic enzyme inhibitors. Non-limiting examples of soluble antivirals include oseltamivir phosphate, zanamivir, ribavirin, nucleoside analogs, interferons, inhibitors of a viral protease, inhibitors of viral reverse transcription, inhibitors of viral polymerases, and inhibitors of neuraminidase. Particularly preferred are compounds meant to minimize the impact of coronavirus infections, with a particular focus on SARS-CoV-2 infections, with compounds like, but not limited to, remdesivir having been in the news of late. Similarly, compounds that have been tested for treating SARS (severe acute respiratory syndrome) and MERS (Middle East respiratory syndrome) infections, caused by other coronaviruses, that were the subject of widespread outbreaks in the past several decades, including, but not limited to, nucleoside analogs, interferons, protease inhibitors, reverse transcriptase inhibitors, and neuraminidase inhibitors may be incorporated into the disclosed pharmaceutical compositions (as tested and reported on, for example (Emily L.C. Tan et al., Inhibition of SARS Coronavirus Infection In Vitro with Clinically Approved Antiviral Drugs, 10 EMERGING INFECTIOUS DISEASES 581-586 (2004))).

### 4) Co-Dosing With Other Vitamins

An embodiment of the present disclosure is a combination of an insoluble active ingredient together with an insoluble vitamin. In some embodiments, the insoluble vitamin is vitamin D, a pharmaceutically acceptable isomer, analog, and/or derivative thereof, or a mixture thereof, Vitamin E, a pharmaceutically acceptable isomer, analog, and/or derivative thereof, or a mixture thereof, and/or Vitamin K, a pharmaceutically acceptable isomer, analog, and/or derivative thereof, or a mixture thereof. The insoluble vitamin may be present singly or in combination. Vitamin D is known to act in concert with vitamin A to stimulate receptor proteins in the Retinoic Acid Receptor (so-called RAR) and/or Retinoid X Receptor (RXR) families, which initiate biochemical processes beneficial to lung healing and maturation (Sneha K. Taylor et al., Inhaled Vitamin D: A Novel Strategy to Enhance Neonatal Lung Maturation, 194 LUNG 931-943 (2016)) and/or macrophage-mediated phagocytic destruction of infectious agents (Anand et al.). Vitamin E is also known to have a stimulatory effect in combatting infection (Ibrahim et al.). Vitamin K is known to be depleted during pneumonia, and is similarly hypothesized to be depleted during SARS-CoV-2 infection, leading to damage to the critical function of elastic protein fibers in lung (Janssen R, et al., “Vitamin K metabolism as the potential missing link between lung damage and thromboembolism in Coronavirus disease 2019” (2020) British J Nutrition, First View, pp. 1 - 8, DOI: https://doi.org/10.1017/S0007114520003979).

### 5) Particle Size and Distribution

The therapeutic effect of aerosolized, vaporized, and/or nebulized therapies, dosed by inhalation, is dependent upon the dose deposited in the lung from the action of inhalation and its distribution inside the body, particularly in the lung. Aerosol solid particle or liquid droplet size is one of the most important variables in defining the dose deposited and the distribution of drug aerosol in the lung. In the case of liquids meant to be dosed by inhalation, solid particle or liquid droplet size refers to the size of the constituent droplets in the vapor or mist that is generated by, for example, a nebulizer.

Generally, inhaled particles are subject to deposition by one of two mechanisms: impaction, which usually predominates for larger solid particles or liquid droplets, and sedimentation, which is prevalent for smaller solid particles or liquid droplets. Impaction occurs when the momentum of an inhaled solid particle or liquid droplet is large enough that the solid particle or liquid droplet does not follow the air stream and encounters a physiological surface. In contrast, sedimentation occurs primarily in the lower lung when very small solid particles or liquid droplets which have traveled with the inhaled air stream encounter physiological surfaces as a result of gravitational settling.

Pulmonary drug delivery may be accomplished by inhalation of an aerosol through the mouth and throat. Aerosolized solid particles or liquid droplets having an aerodynamic diameter of greater than about 5 µm generally do not reach the lung; instead, they tend to impact the back of the throat and are swallowed and possibly orally absorbed. Solid particles or liquid droplets having diameters of about 3 to about 5 µm are small enough to reach the upper- to mid-pulmonary region (conducting airways), although currently some practitioners believe this size may be less likely to reach the alveoli. Disclosed are solid particles or liquid droplets having diameters of about 3 to about 5 µm that may reach the alveoli. Non-limiting examples of solid particle or liquid droplet diameters include 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0 µm. Ranges with lower and upper values having any of the disclosed solid particle or liquid droplet diameters are disclosed with the proviso that the upper value is greater than the lower value. The diameters may be mass median diameters, volume median diameters, and mass median aerodynamic diameters. Smaller solid particles or liquid droplets, i.e. about 0.5 to about 3 µm, are capable of reaching the alveolar region. Non-limiting examples of solid particle or liquid droplet diameters include 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0 µm. Ranges with lower and upper values having any of the disclosed solid particle or liquid droplet diameters are disclosed with the proviso that the upper value is greater than the lower value. The diameters may be mass median diameters, volume median diameters, and mass median aerodynamic diameters. Solid particles or liquid droplets having diameters smaller than about 0.5 µm tend to be exhaled during tidal breathing but can also be deposited in the alveolar region by a breath hold.

Aerosols used in pulmonary drug delivery are made up of a wide range of solid particle or liquid droplet sizes, so statistical descriptors are used. Aerosols used in pulmonary drug delivery are typically described by their mass median diameter (MMD), that is, half of the mass is contained in solid particles or liquid droplets larger than the MMD, and half the mass is contained in solid particles or liquid droplets smaller than the MMD. For solid particles or liquid droplets with uniform density, the volume median diameter (VMD) can be used interchangeably with the MMD. Determinations of the VMD and MMD may be made by laser diffraction. The extent of solid particle or liquid droplet size variation from the average measured VMD and MMD may be described by the geometric standard deviation (GSD). However, the deposition of a solid particle or liquid droplet in the respiratory tract is more accurately described by the solid particle’s or liquid droplet’s aerodynamic diameter, thus, the mass median aerodynamic diameter (MMAD) is typically used. MMAD determinations may be made by inertial impaction, time of flight, or laser diffraction measurements. For aqueous solid particles or liquid droplets, the VMD, MMD, and MMAD may typically be substantially the same. Humidity can be most commonly by performing measurements inside a closed controlled environmental changer. However, if humidity is not controlled as the aerosol transits the impactor, MMAD determinations may be smaller than MMD and VMD due to dehydration. For the purposes of this disclosure, VMD, MMD and MMAD measurements are considered to be under controlled conditions such that descriptions of VMD, MMD and MMAD will be substantially comparable. Nonetheless, for the purpose of this disclosure, the solid particle or liquid droplet size of the aerosol solid particles or liquid droplets will be given as MMAD as may be determined by measurement at room temperature with a Next Generation Impactor (NGI) in accordance with US Pharmacopeial Convention in Process Revision <601> Aerosols, Nasal Sprays, Metered-Dose Inhalers, and Dry Powder Inhalers, Pharmacopeial Forum (2003), Volume Number 29, pages 1176-1210 and also disclosed in Jolyon Mitchell, Mark Nagel “Particle Size Analysis of Aerosols from Medicinal Inhalers”, KONA Powder and Particle Journal (2004), Volume 22, pages 32-65.

In accordance with the present disclosure, the solid particle or liquid droplet size of the aerosol may be optimized to maximize the deposition of the insoluble active ingredient to maximize therapeutic effect and tolerability. Aerosol solid particle or liquid droplet size may be expressed in terms of the mass median aerodynamic diameter (MMAD). Large particles (e.g., MMAD > 5 µm) tend to deposit in the extrathoracic and upper airways because they are too large to navigate bends in the airways. Intolerability (e.g., cough and bronchospasm) may occur from upper airway deposition of large particles.

Thus, in accordance with a preferred embodiment, the MMAD of the aerosol may be less than about 15 µm, preferably less than about 5 µm, more preferably less than 3 µm. In a particularly preferred embodiment, the MMAD of the aerosol may be 15, 14, 13, 12, 11, 10, 9, 8, 7.75, 7.5, 7.25, 7.0, 6.75, 6.5, 6.25, 6.0, 5.75, 5.5, 5.25, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, and 1.0 µm. Ranges with lower and upper values having any of the disclosed MMAD values are disclosed with the proviso that the upper value is greater than the lower value.

### 6) Aerosol Generators

For aqueous and other non-pressurized liquid systems, a variety of aerosol vapor generators are available to aerosolize the compositions of the present disclosure. The disclosed devices may be aerosolizers, nebulizers, vaporizers or the like, and can also include designs adapted for function on large or small volumes of input material. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., DeVilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Heathcare, Inc. and DeVilbiss Health Care, Inc. Vibrating mesh nebulizers rely upon either piezoelectric or mechanical pulses to respirable liquid droplets generate. Other examples of nebulizers for use with the compositions of the present disclosure described herein are described in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922;6,557,549; and 6,612,303. Commercial examples of nebulizers that can be used with the compositions described herein include Respirgard II®, Aeroneb®, Aeroneb® Pro, and Aeroneb® Go produced by Aerogen; AERx® and AERx Essence™ produced by Aradigm; Porta-Neb®, Freeway Freedom™, Sidestream, Ventstream and I-neb produced by Respironics, Inc.; and PARI LCPlus®, PARI LC-Star®, and e-Flow7m produced by PARI, GmbH. Generic versions of these devices are also disclosed. Further non-limiting examples are disclosed in U.S. Pat. No. 6,196,219.

Other nebulizing technologies disclosed in US 5,603,314; 5,611,332; 6,230,703 and 5,630,409; are capable of both fast generation of aerosol from liquid and while also yielding, desirably, very small aerosolized particles ideal for alveolar deposition upon inhalation. A commercial example of this nebulizer technology is the Swirler® Radioaerosol System from AMICI, Inc (Spring City, Pennsylvania) although generic devices are also disclosed. Values for particle diameter, MMD, VMD, and MMAD that the Swirler is capable of have been shown to be less than 1 micron (see, for example, http://www.amici-inc.com/pdf/Swirler%20Technology%20Brief%20for%20web-site.pdf). In some embodiments, these values can be 1.10, 1.00, 0.90, 0.80, 0.70, 0.60. 0.50, 0.40, 0.30, 0.20, 0.10, 0.09, 0.08, 0.07, 0.06, 0.05, 0.04, 0.03, 0.02, or 0.01 microns. Ranges with lower and upper values having any of the disclosed particle diameters, MMD, VMD, and MMAD are disclosed with the proviso that the upper value is greater than the lower value. By way of non-limiting examples, ranges of particle diameters include 0.65-1.1 and 0.43-0.65 microns. The Swirler® technology possesses a unique capacity for nebulizing aqueous solutions, as the other devices above, and additionally can produce fine particles of appropriate respirable size, as described herein, from oils and liquids more viscous than typical aqueous solutions, and from dry or lyophilized powders, and may be useful for embodiments of the present disclosure where the formulation may be an oil and/or be more viscous than water.

In accordance with the present disclosure, the pharmaceutical composition may be aerosolized using a nebulizing device selected from an ultrasound nebulizer, an electron spray nebulizer, a vibrating membrane nebulizer, a jet nebulizer, or a mechanical soft mist nebulizer.

In a further embodiment, the disclosed device controls the patient’s inhalation flow rate either by an electrical or mechanical process.

In another embodiment, the aerosol production by the device is triggered by the patient’s inhalation, such as with an AKITA® device.

Examples of the nebulizers/devices that may be used in accordance with the present invention include, but are not limited to, Vectura Fox®, Pari eFlow®, Pari Trek® S, Philips Innospire mini, Philips InnoSpire Go, Aeroneb® Go, Aerogen® Ultra, Respironics Aeroneb, Akita®, Medspray® Ecomyst®, Respimat®, and AVICI’s Swirler® device.

### 7) Definitions

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments. For any value disclosed herein, a number “about” that value is also disclosed.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

By “pharmaceutically acceptable acid” is meant acids which are not biologically or otherwise undesirable in pharmaceutical compositions of the present disclosure. Pharmaceutically acceptable acids include, but are not limited to, inorganic acids, for example, hydrochloric acid, hydrobromic acid, sulfuric acid, nitric acid, phosphoric acid and the like, or organic acids, for example acetic acid, oleic acid, palmitic acid, stearic acid, glycolic acid, pyruvic acid, oxalic acid, maleic acid, malonic acid, succinic acid, fumaric acid, tartaric acid, citric acid, ascorbic acid, lactic acid, tartaric acid, and the like. Conjugate bases of the disclosed pharmaceutically acceptable acids are also disclosed.

By “pharmaceutically acceptable base” is meant bases which are not biologically or otherwise undesirable in pharmaceutical compositions of the present disclosure. Pharmaceutically acceptable bases include sodium hydroxide, ammonium hydroxide, potassium hydroxide, histidine, arginine, and lysine. Conjugate acids of the disclosed pharmaceutically acceptable bases are also disclosed.

By “vitamin A, a pharmaceutically acceptable isomer, analog, and/or derivative thereof, or a mixture thereof,” is meant versions of vitamin A that retain desirable pharmacological activity and that are not biologically or otherwise undesirable in pharmaceutical compositions of the present disclosure.

By pharmaceutically acceptable carrier is meant those carriers which are not biologically or otherwise undesirable in pharmaceutical compositions of the present disclosure.

By “therapeutically effective amount”, “therapeutically effective dose”, or “pharmaceutically effective amount” is meant an amount of an insoluble active ingredient that has a therapeutic effect. The “therapeutically effective amount”, “therapeutically effective dose”, or “pharmaceutically effective amount” may be an amount that prevents, relieves, or treats to some extent one or more symptoms of a vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS).

The doses of insoluble active ingredient that are useful in treatment or prevention of a vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS) may be in therapeutically effective amounts. Thus, as used herein, a “therapeutically effective amount” may mean an amount of an insoluble active ingredient that produces a desired therapeutic effect as judged by testing in human and animal subjects, clinical trial results, and/or model animal studies, or which has been proven to be effective in routine medical practice to benefit a subject.

The amount of the insoluble active ingredient, the frequency of administration such as but not limited to daily dose, and the length of time for a given course of treatment can be routinely determined by one of skill in the art, and will vary, depending on several factors, such as but not limited to the patient’s height, weight, sex, age and medical history. The identity of the infectious agent and/or the severity of the symptoms of an infected patient may also be important factors in determining the effective dose. For prophylactic treatments, an effective dose is that amount that would be effective to prevent a vitamin deficiency, disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS). For prophylactic treatments, the therapeutically effective amount may also be that amount that would be effective to elicit an immune response to exposure to active bacteria or virus and/or to a vaccination meant to promote immunity against the bacteria or virus.

“Hyperoxia” is defined as a condition or intervention coming from exposure of lungs to oxygen levels greater than that of the normal environment. For humans and animals, normal oxygen levels are those in ambient air (typically 20.8-21.0% oxygen), with exposure to these normal levels beginning at the moment of birth upon full-term development.

Regarding prematurely-born neonates, “hyperoxia” or “hyperoxic conditions” are used herein to refer to conditions in which lung tissue is exposed to oxygen levels under normal conditions prematurely or oxygen-enriched air to support normal blood oxygen levels.

The term “between” in connection with a range of values is intended to encompass the upper and lower limits of these values as well as any value falling between these limits. By way of a non-limiting example, if a pharmaceutical composition comprises “between 0.03% (w/w) and 4.0% (w/w) of vitamin A,” the pharmaceutical composition may comprise 0.03% (w/w) vitamin A, 4.0% (w/w) vitamin A, or any amount of vitamin A falling between 0.03% (w/w) and 4.0% (w/w).

“Treat”, “treatment”, or “treating” as used herein refers to administering a pharmaceutical composition for prophylactic and/or therapeutic purposes.

The term “prophylactic treatment” or “prophylaxis” refers to treating a patient who does not have symptoms of a condition or conditions caused by vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS) but who is susceptible to, or otherwise at risk of such condition or conditions.

The terms “prophylactic treatment” or “prophylaxis” can refer to several different goals associated with administration of the disclosed pharmaceutical composition. In the case of a patient who is not currently nor ever has been infected, the terms “prophylactic treatment” or “prophylaxis” can mean treatment with a pharmaceutical formulation meant to improve the efficacy of a vaccination administered to elicit immunity to the infectious agent. It can also mean boosting the ability of the subject to avoid contracting an infection, regardless of whether or not vaccination is attempted. Alternatively, in the case of a patient who may be known to be infected with a bacteria and/or a virus but is not yet showing one or more deleterious symptoms known to be associated with the infection, the treatment may be used to prevent symptoms or mitigate the severity of those symptoms should they start to manifest. The term “therapeutic treatment” refers to administering treatment to a patient already suffering from a condition or conditions caused by vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS). Thus, in preferred embodiments, treating is the administration to a mammal (either for therapeutic or prophylactic purposes) of therapeutically effective amounts of vitamin A, or a pharmaceutically acceptable isomer, analog, and/or derivative thereof. In some embodiments, the prophylaxis may be temporary, including but not limited to 1 day, 2 days, 3 days, 4 days, 5 days, 6 days, 7 days, 8 days, 9 days, 10 days, 11 days, 12 days, 13 days, 14 days, a week, 2 weeks, 3 weeks, 4 weeks, 5 weeks, 6 weeks, 7 weeks, 8 weeks, 9 weeks, 10 weeks, a month, 2 months, 3 months, 4 months, 5 months, 6 months, 7 months, 8 months, 9 months, 10 months, 11 months, 12 months, 13 months, 14 months, 15 months, 16 months, 17 months, 18 months, a year, 2 years, 3 years, four years, five years, six years, 7 years, 8 years, 9 years, or 10 years.

By “homogeneous intermediate state” is meant a mixture at or near the water-in-oil or oil-in-water inversion point.

The term “substantially all” refers to an amount of 95% or greater.

The term “patient” as used herein refers to a mammal, preferably a human adult, child, neonate, or prematurely born neonate, or an animal in need of the prophylaxis or treatment as disclosed herein, including humans, be they an adult, child, neonate, or prematurely born neonate, or other animals including livestock or pets. The livestock can be a horse, a cow, a sheep, or a pig and pets may be cats, dogs, rabbits, and ferrets.

Unless otherwise stated, the term “inhalation” is meant to refer to oral or nasal entry into the lungs.

Unless otherwise stated, a “healthy normal” physiological state is that state found in a subject not afflicted with a disease or disorder. By way of a non-limiting example, a physiological state can be lung maturation status.

Unless otherwise stated, a “healthy normal” level of a protein is that amount or distribution found in a subject not afflicted with a disease or disorder. By way of a non-limiting example, a protein can be peroxisome proliferator-activated receptor gamma.

Unless otherwise stated, a composition of matter is “in aerosol form” when the composition of matter is a suspension of fine solid particles or fine liquid droplets in a gas.

Unless otherwise stated, a composition of matter “in liquid aerosol form” when the composition of matter is disposed within a fine liquid droplet that is in suspension within a gas. In some embodiments, the composition of matter is a solid particle disposed within a liquid droplet.

Unless otherwise stated, a composition of matter is “in solid aerosol form” when the composition of matter is a solid particle in a gas with the proviso that the solid particle is not disposed within a liquid droplet.

Unless otherwise stated, an “active ingredient” means any component that is intended to furnish pharmacological activity or other direct effect in the diagnosis, cure, mitigation, treatment, or prevention of disease, or to affect the structure or any function of the body of humans. The term includes those components that may undergo chemical change in the manufacture of the drug product and be present in the drug product in a modified form intended to furnish the specified activity or effect.

Unless otherwise stated, an “inactive ingredient” means any component other than an active ingredient.

Unless expressly stated otherwise, all percentages are weight percent, relative to the total weight of the pharmaceutical composition according to this disclosure.

Unless expressly stated otherwise, all values in mL and L refer to the total volume of the pharmaceutical composition according to this disclosure.

Unless expressly stated otherwise, parameters and conditions (such as temperature, pressure, relative humidity, volume, weight, concentration, pH value, titration acidity, capacity of buffer system, osmolarity, content of molecular oxygen, storage stability, color, and the like) are determined and measured in accordance with the requirements and recommendations as set forth in the European Pharmacopoeia (Ph. Eur.). Unless expressly stated otherwise, all references to Ph. Eur. refer to the version that is officially valid in September 2016. General conditions are typically ambient conditions.

By “parenteral administration” is meant routes of administration known to those skilled in the art, and include subcutaneous administration, intraperitoneal administration, intravenous administration, intradermal administration, and intramuscular administration.

By “suitable for parenteral administration” is meant that the pharmaceutical composition of the present disclosure meets quality standards known to those skilled in the art as found, for example, in the United States Pharmacopeia, the European Pharmacopeia, and the Japanese Pharmacopeia. Such standards include, for example that the composition by sterile and pyrogen-free, that it be clear, or practically exempt of visible particles, and also free of sub-visible particles as required by these pharmacopeias, and that there is no evidence of phase separation or aggregate formation.

Unless otherwise stated, a “non-natural surfactant” is a surfactant not found in nature.

Unless otherwise stated, a pharmaceutical composition “consisting essentially of” listed ingredients may include unlisted ingredients that do not materially affect the basic and novel properties of the pharmaceutical composition. Non-limiting examples of ingredients that do not materially affect the basic and novel properties of the pharmaceutical composition include salts, chemicals other than chlorobutanol added as preservatives that retard biological contamination or provide stabilization of the key pharmaceutical constituent, or to control, for example, pH or other typical chemical properties. Examples of salts include, but are not limited to, sodium chloride, nitrite salts, sulfite salts, bisulfite salts, and metabisulfite salts. Examples of preservatives include, but are not limited to, EDTA, benzyl alcohols, benzalkonium chloride, phenol, nitrite salts, sulfite salts, bisulfite salts, metabisulfite salts, tocopherol, ascorbic acid, citric acid, butylated hydroxyanisole, butylated hydroxytoluene, and the like. Examples of controllers of pH or other typical chemical properties include, but are not limited to, inorganic acids such as hydrochloric acid, hydrobromic acid, sulfuric acid, nitric acid, phosphoric acid and the like, or organic acids, such as acetic acid, oleic acid, palmitic acid, stearic acid, glycolic acid, pyruvic acid, oxalic acid, maleic acid, malonic acid, succinic acid, fumaric acid, tartaric acid, citric acid, ascorbic acid, lactic acid, tartaric acid and the like, or bases such as sodium hydroxide, ammonium hydroxide, potassium hydroxide, histidine, arginine and lysine. Any controller of pH or other typical chemical properties disclosed herein can be used alone or in combination with other such controllers.

In some embodiments, the pharmaceutical composition does not comprise a preservative. It has been suggested that chlorobutanol should not be used as a preservative in injectable preparations intended for neonates and children (see, for example, Pharmacy in Practice, May 2004, p. 101). Similar considerations apply for the disclosed pharmaceutical compositions in aerosol form. Chlorobutanol has been implicated in producing somnolence in patients given high doses of salicylamide or morphine infusions when used as a preservative (see, for example, Borody, T. et al., Chlorbutanol toxicity and dependence, Med J Aust 1979; 1: 288; and DeChristoforo, R., et al., High-dose morphine infusion complicated by chlorobutanol-induced somnolence, Annals of Internal Medicine 1983; 98; 335-6). A delayed cellular type of hypersensitivity reaction to chlorobutanol used to preserve heparin administered by subcutaneous injection has also been reported (see, for example, Dux, S., et al., Hypersensitivity reaction to chlorbutanol-preserved heparin, Lancet 1981; 1: 149). Benzalkonium chloride (BAC) is a known bronchoconstrictor, with use in aerosols for treating asthma, sometimes in combination with EDTA, having been shown to induce paradoxical bronchoconstriction (see, for example, Beasley R, et al., “Preservatives in nebulizer solutions: risks without benefit,” Pharmacotherapy, .Jan-Feb 1998;18(1):130-9); and as recently as 2020 has been reported as leading to longer drug use and/or additional respiratory support measures for treating hospitalized asthmatic childrenl (Pertzborn MC, et al., “Continuous Albuterol With Benzalkonium in Children Hospitalized With Severe Asthma,” PEDIATRICS 145:4, April 2020:e20190107).

Unless otherwise stated, a pharmaceutical composition may be “adapted for inhalation.” Non-limiting examples of such pharmaceutical compositions are pharmaceutical compositions diluted with an appropriate medium, including, for by way of a non-limiting example, sterile water or saline solution, to be more compatible with liquid nebulizer devices, addition of saline or adjustment of pH to be more compatible for lung tissue exposure upon inhalation. The pharmaceutical composition may also be adapted for inhalation by disposing the pharmaceutical composition in a container suitable for loading into a nebulizer.

A “particle” may refer to a solid or a liquid. Liquid particles may also be referred to as droplets. Solid particles may also be disposed inside liquid droplets.

A “conducting zone” is a portion of the lung that does not exchange oxygen. The bronchus is a non-limiting example of a conducting zone.

A “respiratory zone” is a portion of the lung that exchanges oxygen. An alveolus is a non-limiting example of a respiratory zone.

“Ambient temperature” is between 20 and 24° C.

A substance is “insoluble” if the substance does not substantially dissolve in water after one hour of stirring at ambient temperature and pressure.

An insoluble active ingredient is “at least partially solubilized with a surfactant” when the presence of the surfactant renders soluble at least one percent of the insoluble active ingredient.

In some embodiments, the insoluble active ingredient is the only active ingredient in the pharmaceutical composition.

In some embodiments, the pharmaceutical composition comprises an insoluble active ingredient and one or more additional insoluble active ingredients.

### 8) Use in Treatment and/or Prophylaxis

The pharmaceutical compositions according to the present disclosure may be intended for the use in the treatment and/or prophylaxis of a vitamin deficiency disorder disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS). Vitamin A deficiency disorders include, but are not limited to, bronchopulmonary dysplasia, retinopathy of prematurity, and nyctalopia. Infections can include, but are not limited to, neonatal sepsis, hospital acquired sepsis, sepsis from premature rupture of membranes, measles, meningitis, pneumonia, necrotizing enterocolitis, and other viral or bacterial infections (see, for example, Wiseman EM et al., “The vicious cycle of vitamin a deficiency: A review.” Crit Rev Food Sci Nutr. 2017 Nov 22;57(17):3703-3714. Hyperoxia damage can include, but are not limited to, iatrogenic causes, or industrial or workplace accidents. The pharmaceutical compositions according to the present disclosure may be intended for the use in the treatment and/or prophylaxis of a bacterial infection or a viral infection. By infection is meant infections of bacterial or viral origin, either singly or multiple simultaneously, which may be affecting one or more locations, tissues, or organs in the body, up to or including the entire body.

By way of a non-limiting example, the amount of vitamin A, or an isomer, analog, and/or derivative thereof, or a mixture thereof may be expressed USP units, international units, as a weight, or a molar content of vitamin A or an isomer, analog, and/or derivative thereof, or a mixture thereof. One USP unit is equivalent to one international unit and is equivalent to 0.3 mcg of retinol.

### 10) Micelles

By ‘micelle’ is meant an assembly of molecules, generally of the class known as detergents, surfactants, or similar amphipathic molecules having both hydrophobic and hydrophilic characteristics, which form into a sphere or other compact shapes with the outer surface being comprised by a monolayer of the detergent molecules, which in aqueous solutions form with the hydrophilic portions facing outward and the hydrophobic portions facing inward. The hydrophilic portions of the detergent interact with water, facilitating a situation in which the micelles are stably dispersed or dissolved in aqueous media. The hydrophobic core of micelles is useful for interacting with other hydrophobic molecules, providing a hydrophobic environment within which these other hydrophobic molecules, as in the case with the insoluble active ingredient can be fat-dissolved inside the micelles, and because of the hydrophilic surface of the micelles, these otherwise water-insoluble hydrophobic molecules are facilitated to be miscible within aqueous solutions.

Micelles are typically small, and when they are generally of similar density as water, they can remain dissolved indefinitely. Such permanent miscibility is a preferred feature of the pharmaceutical compositions of the present disclosure.

Micelle size may be determined by methods known in the art. Visual inspection can be used, in the first instance, to determine visual clarity. Quantitation of light scattering, for example at 400 nm, and by dynamic light scattering (DLS) may be used to directly assess micelle radii and size distribution. Typically micelles will be of radii or diameter including, but not limited to, 1,000 nm, 500 nm, 100 nm, 50 nm, 10 nm, 5 nm or 1 nm, with the smaller range of 100 nm and lower being ideal for compatibility with most nebulizer technologies. In some embodiments, the average micelle radius or diameter is 1,000, 900, 800, 700, 600, 500, 450, 400, 350, 300, 250, 200, 150, 100, 90, 80, 70, 75, 60, 50, 45, 40, 35, 30, 25, 20, 19, 18, 17, 16, 15, 14,13, 12, 11, 10, 9.5, 9.0, 8.5, 8.0, 7.5, 7.0, 6.5, 6.0, 5.5, 5.0, 4.5, 4.0, 3.5, 3.0, 2.5, 2.0, 1.5, or 1.0 nm. Ranges with lower and upper values having any of the disclosed average micelle radius or diameter with the proviso that the upper value is greater than the lower value. In some embodiments, the average micelle radii or size is 5-10, 100-150, or 500-1,000 nm.

Accordingly, in one embodiment of the present disclosure, a pharmaceutical composition suitable for inhalation is provided, comprising between 0.03% (w/w) and 4.0% (w/w) of vitamin A, or a pharmaceutically acceptable isomer, analog, and/or derivative thereof, or a mixture thereof, a weight of surfactant which is between 4.0 times and 5.0 times the weight of the vitamin A, or the pharmaceutically acceptable isomer, analog, and/or derivative thereof, or mixture thereof, contained in the composition, wherein the remainder of the composition comprises water and, optionally, wherein the pH has been adjusted by addition of a pharmaceutically acceptable acid and/or pharmaceutically acceptable base.

In another embodiment of the present disclosure, a pharmaceutical composition is provided, comprising between 0.03% (w/w) and 4.0% (w/w) of all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, CD437 or CD1530, or a mixture thereof, a weight of surfactant which is between 4.0 times and 5.0 times the weight of the all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, CD437, or CD-1530, or intrinsically fluorescent retinol analogs such as 4-(5,5,8,8-tetramethyl-5,6,7,8-tetrahydronaphthalen-2-ylethynyl)benzoic acid (also known by the acronym EC23; Christie VB, et al. “Synthesis and evaluation of synthetic retinoid derivatives as inducers of stem cell differentiation.” Organic & biomolecular chemistry 6.19 (2008): 3497-3507) and related compounds (as described in, for example, Chisholm et al. “Fluorescent Retinoic Acid Analogues as Probes for Biochemical and Intracellular Characterization of Retinoid Signaling Pathways”, ACS Chem. Biol. 2019, 14, 3, 369-377 or the mixture thereof, contained in the composition, wherein the remainder of the composition comprises water and, optionally, wherein the pH has been adjusted by addition of a pharmaceutically acceptable acid and/or pharmaceutically acceptable base.

In another embodiment of the present disclosure, a pharmaceutical composition is provided, comprising between 0.03% (w/w) and 4.0% (w/w) of all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, CD437, or CD1530, or intrinsically fluorescent retinol analogs such as EC23 and related compounds, a weight of surfactant which is between 4.0 times and 5.0 times the weight of the all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol contained in the composition, wherein the remainder of the composition comprises water and, optionally, wherein the pH has been adjusted to between by addition of a pharmaceutically acceptable acid and/or pharmaceutically acceptable base.

In another embodiment of the present disclosure, a pharmaceutical composition is provided, comprising between 0.03% (w/w) and 4.0% (w/w) of all trans retinol, or the palmitate, acetate, or maleate ester of all trans retinol, a weight of surfactant which is between 4.0 times and 5.0 times the weight of the all trans retinol, or the palmitate, acetate, or maleate ester of all trans retinol contained in the composition, wherein the remainder of the composition comprises water and, optionally, wherein the pH has been adjusted by addition of a pharmaceutically acceptable acid and/or pharmaceutically acceptable base.

In another embodiment of the present disclosure, a pharmaceutical composition suitable for inhalation, parenteral administration, or oral administration is provided, comprising between 0.03% (w/w) and 4.0% (w/w) vitamin A, or an isomer, analog, and/or derivative thereof, or a mixture thereof, a weight of a surfactant which is between 4.0 times and 5.0 times the weight of the vitamin A or an isomer, analog, and/or derivative thereof, or a mixture thereof contained in the composition, wherein the remainder of the composition comprises water, and, optionally, wherein the pH has been adjusted by addition of a pharmaceutically acceptable acid and/or a pharmaceutically acceptable base.

In some embodiments, the weight of the surfactant which is 4.0, 4.1, 4.2, 4.25, 4.3, 4.4, 4.5, 4.6, 4.7, 4.75, 4.8, 4.9, and 5.0 times the weight of the vitamin A or an isomer, analog, and/or derivative thereof, or a mixture thereof. Ranges with lower and upper values having any of the disclosed weights are disclosed with the proviso that the upper value is greater than the lower value.

In another embodiment of the present disclosure, a pharmaceutical composition is provided, wherein the pH has been optionally adjusted to between pH 7.0 and pH 7.5.

In some embodiments, the pharmaceutical composition has a pH value of 7.0, 7.1, 7.2, 7.25, 7.3, 7.4, and 7.5. Ranges with lower and upper values having any of the disclosed pH values are disclosed with the proviso that the upper value is greater than the lower value.

In another embodiment of the present disclosure, a pharmaceutical composition is provided, wherein the surfactant is selected from polysorbate 20, polysorbate 60, polysorbate 80, stearyl alcohol, a polyethylene glycol derivative of hydrogenated castor oil, a polyethylene glycol derivative of hydrogenated castor oil, sorbitan monolaurate, sorbitan monopalmitate, sorbitan monostearate, polyoxyethylene (20) oleyl ether, polyoxyethylene (20) cetyl ether, polyoxyethylene (10) cetyl ether, polyoxyethylene (10) oleyl ether, polyoxyethylene (100) stearyl ether, polyoxyethylene (10) stearyl ether, polyoxyethylene (20) stearyl ether, polyoxyethylene (4) lauryl ether, polyoxyethylene (20) cetyl ether, polyoxyethylene (2) cetyl ether, caprylocaproyl polyoxyl-8 glyceride, polyethylene glycol (20) stearate, polyethylene glycol (40) stearate, polyethylene glycol, polyethylene glycol (8) stearate, polyoxyl 40 stearate, poloxamer 188, polaxamer 312, and mixtures thereof.

In another embodiment of the present disclosure, a pharmaceutical composition is provided, wherein the surfactant is polysorbate 80.

In another embodiment of the present disclosure, a pharmaceutical composition is provided, wherein the particles formed by vitamin A or an isomer, analog, and/or derivative thereof, or a mixture thereof and the surfactant are in the configuration of micelles having a diameter of less than or equal to 500 nm.

In another embodiment of the present disclosure, a pharmaceutical composition is provided, wherein the micelles have a diameter of less than or equal to 250 nm.

In another embodiment of the present disclosure, a pharmaceutical composition is provided, wherein the micelles have a diameter of less than or equal to 100 nm.

In another embodiment of the present disclosure, a pharmaceutical composition is provided, comprising between 0.3% and 3.0% vitamin A or an isomer, analog, and/or derivative thereof, or a mixture thereof.

In another embodiment of the present disclosure, a pharmaceutical composition is provided, comprising between 2.5% and 3.0% vitamin A, or an isomer, analog, and/or derivative thereof, or a mixture thereof.

In some embodiments, the vitamin A, or an isomer, analog, and/or derivative thereof, or a mixture thereof is present in the pharmaceutical composition at 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6. 2.7, 2.8, 2.9, 3.0, 3.1, 3.2, 3.3, 3.4, 3.5, 3.6, 3.7, 3.8, 3.9, and 4.0 %. Ranges with lower and upper values having any of the disclosed amounts of vitamin A, or an isomer, analog, and/or derivative thereof, or a mixture thereof are disclosed with the proviso that the upper value is greater than the lower value.

In another embodiment of the present disclosure, a pharmaceutical composition is provided, wherein the surfactant is polysorbate 80, and the fatty acid content of the polysorbate 80 is between 58% and 100% oleic acid.

In another embodiment of the present disclosure, a pharmaceutical composition is provided, wherein the surfactant is polysorbate 80 and the fatty acid content of the polysorbate 80 is between 85% and 100% oleic acid.

In another embodiment of the present disclosure, a pharmaceutical composition is provided, wherein the surfactant is polysorbate 80, and the fatty acid content of the polysorbate 80 is greater than or equal to 98% oleic acid.

In another embodiment of the disclosure, a pharmaceutical composition is provided, wherein the pharmaceutically acceptable acid is selected from hydrochloric acid, hydrobromic acid, sulfuric acid, nitric acid, phosphoric acid, acetic acid, oleic acid, palmitic acid, stearic acid, glycolic acid, pyruvic acid, oxalic acid, maleic acid, malonic acid, succinic acid, fumaric acid, tartaric acid, citric acid, ascorbic acid, lactic acid, and tartaric acid.

In another embodiment of the present disclosure, a pharmaceutical composition is provided, wherein the pharmaceutically acceptable base is selected from sodium hydroxide, ammonium hydroxide, potassium hydroxide, histidine, arginine and lysine.

In another embodiment of the present disclosure, a pharmaceutical composition is provided, wherein the pharmaceutically acceptable acid is citric acid, and the pharmaceutically acceptable base is sodium hydroxide.

In another embodiment of the present disclosure, a pharmaceutical composition is provided for use in the treatment or prophylaxis of a vitamin A deficiency disorder in a patient in need thereof.

In another embodiment of the present disclosure, a pharmaceutical composition is provided, further comprising, Vitamin B, Vitamin C, Vitamin D, Vitamin E, or an analog and/or derivative of Vitamin B, Vitamin C, Vitamin D, Vitamin E, or a mixture thereof.

In another embodiment of the present disclosure, a pharmaceutical composition is provided, wherein the vitamin A deficiency disorder is selected from neonatal sepsis, hospital-acquired sepsis, sepsis from premature rupture of membranes, bronchopulmonary dysplasia, retinopathy of prematurity, measles, meningitis, pneumonia, necrotizing enterocolitis, a viral infection and a bacterial infection, and a combination of such disorders.

In another embodiment of the present disclosure, a pharmaceutical composition is provided, further comprising one or more antibiotic compounds, or mixtures thereof.

In another embodiment of the present disclosure, a pharmaceutical composition is provided, wherein the antibiotic compound or compounds is selected from penicillins, tetracyclines, cephalosporins, quinolones, lincomycins, macrolides, sulfonamides, glycopeptides, aminoglycosides and carbapenems, and mixtures thereof.

In another embodiment of the present disclosure, a pharmaceutical composition is provided, further comprising an anti-viral compound.

In another embodiment of the present disclosure, a pharmaceutical composition for use is provided, wherein the patient is a human born prematurely, or a neonate.

In another embodiment of the present disclosure, a pharmaceutical composition is provided, wherein the anti-viral compound is selected from oseltamivir, zanamivir, peramivir, ribavirin, remdesivir, a nucleoside analog or analogs, an interferon or interferons, a protease inhibitor or inhibitors, a reverse transcriptase inhibitor or inhibitors, and neuramidinidase inhibitor or inhibitors, or a mixture thereof.

In another embodiment of the present disclosure, a pharmaceutical composition for the use is provided, wherein the vitamin A deficiency disorder is bronchopulmonary dysplasia or retinopathy of prematurity.

In another embodiment of the present disclosure, a pharmaceutical composition is provided, wherein such composition is suitable for intravenous or intraarterial parenteral administration, oral administration, or administration by inhalation through the mouth or nose.

In another embodiment of the present disclosure, a pharmaceutical composition for the use is provided, wherein the vitamin A deficiency disorder is bronchopulmonary dysplasia.

In another embodiment of the present disclosure, provided is a system for use in the prophylaxis or treatment of a vitamin A deficiency disorder wherein the system comprises a therapeutically effective dose of a composition and a nebulizer is provided, which produces aerosol droplets of the composition, and wherein the aerosol droplets produced by the disclosed system have a mass median aerodynamic diameter of less than 15 microns.

In another embodiment of the present disclosure, a system for use is provided, wherein the aerosol droplets produced by the disclosed system have a mass median aerodynamic diameter of less than 10 microns.

In another embodiment of the present disclosure, a system for use is provided, wherein the aerosol droplets produced by the disclosed system have a mass median aerodynamic diameter of less than 5 microns.

In another embodiment of the present disclosure, a system is provided, wherein the aerosol droplets produced by the system have a mass median aerodynamic diameter of less than 3 µm.

In some embodiments, the aerosol droplets have a mass median aerodynamic diameter of 15, 14.5, 14.0. 13.5, 13.0. 12.5, 12.0, 11.5, 11.0, 10.5, 10.0, 9.5, 9.0, 8.5, 8.0, 7.5, 7.0, 6.5, 6.0, 5.5, 5.0, 4.75, 4.5, 4.25, 4.0, 3.75, 3.5, 3.25, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5. 0.4, 0.3, 0.2, and 0.1 µm. Ranges with lower and upper values having any of the disclosed mass median aerodynamic diameters are disclosed with the proviso that the upper value is greater than the lower value.

In another embodiment of the present disclosure, a method of achieving or maintaining healthy normal lung maturation status is provided, comprising administration by inhalation of a disclosed composition.

In another embodiment of the present disclosure, a method is provided, wherein the healthy normal lung maturation status comprises one or more favorable characteristics of lung tissue histology or levels of one or more indicators of metabolic status, or a combination thereof.

In another embodiment of the present disclosure, a method is provided, wherein the lung histology characteristic comprises maintenance of healthy levels of alveolar count, alveolar size, or alveolar septal thickness, or a combination thereof.

In another embodiment of the present disclosure, a method is provided where the metabolic status comprises maintenance of healthy levels of Surfactant Protein A, Surfactant Protein B, Surfactant Protein C, Surfactant Protein D, Peroxisome Proliferator-activated Receptor Gamma, BCL-2 Protein, BCL-2 Associated X Protein, Retinoic Acid X Receptor Alpha, Retinoic Acid X Receptor Beta, Retinoic Acid X Receptor Gamma, Vascular Endothelial Growth Factor, T-complex Protein 1 Subunit Alpha (also known as CTP:Phosphocholine Cytidylyltransferase Subunit Alpha), Fetal Liver Kinase 1, Beta-catenin, Activin Receptor-Like Kinase 5, or lung surfactant phospholipid synthesis rate, either singly or in a combination thereof.

In another embodiment of the present disclosure, a method of avoiding damage to normal healthy lung physiology or initiation of biological shock pathways leading to malformation of lung in any manner differing from healthy normal lung physiology that may be initiated by biological or chemical damage to lung tissue is provided, comprising administration by inhalation of a disclosed composition.

In another embodiment of the present disclosure, a method is provided, wherein the biological or chemical damage is caused by exposure to excess oxygen, any chemical that damages lung tissue, viral lung infection, bacterial lung infection, a genetic defect or a disease, vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS), either singly or a combination thereof.

In another embodiment of the present disclosure, a method is provided, wherein the chemical damage is a hyperoxic condition.

In another embodiment of the present disclosure, a method is provided in which the biological shock pathways include the wnt pathway, apoptotic responses, necrosis, initiation of fibrosis, or initiation of scarring.

In another embodiment of the present disclosure, a method is provided, in which proteins selected from Beta-catenin, Activin Receptor-like Kinase 5, and one or more proteins of the wnt pathway, either alone or in combination, are prevented from achieving concentration levels consistent with the initiation or continuation of biological processes leading to lung physiology malformation.

In another embodiment of the present disclosure, a method is provided, in which the treatment is applied to a human or an animal.

In another embodiment of the present disclosure, a method is provided, in which the treatment is applied to a prematurely born neonate.

In another embodiment of the present disclosure, a method of achieving healthy normal levels of peroxisome proliferator-activated receptor gamma in lung tissue is provided, comprising administration by inhalation of a disclosed composition.

In another embodiment of the present disclosure, a method of achieving healthy normal levels of activin receptor-like kinase 5 in lung tissue is provided, comprising administration by inhalation of a disclosed composition.

In another embodiment of the present disclosure, a method of achieving healthy normal levels of surfactant protein C is provided, comprising administration by inhalation of a disclosed composition.

In another embodiment of the present disclosure, a method of in preventing β-catenin from reaching concentration levels consistent with the initiation or continuation of biological processes leading to lung physiology malformation, comprising administration by inhalation of a disclosed composition.

In another embodiment of the present disclosure, a method of achieving healthy normal development of lung tissue physiology is provided, comprising administration by inhalation of a pharmaceutical composition.

In another embodiment of the present disclosure, a method of achieving healthy normal alveolar morphology is provided, comprising administration by inhalation of a disclosed composition.

In another embodiment of the present disclosure, a method of achieving healthy normal alveolar count is provided, comprising administration by inhalation of a disclosed composition.

In another embodiment of the present disclosure, a method of achieving healthy normal alveolar size is provided, comprising administration by inhalation of a disclosed composition.

In another embodiment of the present disclosure, a method of achieving healthy normal alveolar septal thickness is provided, comprising administration by inhalation of a disclosed composition.

In another embodiment of the present disclosure, a pharmaceutical composition is provided, for use of achieving healthy normal levels of peroxisome proliferator-activated receptor gamma in lung tissue.

In another embodiment of the present disclosure, a pharmaceutical composition is provided, for use of achieving healthy normal levels of activin receptor-like kinase 5 in lung tissue.

In another embodiment of the present disclosure, a pharmaceutical composition is provided, for use of achieving healthy normal levels of surfactant protein C.

In another embodiment of the present disclosure, a pharmaceutical composition is provided, for use in preventing β-catenin from reaching concentration levels consistent with the initiation or continuation of biological processes leading to lung physiology malformation.

In another embodiment of the present disclosure, a pharmaceutical composition is provided, for use of achieving healthy normal levels of lung tissue physiology.

In another embodiment of the present disclosure, a pharmaceutical composition is provided, for use of achieving healthy normal alveolar morphology.

In another embodiment of the present disclosure, a pharmaceutical composition is provided, for use of achieving healthy normal alveolar count.

In another embodiment of the present disclosure, a pharmaceutical composition is provided, for use of achieving healthy normal alveolar size.

In another embodiment of the present disclosure, a pharmaceutical composition is provided, for use of achieving healthy normal alveolar septal thickness.

In another embodiment of the present disclosure, a pharmaceutical composition is provided for use in achieving or maintaining healthy normal lung maturation.

In another embodiment of the present disclosure, a pharmaceutical composition is provided for use in achieving or maintaining healthy normal lung maturation wherein the healthy normal lung maturation status comprises one or more favorable characteristics of lung tissue histology or levels of one or more indicators of metabolic status, or a combination thereof.

In another embodiment of the present disclosure, a pharmaceutical composition is provided for use in achieving or maintaining healthy normal lung maturation wherein the lung histology characteristic comprises maintenance of healthy levels of alveolar count, alveolar size, alveolar septal thickness, or alveolar neutrophil infiltration or interstitial neutrophil infiltration, or a combination thereof.

In another embodiment of the present disclosure, a pharmaceutical composition is provided use in achieving or maintaining healthy normal lung maturation where the metabolic status comprises maintenance of healthy levels of Surfactant Protein A, Surfactant Protein B, Surfactant Protein C, Surfactant Protein D, Peroxisome

Proliferator-activated Receptor Gamma, BCL-2 Protein, BCL-2 Associated X Protein, Retinoic Acid X Receptor Alpha, Retinoic Acid X Receptor Beta, Retinoic Acid X Receptor Gamma, Vascular Endothelial Growth Factor, T-complex Protein 1 Subunit Alpha (also known as CTP:Phosphocholine Cytidylyltransferase Subunit Alpha), Fetal Liver Kinase 1, Beta-catenin, Activin Receptor-Like Kinase 5, or lung surfactant phospholipid synthesis rate, either singly or in a combination thereof.

In another embodiment of the present disclosure, a pharmaceutical composition is provided for use in administration by inhalation for avoiding damage to normal healthy lung physiology or initiation of biological shock pathways leading to malformation of lung in any manner differing from healthy normal lung physiology that may be initiated by biological or chemical damage to lung tissue.

In another embodiment of the present disclosure, a pharmaceutical composition for use is provided, wherein the biological or chemical damage is caused by exposure to excess oxygen, any chemical that damages lung tissue, viral lung infection, bacterial lung infection, a genetic defect or a disease, vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS), either singly or a combination thereof.

In another embodiment of the present disclosure, a pharmaceutical composition is provided for use is provided, wherein the chemical damage is a hyperoxic condition, vitamin deficiency disorders (including but not limited to nutritional insufficiency, premature birth, genetic defect, or induced pursuant to infection or disease states), hyperoxia damage, oxidative lung injury (lung damage), lung damage caused by chemical or environmental exposure, lung damage caused by a biological/infectious agent, including but not limited to bacterial infections and/or viral infections, lung damage caused by a chemical, biological, or radio-nuclear (CBRN) agent, and lung damage caused by radio-nuclear exposure, including but not limited to acute radiation-induced lung injury/damage and/or chronic lung injury/damage, including but not limited to delayed effects of acute radiations exposure (DEARS).

In another embodiment of the present disclosure, a pharmaceutical composition for use is provided in which the biological shock pathways include the wnt pathway, apoptotic responses, necrosis, initiation of fibrosis, or initiation of scarring.

In another embodiment of the present disclosure, a pharmaceutical composition is provided in which proteins selected from Beta-catenin, Activin Receptor-like Kinase 5, and one or more proteins of the wnt pathway, either alone or in combination, are prevented from achieving concentration levels consistent with the initiation or continuation of biological processes leading to lung physiology malformation.

In another embodiment of the present disclosure, a pharmaceutical composition for the use is provided for treating a human or an animal.

In another embodiment of the present disclosure, a pharmaceutical composition for use is provided for treating a prematurely born neonate.

In another embodiment of the present disclosure, a pharmaceutical composition suitable for dry powder inhalation is provided, comprising a therapeutically effective amount of vitamin A, or a pharmaceutically acceptable isomer, analog, and/or derivative thereof, or a mixture thereof, and a pharmaceutically acceptable carrier.

In another embodiment of the present disclosure, a pharmaceutical composition is provided, comprising all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, CD437 or CD1530, or a mixture thereof, or the palmitate, acetate, or maleate ester of all trans retinol, 13-cis-retinol, 9-cis-retinol, 9,13-dicis-retinol, 3,4-didehydroretinol, CD437, or CD-1530 or the mixture thereof, comprising all trans retinol, 13-cis-retinol, 9-cis-retinol, 9,13-dicis-retinol, 3,4-didehydroretinol, or the palmitate, acetate, or maleate ester of all trans retinol, 13-cis-retinol, 9-cis-retinol, 9,13-dicis-retinol, 3,4-didehydroretinol, or a mixture thereof, and a pharmaceutically acceptable carrier.

In another embodiment of the present disclosure, a pharmaceutical composition is provided, comprising a therapeutically effective amount of all trans retinol, or the palmitate, acetate, or maleate ester of all trans retinol, and a pharmaceutically acceptable carrier.

In another embodiment of the present composition, a pharmaceutical composition is provided, comprising a therapeutically effective amount of vitamin A, or an isomer, analog, and/or derivative thereof, or a mixture thereof, and a pharmaceutically acceptable carrier.

In another embodiment of the present disclosure, a pharmaceutical composition is provided, further comprising, Vitamin B, Vitamin C, Vitamin D, Vitamin D, Vitamin E, Vitamin K, or an analog and/or derivative of Vitamin B, Vitamin C, Vitamin D, Vitamin E, Vitamin K, or a mixture thereof.

In another embodiment of the present disclosure, a pharmaceutical composition is provided, further comprising one or more antibiotic compounds, or a mixture thereof.

In another embodiment of the present disclosure, a pharmaceutical composition is provided, wherein the antibiotic compound or compounds is selected from penicillins, tetracyclines, cephalosporins, quinolones, lincomycins, macrolides, sulfonamides, glycopeptides, aminoglycosides and carbapenems, and mixtures thereof.

In another embodiment of the present disclosure, a pharmaceutical composition is provided, further comprising an anti-viral compound.

In another embodiment of the present disclosure, a pharmaceutical composition is provided, wherein the anti-viral compound is selected from oseltamivir, zanamivir, peramivir, ribavirin, remdesivir, an nucleoside analog or analogs, an interferon or interferons, a protease inhibitor or inhibitors, a reverse transcriptase inhibitor, and a neuramidinidase inhibitor or inhibitors, or a mixture thereof.

In another embodiment of the present disclosure, a pharmaceutical composition is provided, wherein the pharmaceutically acceptable carrier comprises a sugar or mixture of sugars.

In another embodiment of the present disclosure, a pharmaceutical composition is provided, wherein the pharmaceutically acceptable carrier comprises a sugar or mixture of sugars.

In another embodiment of the present disclosure, a pharmaceutical composition is provided, wherein the sugar is selected from glucose, arabinose, maltose, sucrose, dextrose and lactose, and a mixture thereof.

In another embodiment of the present disclosure, a method for the treatment or prophylaxis of an infection caused by one or more infectious agents is provided, comprising administration to a patient in need thereof of a therapeutically effective amount of a pharmaceutical composition.

In another embodiment of the present disclosure, a method is provided, wherein the administration is by inhalation.

In another embodiment of the present disclosure, a method is provided, wherein the administration is by intravenous or intraarterial parenteral administration.

In another embodiment of the present disclosure, a method is provided, wherein an administration of a disclosed composition is by oral administration.

In another embodiment of the present disclosure, a method is provided, wherein the infectious agent is one or more bacterial agents or one or more viral agents, or one or more mycobacterial agents, or a combination thereof.

In another embodiment of the present disclosure, a method is provided, wherein the infectious agent is one or more antibiotic resistant bacteria.

In another embodiment of the present disclosure, a method is provided, wherein the infectious agent is methicillin resistant Staphylococcus aureus.

In another embodiment of the present disclosure, a method is provided, wherein the viral agent is a coronavirus.

In another embodiment of the present disclosure, a method is provided, wherein the coronavirus is SARS-CoV-2 and related viridae.

In another embodiment of the present disclosure, a method is provided, wherein the infectious agent or agents target the respiratory pathway as the primary location of entry.

In another embodiment of the present disclosure, a method is provided, wherein the target of the respiratory pathway is the lung, the nasal passages, the oral cavity, or a combination thereof.

In another embodiment of the present disclosure, a method is provided, for minimizing lung damage caused by the biological/infectious agent or agents.

In another embodiment of the present disclosure, a method is provided, wherein the minimizing lung damage minimizes the onset of acute respiratory distress syndrome.

In another embodiment of the present disclosure, a method is provided, for the treatment or prophylaxis of a condition or conditions facilitated by the presence of the biological/infectious agent or agents.

In another embodiment of the present disclosure, a method is provided, wherein the condition being treated or prevented is iatrogenic damage to lung tissue caused by the introduction of increased oxygen levels used to counter reduced pulmonary function caused by a biological/infectious agent or agents.

In another embodiment of the present disclosure, a method is provided, wherein the treatment or prophylaxis achieves healthy normal alveolar morphology in a patient that has been treated for a biological/infectious agent or agents.

In another embodiment of the present disclosure, a method provided, wherein the treatment or prophylaxis achieves healthy normal alveolar septal thickness, radial alveolar count, or alveolar septal thickness, alone or in combination.

In another embodiment of the present disclosure, a method is provided, wherein the condition is selected from pneumonia, pneumonitis, other infections, asthma, angina, exacerbated arthritis, allergies, and intercalated infections, and a combination thereof.

In another embodiment of the present disclosure, a method of suppressing the over-stimulation of the immune response, in a patient in need thereof, is provided, comprising administration of a therapeutically effective amount of a composition.

In another embodiment of the present disclosure, a method of treating infection-induced vitamin A deficiency, in a patient in need thereof, is provided, comprising administration of a therapeutic effective amount of a disclosed composition.

In another embodiment of the present disclosure, a method of supporting a patient’s immune response is provided, comprising administration of a disclosed composition.

In another embodiment of the present disclosure, a method is provided, wherein the supporting comprises stimulation of phagosome maturation, or recruiting of white blood cells to the site of an infection.

In another embodiment of the present disclosure, a method of improving the efficacy of a vaccine against an infectious agent in a patient is provided, comprising administration of a disclosed composition.

In another embodiment of the present disclosure. a method of countering suppressed levels of bloodborne vitamin A induced by systemic response to an infectious agent in a patient is provided, comprising administration by inhalation of a disclosed composition.

In another embodiment of the present disclosure, a pharmaceutical composition is provided, for use in the treatment or prophylaxis of an infection caused by one or more infectious agents.

In another embodiment of the present disclosure, a pharmaceutical composition is provided, for use in the treatment or prophylaxis of an infection caused by one or more bacterial agents or one or more viral agents, or one or more mycobacterial agents, or a combination thereof.

In another embodiment of the present disclosure, a pharmaceutical composition is provided, for use in the treatment or prophylaxis of an infection caused by one or more antibiotic resistant bacteria.

In another embodiment of the present disclosure, a pharmaceutical composition is provided, for use in the treatment or prophylaxis of an infection caused by a methicillin resistant Staphyloccus aureus.

In another embodiment of the present disclosure, a pharmaceutical composition is provided, for use in the treatment or prophylaxis of an infection caused by a coronavirus, an influenza virus, or related viridae.

In another embodiment of the present disclosure, a pharmaceutical composition is provided, for use in the treatment or prophylaxis of an infection caused by SARS-CoV-2.

In another embodiment of the present disclosure, a pharmaceutical composition is provided, for use in the treatment or prophylaxis of an infection or infections that target the respiratory system as the primary location of entry.

In another embodiment of the present disclosure, a pharmaceutical composition is provided, for use in the treatment or prophylaxis of an infection or infections that target the respiratory system as the primary location of entry.

In another embodiment of the present disclosure, a pharmaceutical composition is provided, for use in the treatment or prophylaxis of an infection or infections that target the lung, the nasal pathway, the oral cavity, or a combination thereof.

In another embodiment of the present disclosure, a pharmaceutical composition is provided, for use in minimizing lung damage caused by an infectious agent or agents.

In another embodiment of the present disclosure, a pharmaceutical composition is provided, for use in minimizing the onset of acute respiratory distress syndrome.

In another embodiment of the present disclosure, a pharmaceutical composition is provided, for use in the treatment of prophylaxis of a condition or conditions facilitated by the presence of an infectious agent or agents.

In another embodiment of the present disclosure, a pharmaceutical composition is provided, for use in the treatment or prophylaxis of iatrogenic damage to lung tissue caused by the introduction of increased oxygen levels used to counter reduced pulmonary function caused by an infectious agent or agents.

In another embodiment of the present disclosure, a pharmaceutical composition is provided, for use of achieving healthy normal alveolar morphology in a patient that has been treated for an infectious agent or agents.

In another embodiment of the present disclosure, a pharmaceutical composition is provided, for use in achieving normal alveolar septal thickness, radial alveolar count, or alveolar septal thickness, alone or in combination.

In another embodiment of the present disclosure, a pharmaceutical composition is provided, for use in the treatment of prophylaxis of pneumonia, pneumonitis, other infections, asthma, angina, exacerbated arthritis, allergies, and intercalated infections, or a combination thereof.

In another embodiment of the present disclosure. a pharmaceutical composition is provided, for use in the suppression of over-stimulation of the immune response, in a patient in need thereof.

In another embodiment of the present disclosure, a pharmaceutical composition is provided, for use in the treatment of infection-induced vitamin A deficiency.

In another embodiment of the present disclosure, a pharmaceutical composition is provided, for use in supporting a patient’s immune response.

In another embodiment of the present disclosure a pharmaceutical composition, is provided, for use in the stimulation of phagosome maturation, or recruiting of white blood cells to the site of an infection.

In another embodiment of the present disclosure, a pharmaceutical composition is provided, for use in improving the efficacy of a vaccine against an infectious agent in a patient.

In another embodiment of the present disclosure, a pharmaceutical composition is provided, for use in countering suppressed levels of bloodborne vitamin A induced by systemic response to an infectious agent.

It is understood the disclosures herein may be combined. By way of a non-limiting example, disclosed is: (i) a pharmaceutical composition comprising between 2.5% and 3.0% vitamin A, or an isomer, analog, and/or derivative thereof, or a mixture thereof; and (ii) a method wherein an administration of a disclosed composition is by oral administration. Therefore, disclosed is a method wherein an administration of a disclosed composition is by oral administration and the disclosed composition comprises between 2.5% and 3.0% vitamin A, or an isomer, analog, and/or derivative thereof, or a mixture thereof.

### EXAMPLES

The following examples serve to more fully describe the manner of using the above-described disclosure, as well as to set forth the best mode contemplated for carrying out various aspects of the disclosure. The Examples according to the present disclosure are those falling within the scope of the claims herein.

It is noted that examples in this disclosure involve hyperoxia as a mode of lung damage in model animals. Hyperoxia is a damage mode applicable to humans or animals regardless of age. Hyperoxia, as detailed in this disclosure, is also used with newborn rats as a model of human BPD. As such, results presented herein for treatment or prophylaxis, based on hyperoxia damage to lung, directly address hyperoxia damage to lung of any age or developmental status, and the lung disfigurement associated with BPD.

### Example 1: Inhaled Vitamin A Doses Support Normal Lung Tissue Development and Formation of Healthy Lung Tissue Morphology, Preventing Lung Damage in a Model of BPD

Hyperoxia exposure, a well-accepted model of BPD (Dasgupta, C., Sakurai, R., et al, Hyperoxia-induced neonatal rat lung injury involves activation of TGF-beta and Wnt signaling and is protected by rosiglitazone. Am J Physiol Lung Cell Mol Physiol 296: L1031-1041, 2009), was used to study the effect of vitamin A treatment, using methods and techniques previously developed to study this BPD model system (Dasgupta et al 2009, Morales E., Sakurai R., et al, Nebulized PPARγ Agonists: A Novel Approach to Augment Neonatal Lung Maturation and Injury Repair. Pediatr Res 75: 631-640, 2014; Richter J., Toelen, J., et al, Functional assessment of hyperoxia-induced lung injury after preterm birth in the rabbit. Am J Physiol Lung Cell Mol Physiol 306: L277-283, 2014; Sakurai, R., Villareal, P., et al, Curcumin protects the developing lung against long-term hyperoxic injury. Am J Physiol Lung Cell Mol Physiol 305: L301-311, 2013). Lung tissue morphology and biomarkers were analyzed to observe the simultaneous but opposing effects of damaging hyperoxia upon lung maturation and lung tissue damage/repair. Lung maturation is ongoing in neonatal rats, normally born with lungs in the saccular state and still maturing during the first days after normal full-term birth, making an ideal and accessible model for mimicking the development status of the lungs of premature human neonates. Untreated hyperoxia of still-maturing neonatal lung is known to disrupt normal maturation, including being a root cause of eventual development of BPD. The effects of hyperoxia can be measured by monitoring disruption or alteration of the course of normal lung maturation, and also by the induction of damage repair or shock pathways. Exceeding mere equivalence to IM dosing, inhaled vitamin A dosing gave statistically an unexpectedly superior prophylactic performance across a number of metrics, supporting the advantages of the present disclosure of direct-to-target dosing, offering a BPD preventative measure, while simultaneously being a non-invasive dose route that overcomes concerns of repeated IM dosing in neonates.

### Materials and Methods

Animals and conditions: Time-mated pregnant Sprague-Dawley rat dams dwelled under normoxia through litter delivery. Per protocols previously described (see, for example, Morales E, Sakurai R, et al. Nebulized PPARγ Agonists: A Novel Approach to Augment Neonatal Lung Maturation and Injury Repair. Pediatr Res. 2014 May;75(5); PMCID: PMC4016987; Sakurai R, Villarreal P, et al., Curcumin protects the developing lung against long-term hyperoxic injury. Am J Physiol Lung Cell Mol Physiol. 201 Aug 15:305(4):L301-311. PMCID: PNC3891014; Dasgupta C, et al., Hyperoxia-induced neonatal rat lung injury involves activation of TGF-beta and Wnt signaling and is protected by rosiglitazone. Am J Physiol Lung Cell Mol Physiol. 2009 Jun; 296(6): L1031-1041. PMCID: PMC3286237; and Richter J, et al., Functional assessment of hyperoxia-induced lung injury after preterm birth in the rabbit. Am J Physiol Lung Cell Mol Physiol. 2014 Feb;306(3): L277-283. PMID: 24375793), pre-weaned neonatal rats and dams were segregated into hyperoxia or normoxia groups on the first day after litter delivery (postnatal day one (PD=1) and were maintained continuously in those condtions throughout the study. Pups were separated from dams only to receive the vitamin A or vehicle dosing. Throughout these studies, sex was tracked as a response variable, but no statistical differences were detected in any of our parameters.

Vitamin A formulation: A water-miscible vitamin A palmitate was prepared as a solution of 50,000 IU/ml, containing only pharmaceutical grade vitamin A palmitate (DSM Nutritionals, Parsippany, New Jersey) solubilized with 12% polysorbate 80, with the balance of the volume being water only, except, if necessary, adding small amounts of citric acid or sodium hydroxide to achieve neutral pH. A vehicle-only solution was also prepared, lacking only vitamin A palmitate but otherwise identical. Vitamin A and vehicle solutions were sterile-filtered through 0.22 micron membranes as a final step in preparation.

Vitamin A dose level: The target vitamin A dose for neonatal rats was 5 IU/g body mass, scaled by body mass based on typical NICU dosing dosing as follows: NICU dosing is 5,000 IU delivered IM every 48 hours, and 1 kg average body mass for an extremely low birth weight (ELBW) premature human baby. Animals were weighed to determine the exact dose at each timepoint. Dosing was performed on alternate days (PD=1, 3, 5 and 7), maintaining the 48 hour cadence of the typical NICU course (although, it is acknowledged that the development cycle of newborn rats is accelerated versus human neonates). The stock vitamin A formulation was diluted as necessary in sterile saline to facilitate accurate dose delivery by either injection or inhalation routes. Vehicle doses were of identical volume and formulation, only lacking vitamin A, and including the same saline dilution.

Inhalation dosing: Whole-body aerosol exposure was performed using previously described and standardized methods (see, for example, Morales E, Sakurai R, et al. Nebulized PPARγ Agonists: A Novel Approach to Augment Neonatal Lung Maturation and Injury Repair. Pediatr Res. 2014 May;75(5); PMCID: PMC4016987; and Taylor SK, et al., Inhaled Vitamin D″: A Novel Strategy to Enhance Neonatal Lung Maturation. Lung. 2016 Dec 1:194(6):931-43. PMCID: PMC5191914). A 5-fold excess of the target vitamin A dose was nebulized, to account for the assumption of approximately 20% dose delivery efficiency that has been described previously (O′Callaghan C & Barry PW. The science of nebulised drug delivery. Thorax 52 Suppl 2: S31-44, 1997; Rau JL. Design principles of liquid nebulization devices currently in use. Respir Care 47: 1257-1275; discussion 1275-1278, 2002). As such, 25 IU/g body mass was aerosolized for each animal. Pups were typically dosed communally in a single whole-body exposure chamber, maintaining, as appropriate, the normoxia or hyperoxia conditions during the aerosol exposure. The cup of a vibrating-mesh nebulizer (Aerogen®, Galway, Ireland) was loaded with a final volume of 1.5 ml, comprised of vitamin A or vehicle stocks diluted into sterile isotonic saline, then was aerosolized in the exposure chamber for inhalation. Pups (without dams) dwell in the chamber for 30 minutes to ensure thorough respiratory deposition, well beyond the time for complete nebulization of the 1.5 ml (approximately 12 minutes maximum).

Samples: Animals were euthanized on PD=7, several hours after any PD=7 exposure, to collect lungs and blood. Portions of lung were either snap-frozen, paraformaldehyde-inflation fixed, or cultured as explants, following previously described methods ((see, for example, Morales E, Sakurai R, et al. Nebulized PPARγ Agonists: A Novel Approach to Augment Neonatal Lung Maturation and Injury Repair. Pediatr Res. 2014 May;75(5); PMCID: PMC4016987; and Taylor SK, et al., Inhaled Vitamin D″: A Novel Strategy to Enhance Neonatal Lung Maturation. Lung. 2016 Dec 1:194(6):931-43. PMCID: PMC5191914)). Blood was allowed to clot and centrifuged to yield serum which was quickly aliquoted and frozen. All samples were stored at -80° C. and freeze-thaw cycling was minimized to preserve sample integrity.

Lung morphometry: For lung morphometry, radial alveolar count (RAC), mean linear intercept (MLI), and alveolar septal thickness (AST) were determined following previously described methods (Dasgupta C, Sakurai R, et al., Hyperoxia-induced neonatal rat lung injury involves activation of TGF-{beta} and Wnt signaling and is protected by rosiglitazone. Am J Physiol Lung Cell Mol Physiol 296: L1031-1041, 2009).

Protein extraction and western blot analysis: Liquid nitrogen flash-frozen lung tissue was homogenized with a tissue grinder in lysis buffer (RIPA buffer) containing 1 mM EDTA and EGTA each (Boston Bioproducts Ashland, MA), supplemented with 1 mM PMSF, and complete protease inhibitor cocktail (Roche Diagnostics, Indianapolis, IN). For each sample, 50 µg of total protein was denatured with SDS sample buffer and electrophoresed in 10% SDS polyacrylamide gel. Resolved samples were then transferred onto a 0.45-µm nitrocellulose membrane and, after blocking with TBS-Tween (TBST) + 5% milk, were probed with primary antibodies [Bcl-2 (1:200, cat# sc-492), Bcl-2-associated X protein (Bax; 1:350; cat# sc-493), peroxisome proliferator-activated receptor gamma (PPAR-γ; 1:500; cat # sc-7196), surfactant protein C (SP-C; 1:250; cat # sc-518029), CTP:phosphocholine cytidylyltransferase subunit alpha (CCT-α; 1:200; cat # sc-376107), β-catenin (1:500; cat# sc-7963), activin receptor-like kinase 5 (ALK-5; 1;200; cat# sc-398), fetal liver kinase 1 (FLK-1; 1:200; cat# sc-6251) (all from Santa Cruz Biotechnology, Santa Cruz, CA), glyceraldehyde 3-phosphate dehydrogenase (GAPDH; 1:5,000; cat# MAb374, MilliporeSigma, Burlington, MA) and retinoid X receptor alpha (RXRα; 1:800; cat# NBP2-20130, Novus Biologics, Littleton, CO)] overnight at 4° C., followed by appropriate secondary antibody and Super-Signal Chemiluminescent substrate (Pierce Chemicals, Rockford, IL). The validation and specificity of the antibodies used have been demonstrated in previous publications (Dasgupta C et al., “Hyperoxia-induced neonatal rat lung injury involves activation of TGF-beta and Wnt signaling and is protected by rosiglitazone”, Am J Physiol Lung Cell Mol Physiol 296: L1031-L1041, 2009; Kalmar GB et al., “Primary structure and expression of a human CTP:phosphocholine cytidylyltransferase”, Biochim Biophys Acta 1219: 328-334, 1994; Rehan VK et al., “Perinatal nicotine-induced transgenerational asthma”, Am J Physiol Lung Cell Mol Physiol 305: L501-L507, 2013; Sakurai R et al., “Perinatal nicotine exposure induces myogenic differentiation, but not epithelial-mesenchymal transition in rat offspring lung”, Pediatr Pulmonol 51: 1142-1150, 2016), specifically, demonstrating alveolar cell specific localization of SP-C, β-catenin, and PPARγ by immunostaining for the relevant cell-specific markers. ImageJ software (National Institutes of Health, Bethesda, MD) was used to quantitate protein bands, which were measured by densitometry and expressed relative to accompanying GAPDH bands as relative units.

Immunofluorescence: Immunofluorescence staining of PPAR-γ; 1:50, β-catenin; 1:100 and RXRα; 1:200 was performed as previously described (Morales E, Sakurai R, Husain S, Paek D, Gong M, Ibe B, Li Y, Husain M, Torday JS, Rehan VK. Nebulized PPARγ Agonists: A Novel Approach to Augment Neonatal Lung Maturation and Injury Repair. Pediatr Res 75: 631-640, 2014; Yurt M, Liu J, et al., Vitamin D supplementation blocks pulmonary structural and functional changes in a rat model of perinatal vitamin D deficiency. Am J Physiol Lung Cell Mol Physiol 307: L859-867, 2014.) and was merged using Photoshop software. Briefly, 5-µm sections were incubated with appropriate primary antibodies at 4° C. overnight; Alexa Fluor 594 donkey anti-mouse IgG (1:250 dilution for β-Catenin cat# A 10037, Invitrogen), and Alexa Flour 488 goat anti-rabbit IgG (1:250 dilution for RXRα, cat# A1137, Invitrogen), and Alexa Fluor 594 or 488 goat anti-rabbit IgG (1:250 dilution for PPARγ, cat# A11034 and A11037, Invitrogen) were applied to the sections for 15 min at room temperature. The sections were washed with phosphate-buffered saline and then mounted with Pro-Long Gold antifade reagent with DAPI (Invitrogen) for visualization under a fluorescence microscope by a single blinded investigator. Immunostaining for myeloperoxidase (MPO) [1:50, primary antibody (Cat: 22225-1-AP, Proteintech) and 1: 200 secondary antibody (anti rabbit, Cat: UC282766, Invitrogen)] was performed using previously described method (Dasgupta C, Sakurai R, et al, Hyperoxia-induced neonatal rat lung injury involves activation of TGF-{beta} and Wnt signaling and is protected by rosiglitazone. Am J Physiol Lung Cell Mol Physiol 296: L1031-1041, 2009).

Measurement of rate of surfactant phospholipid synthesis: De novo surfactant phospholipid synthesis, determined by the incorporation of [3H]choline chloride (NEN Dupont, Boston, MA) into DSPC, in cultured lung explants, as described previously (Rehan VK, Wang Y, et al., In utero nicotine exposure alters fetal rat lung alveolar type II cell proliferation, differentiation, and metabolism. Am J Physiol Lung Cell Mol Physiol 292: L323-333, 2007).

Enzyme linked immunosorbent assay: ELISA was performed following manufacturer’s protocol (Aviva Systems Biology, San Diego, CA).

For all analyses, the study design included paired animal groups to enable evaluation of vitamin A dosing versus vehicle dosing, as well as comparison of inhalation-dosing versus intramuscular (IM) dosing. Sex was also tracked as a variable, although there was no evidence of statistically significant sex bias in the data.

### Results

### Example 1a

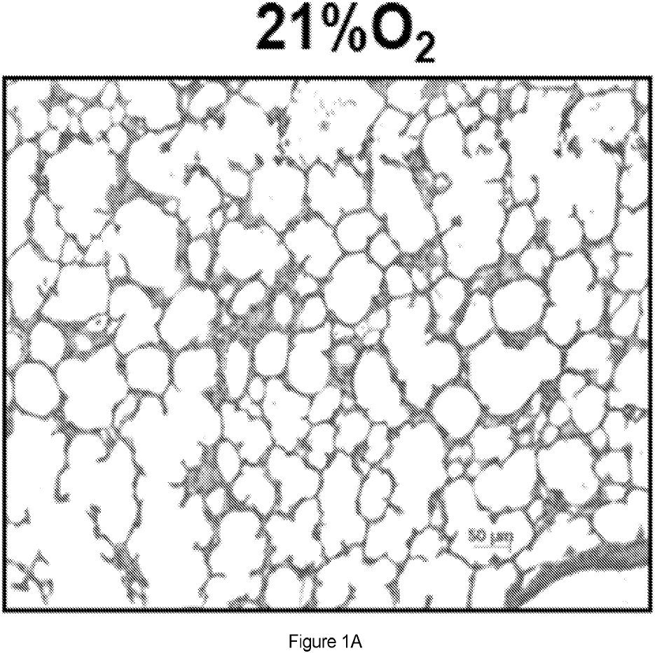

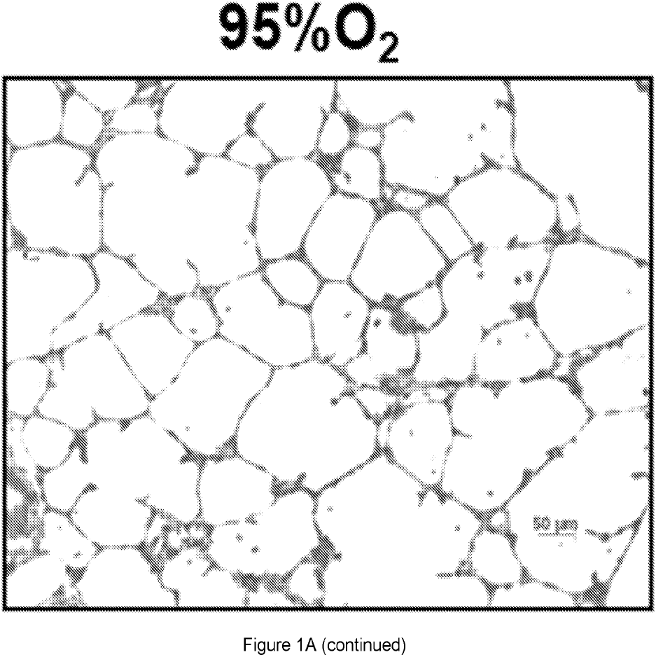

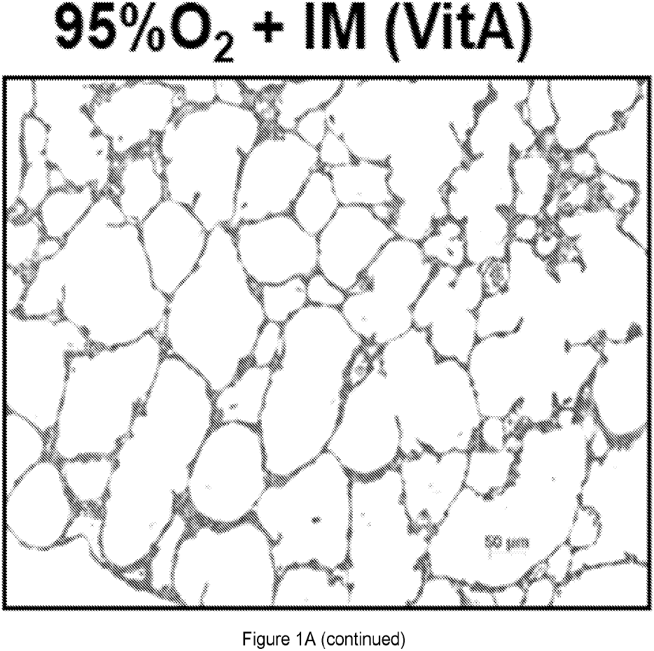

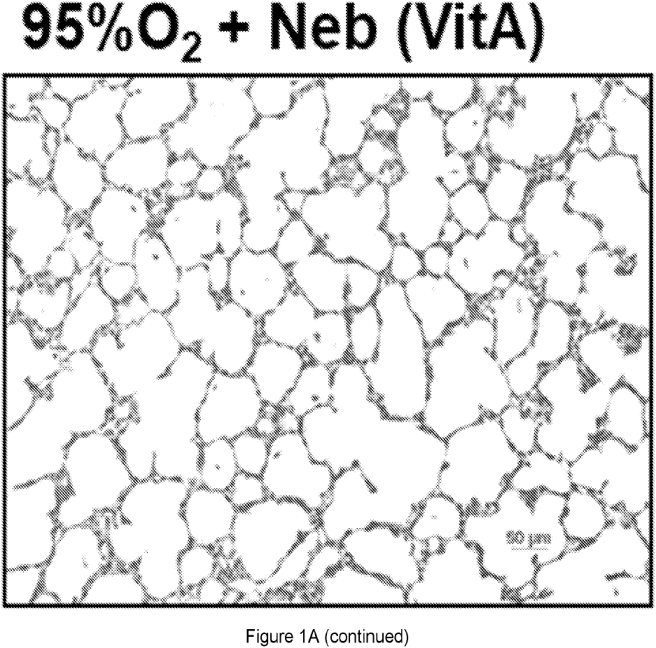

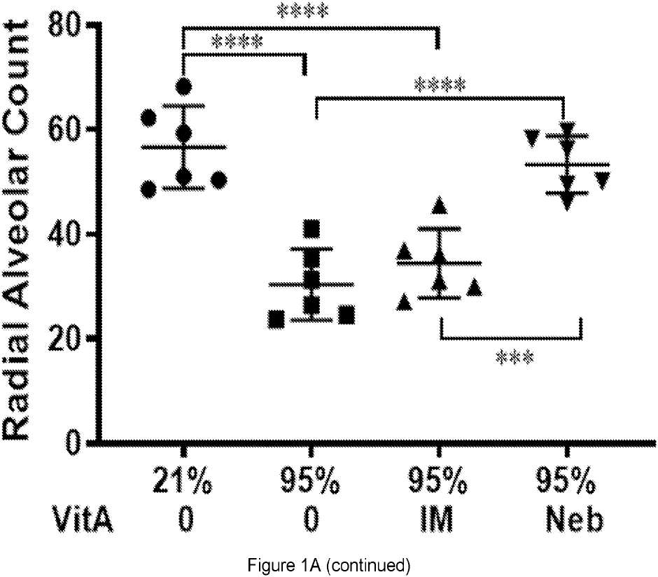

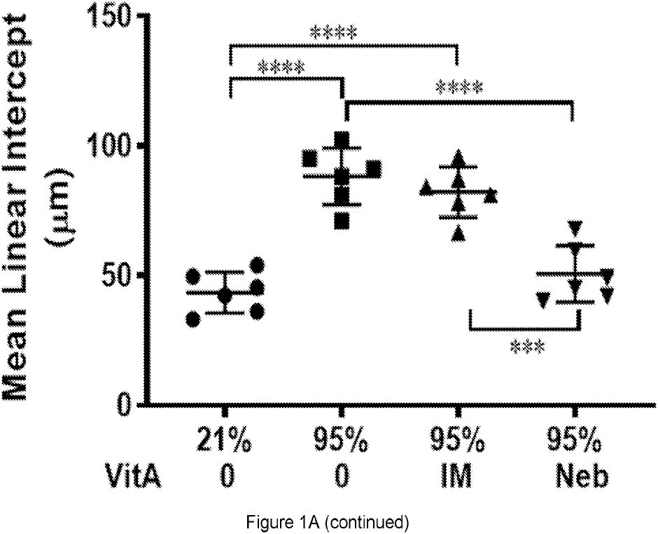

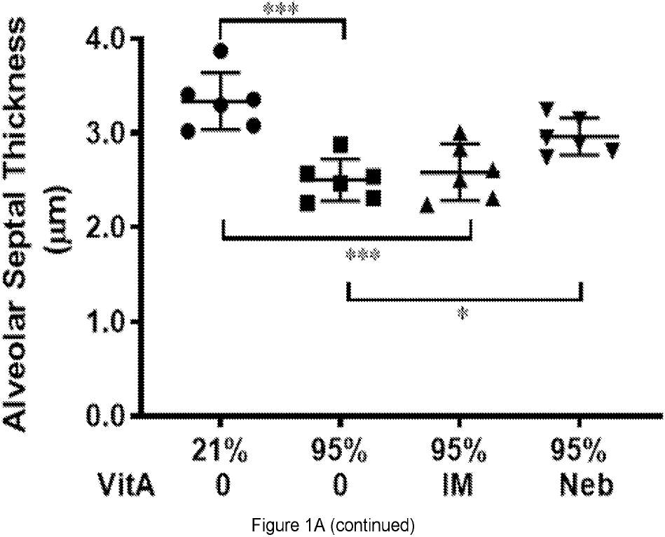

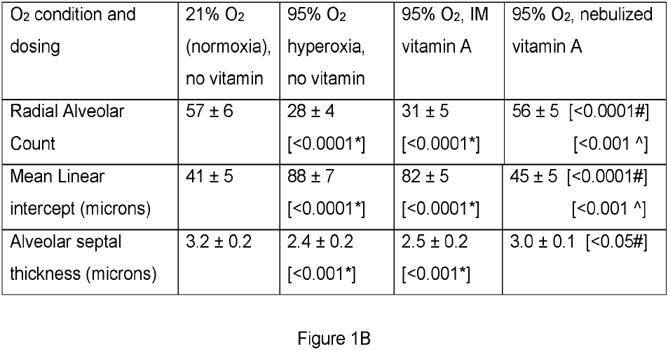

Lung morphology (FIGS. 1A and 1B) was judged by evaluating stained sections for radial alveolar count (RAC), mean linear intercept (MLI), and alveolar septal thickness (AST).

The expected damage of otherwise untreated (vehicle-treated) hyperoxia is easily evident (2^{nd} from left image) versus normoxia control lung (left image). These respective conditions also define the boundaries of metrics reflecting lung tissue morphology under otherwise untreated hyperoxia damage and the ideal healthy normal control state, setting the range within which the effect of vitamin A treatment of hyperoxia may be examined. IM vitamin A treatment (2^{nd} from right image) results in a modest change away from untreated hyperoxia, but with at best only marginal statistical discrimination. By contrast, inhaled vitamin A (right image) unexpectedly induced a noticeably more vigorous response, resulting in nearly healthy RAC and MLI, while AST is approximately only half-way to healthy but also far more potently restored toward the healthy condition versus IM dosing. This result was unexpected because, before the present disclosure and assuming arguendo that inhaled vitamin A formulations were even envisaged, inhaled vitamin A was envisioned to yield similar or perhaps slightly enhanced response compared to IM vitamin doses. Far from providing merely a slightly enhanced response, the dramatically superior lungs of the inhalation-dosed animals demonstrate the unexpected benefit of delivering vitamin A directly to the site of damage, such that the vast majority of the hyperoxia damage appears to be absent.

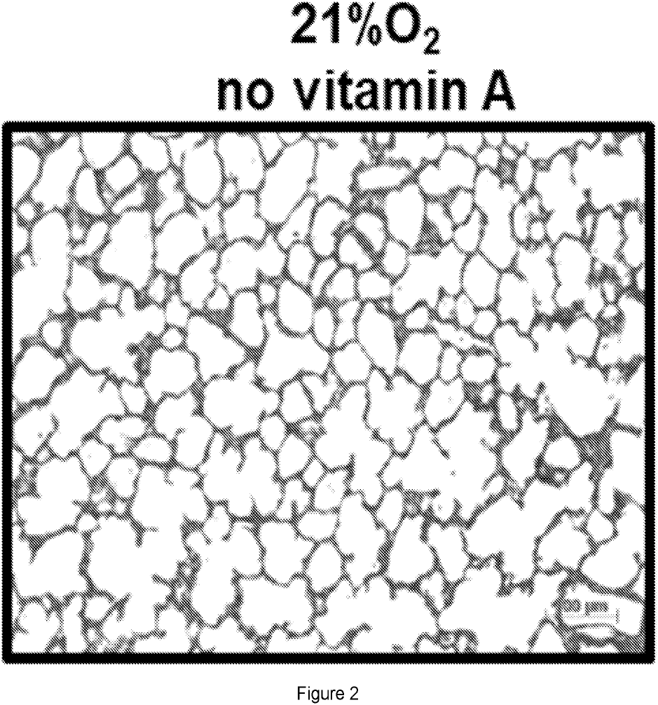

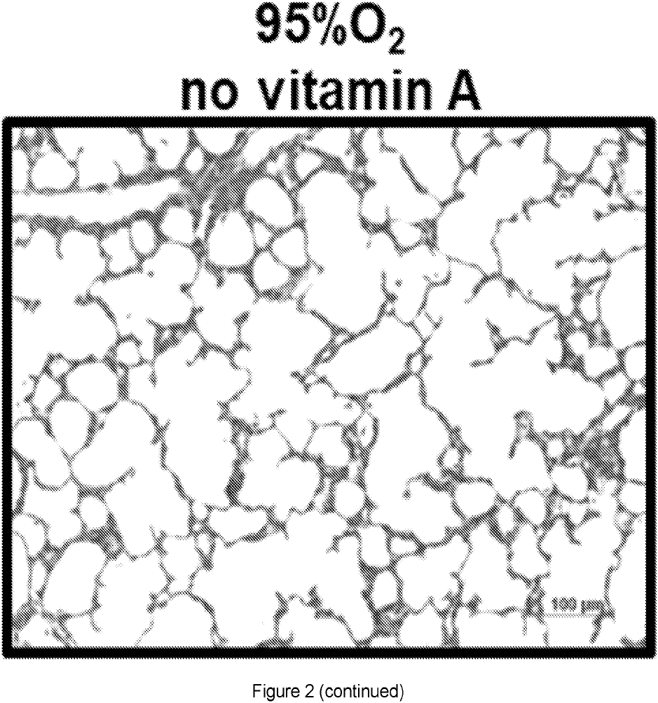

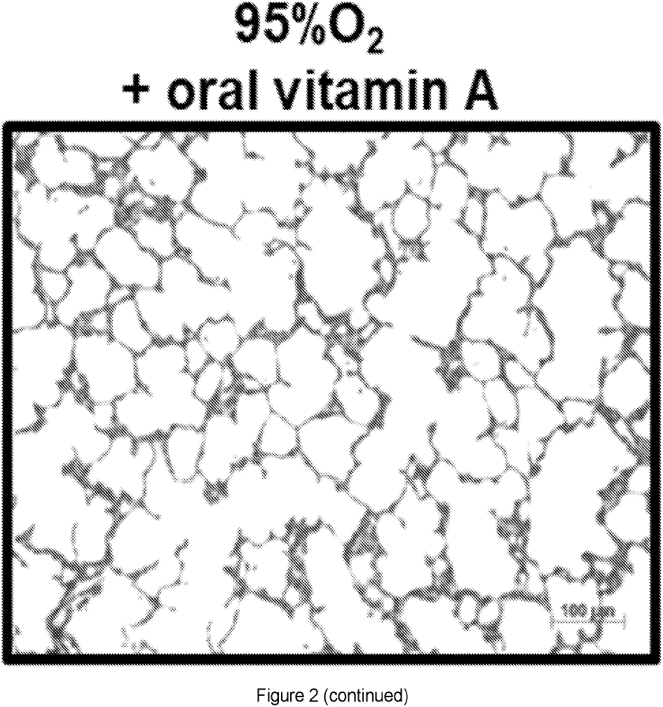

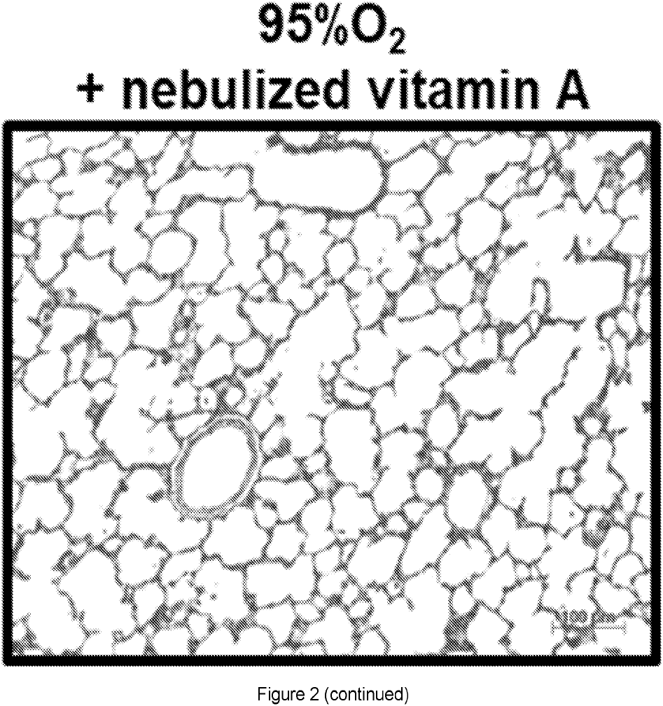

Similar testing reveals that oral dosing of vitamin A provides a similarly weak result as IM dosing for lung morphology under hyperoxia conditions (FIG. 2) when compared to the unexpectedly potent outcome seen with inhaled vitamin A dosing. In FIG. 2, the oral doses, delivered by intra-esophageal gavage instead of intramuscular injection, are the same level and frequency as IM dosing (as in FIGS. 1A and 1B). FIG. 2 shows representative lung morphology images from single animals under the oxygen and treatment conditions described. The results are visually clear in these images: oral vitamin A doses result in lung morphology similar to untreated hyperoxia damage, while inhaled nebulized vitamin A doses result in lung very similar to normal healthy lung.

It is important to consider the ranges of conditions that can lead to hyperoxia exposure, defined as exposure to oxygen at higher levels than in the normal breathable atmosphere. In the womb, developing lung tissue is never exposed to air; as such, premature birth babies are exposed to hyperoxia merely by their lungs being exposed to normal air (Tin, W, and S Gupta. “Optimum oxygen therapy in preterm babies.” Archives of disease in childhood. Fetal and neonatal edition vol. 92,2 (2007): F143-7. doi:10.1136/adc.2005.092726; Campbell, K. (1951) “Intensive oxygen therapy as a possible cause of retrolental fibroplasia; a clinical approach.” The Medical Journal of Australia, 2(2), 48.). Use of severe hyperoxia exposure as the animal model in these examples is meant to model human neonatal bronchopulmonary dysplasia (BPD). The disclosed model also serves more generally as a model for any human patient needing to be provided with increased oxygen as a life-saving measure to treat any cause of acute pulmonary insufficiency.

The damage caused by oxygen, without other intervention, is clear, with distinct loss of small alveoli, a feature of healthy lung which are critical for normal respiratory capacity. The example shown represents evidence of unexpected efficacy of inhaled vitamin A for mitigating the extent of hyperoxia damage to lung tissue, even under the exquisitely harsh hyperoxia condition used in the experiment. Injected vitamin A, even though at the same dose (and likely a higher effectively delivered dose because the injection yields 100% delivery of the intended dose), produces very little benefit to the lung. The advantage and unique potency of the inhaled vitamin A dose is clearly demonstrated versus the far weaker responses induced by the injected dose.

### Example 1b: Stimulation of Lung Maturation by Inhaled Vitamin A Is Also Apparent at the Biochemical Level

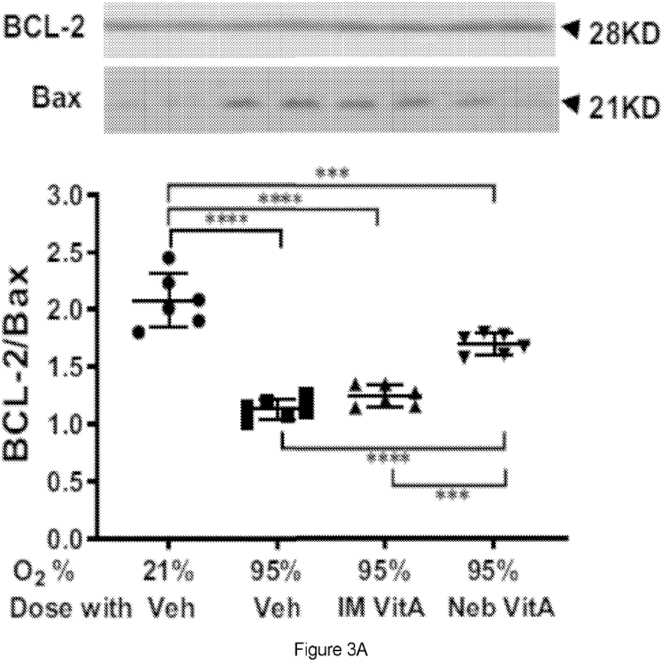

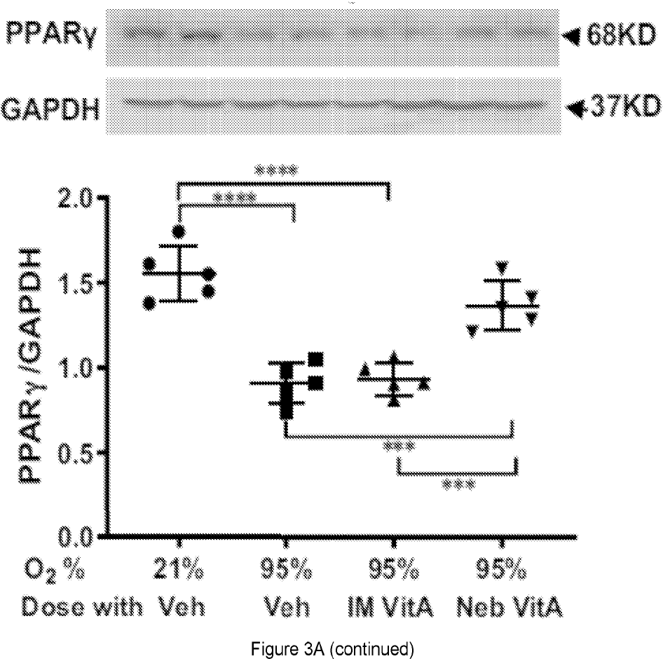

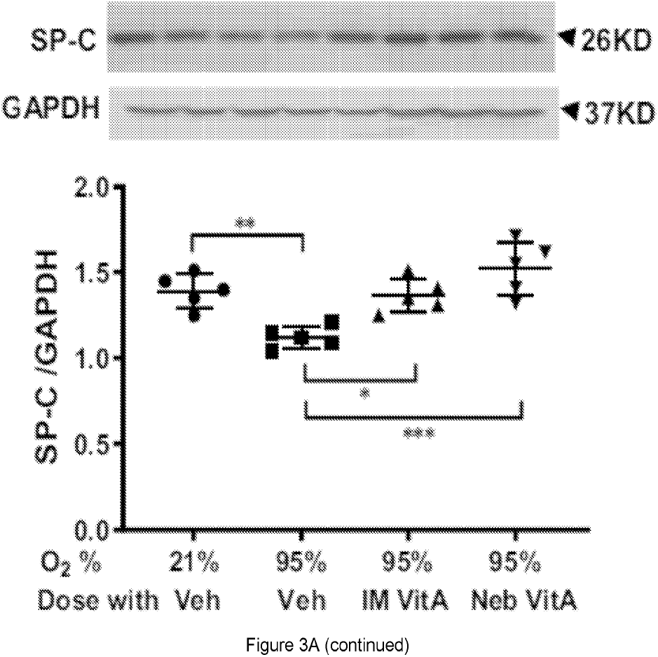

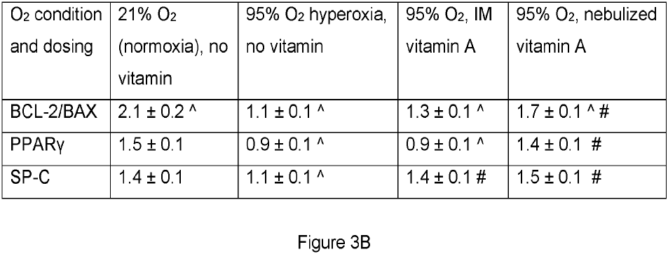

Biomarkers of lung maturation respond to hyperoxia and vitamin A dosing in a largely parallel manner as the lung morphology response. Lung maturation is demonstrably disrupted by upon untreated hyperoxia and vitamin A treatment reverses these effects (FIGS. 3A and 3B), with the effect being significantly stronger when the vitamin A is inhaled versus injected.

As with morphology, potent responses from inhaled vitamin A are evident in all cases, many achieving nearly normal-healthy levels. PPARγ (peroxisome proliferator-activated receptor gamma) is a protein and a member of the type II nuclear receptor family of proteins that regulate gene function. During embryonic tissue development, PPARγ function is critical for differentiation of tissues. A drop in PPARy levels induced by hyperoxia exposure of the newborn rats indicates that the oxygen-induced damage interferes with the biochemical processes of healthy tissue growth and differentiation. Likewise, the BCL-2/BAX ratio reflects normal growth and differentiation of cells (high levels of this ratio) versus an indication of a switch toward damaging apoptotic (programmed cell death) processes (lower levels of this ratio) - and with the same trends: untreated oxygen damage induces a shift toward destructive biochemical processes, whereas the inhaled vitamin A palmitate unexpectedly maintains the BCL-2/BAX ratio in the healthy range of normal tissue development. By contrast, injected vitamin A doses elicit only minimal if any benefits in countering the oxygen-induced damage to lung tissue maturation at the biochemical level.

Surfactant Protein C (SP-C) is one of the four common proteins in pulmonary surfactant, which is a complex mixture of proteins, lipids and phospholipids that is synthesized in lung cells and secreted into the lung lumen, the air-facing side of lung tissue. Pulmonary surfactant has several key biological functions, primarily in maintaining a proper surface tension that keeps air exposure from rupturing cells (the surface tension of exposing cell membranes to air causes the cells to burst) while also serving as a critical conduit through which oxygen and carbon dioxide can diffuse easily between the respired air and cells lining the alveoli. Surfactant proteins play a critical role in these functions, so a drop in their level upon untreated hyperoxia indicates impairment of pulmonary function, and particularly in these neonatal animals that lung maturation has been disrupted. Vitamin A doses allow maintenance of normal SP-C levels; in this case, the inhaled doses are as effective as the injected doses.

Taken as a whole, the inhaled vitamin A palmitate doses unexpectedly support lung maturation status across the variety of lung maturation metabolic processes that we examined, whereas injected doing shows little benefit except for pulmonary surfactant protein synthesis.

### Example 1c: Suppression by Inhaled Vitamin A of Adverse Metabolic Processes Initiated by Oxygen Damage to Lung Tissue

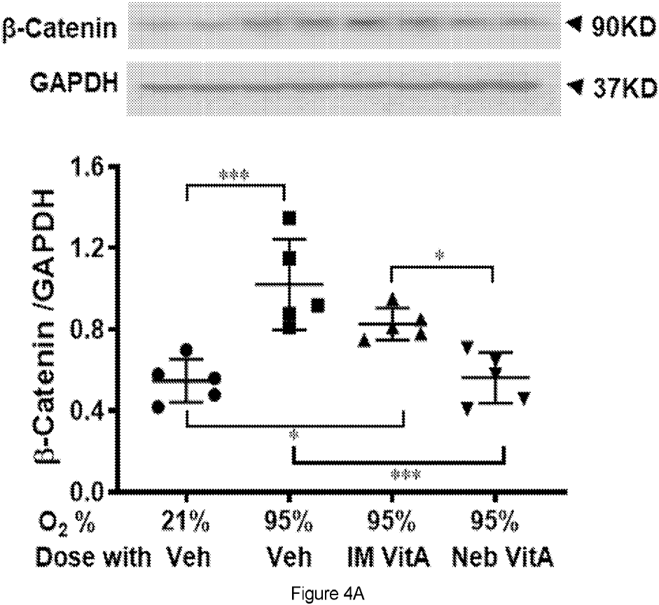

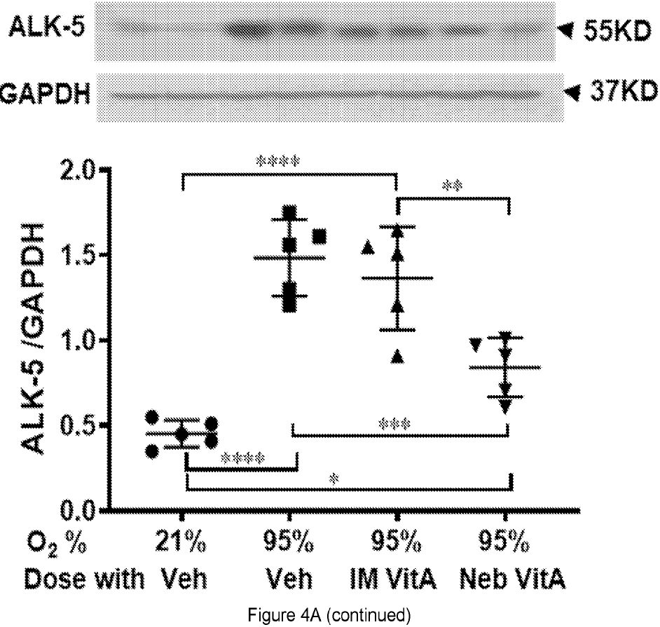

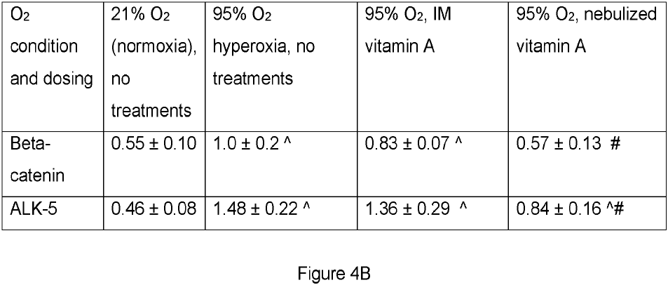

Equally important with inhaled vitamin A doses supporting tissue maturation is the opposite concern of avoiding damaging biochemical processes. For example, the Wnt pathway is a central biochemical pathway initiated in response to a variety of tissue damage — such pathways are critical to healing. However, if stimulated in excess, e.g. as a shock response to harsh damage, wnt and related pathways can lead to lasting tissue damage, which in the lung, can include initiation fibrous formations that disrupt normal lung features like small, functional alveoli, leading to ‘scarring.’ In premature babies and adults alike, the consequences of this outcome after hyperoxia exposure (including therapeutic hyperoxia) include life-long pulmonary dysfunction, sometimes severe and/or requiring ongoing medical care. The proteins β-catenin and activin receptor-like kinase 5 (ALK-5) (FIGS. 4A and 4B) are biomarkers indicative of wnt pathway activation, and more generally indicate activation of damage repair responses. In these markers, untreated oxygen damage elevates levels of these biomarkers, as would be expected. Again, the injected vitamin A shows essentially no suppression of these damage-repair pathways. Vitamin A inhalation, however, unexpectedly results in a marked decrease in these markers. Importantly, the response to inhaled vitamin A does not necessarily push the levels of these biomarkers all the way back to healthy normal levels (for example, ALK-5 in FIGS. 4A and 4B) — this is a critical and unexpected attribute, in that some level of damage-repair metabolic response is needed, since in all cases there will be oxygen-induced tissue damage that needs to be repaired. As such, driving the levels back to the healthy normal level would indicate in inappropriate cessation of repair. The intermediate biomarker levels upon inhalation dosing suggest induction of an appropriate level of beneficial repair without the unchecked ‘shock-like’ repair which can ultimately disfigure the lung.

It is also noted that the BCL-2/BAX ratio (FIGS. 3A and 3B) is also a reflection of avoiding overly damaging biological responses. Described above, maintaining a high ratio of BCL-2:BAX is an indication of supporting appropriate tissue maturation; but driving a low ratio would indicate activation of apoptosis (programmed cell death), with cells dying to forestall propagation of tissue damage. Apoptosis being avoided in favor of proper tissue maturation and cellular differentiation is an indication of appropriate healing from hyperoxia damage.

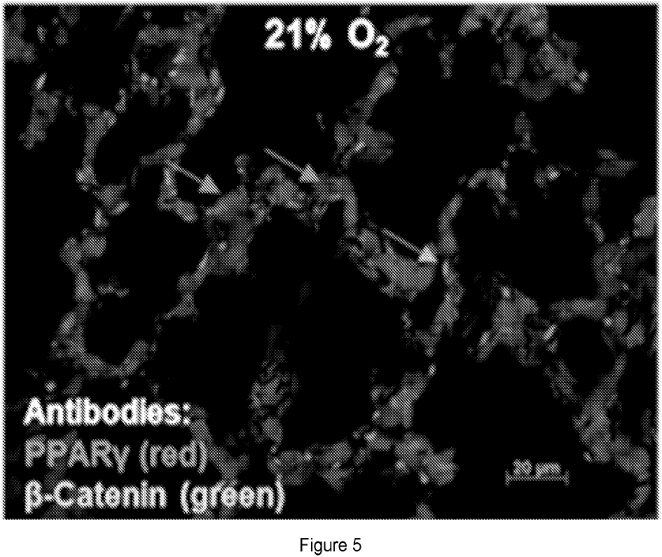

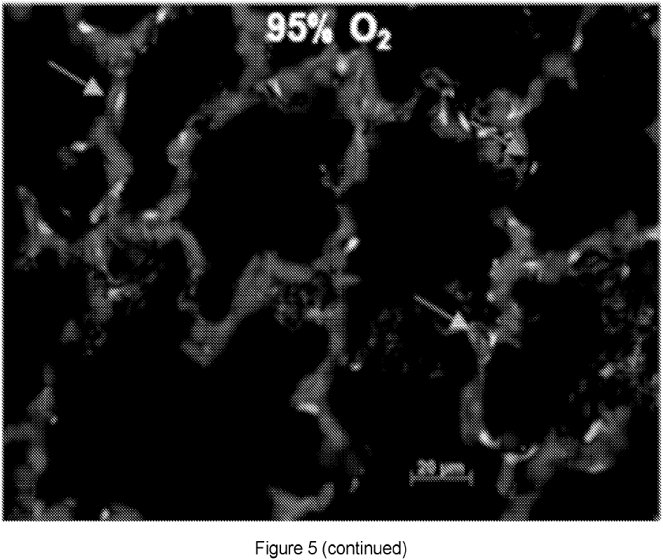

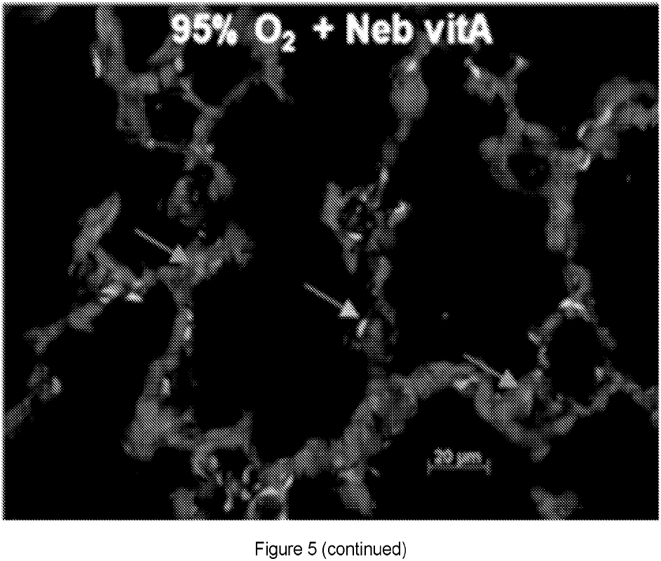

Comparison of the FIGS. 3A, 3B, 4A, and 4B shows the simultaneous but opposite trends: hyperoxia suppresses the level of maturation biomarkers while also stimulating the level of proteins involved in damage-response pathways. Both trends are restored toward the healthy normal condition, with inhaled vitamin A showing an unexpectedly superior potency in each case. Two-color immunofluorescence staining of lung tissue (FIG. 5) provides visual evidence of these simultaneous yet opposite effects.

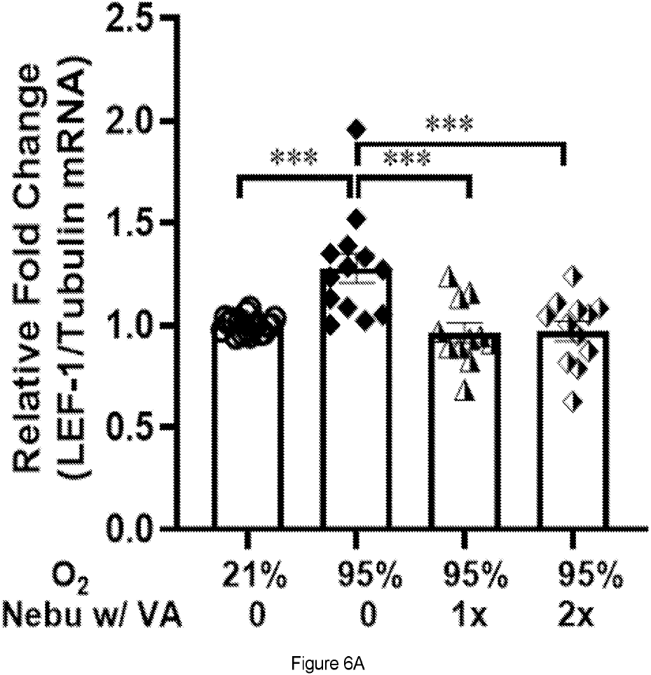

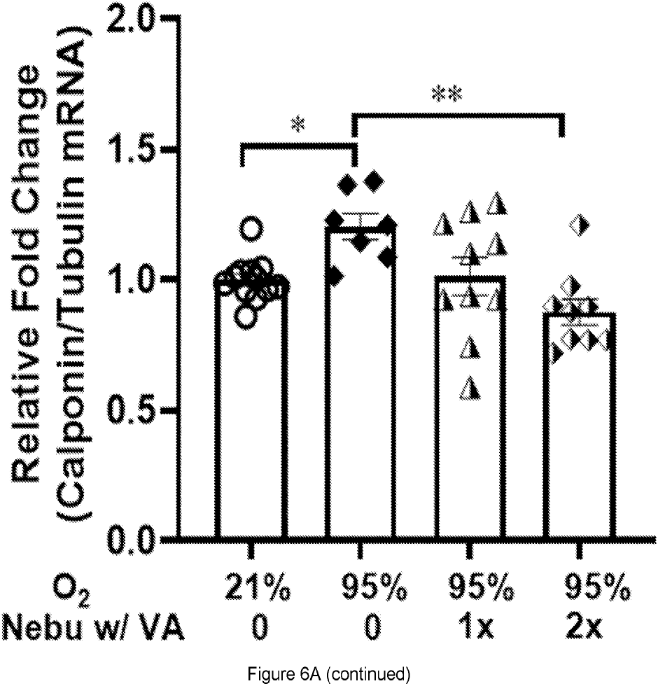

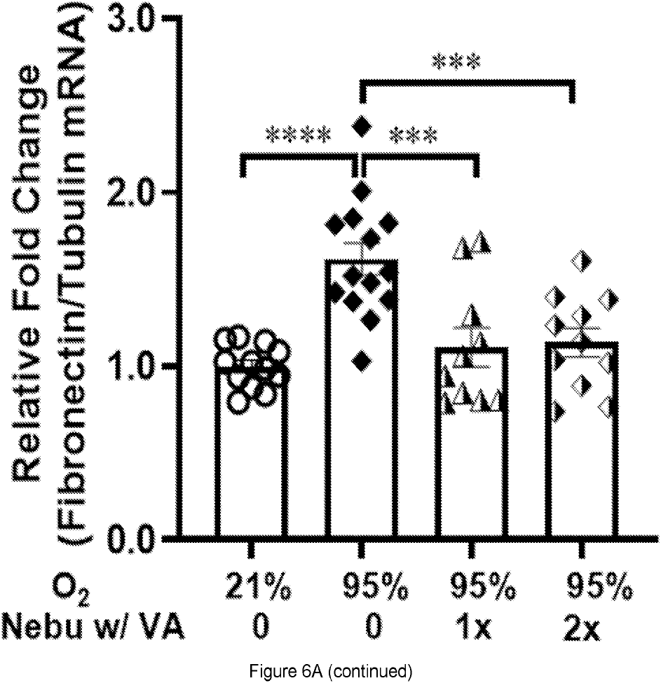

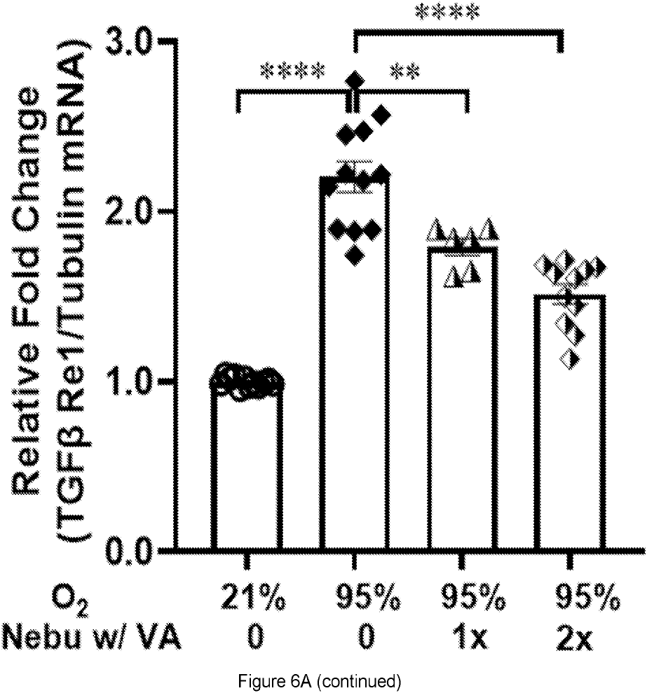

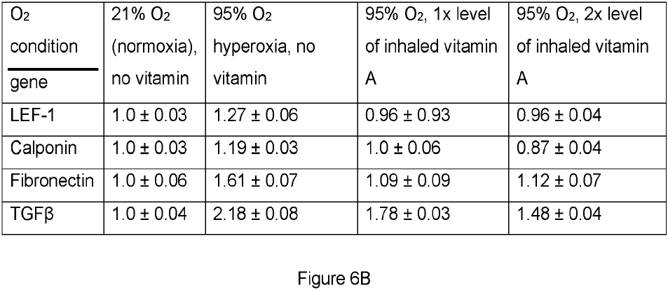

Further evidence of suppression of fibrosis, which could lead to scarring that would impede lung healing and pulmonary function, comes from analysis of expression level of genes related to fibrosis (FIGS. 6A and 6B). Data for 4 genes is shown: LEF1 is known to interact with β-catenin as part of wnt pathway activity while calponin, fibronectin and transforming growth factor beta (TGFβ) play critical roles in cytoskeletal structure and are directly implicated in fibrosis. In fact, Yue et al (Curr Enzym Inhib. 2010 Jul 1; 6(2) described TGFβ as “the titan of lung fibrosis”. The gene expression analyses show increases under untreated hyperoxia conditions but, unexpectedly, restoration to nearly healthy levels when hyperoxia is treated with inhaled vitamin A. The data disclosed also show dose-dependent responses for some of the genes. In this experiment, two different dose levels of vitamin A were attempted in separate animal groups, with dose ‘1x’ indicating the typical dose described above and ‘2x’ reflecting double the nebulized vitamin A dose (with otherwise same dosing schedule and method). TGFβ and, to a lesser extent, calponin show increased response with the increased vitamin A doses. Beyond fibrosis, TGFβ plays a complex role in cell and tissue maturation, so, like the description for neutrophils above, some elevation in TGFβ above healthy normal levels may be beneficial and/or necessary to support the repair of tissue damage caused by the hyperoxia condition itself, thus the observed data can be fully consistent with morphology and other data shown in other Examples.

### Example 1d: Suppression by Inhalation of Vitamin A of Neutrophil Infiltration Induced by Hyperoxia Damage

It is well-known that hyperoxia can induce damage to the microvasculature of the lung, leading to neutrophil infiltration into lung structures such as the alveoli (for example, Li LF, Lee CS, Liu YY, et al. Activation of Src-dependent Smad3 signaling mediates the neutrophilic inflammation and oxidative stress in hyperoxia-augmented ventilator-induced lung injury. Respir Res. 2015;16(1):112.). Quantitation of neutrophils in the alveoli is a method often used to report the extent of lung tissue damage. Beneficial therapies or prophylactic treatments will reduce the neutrophil count relative to levels resulting from untreated hyperoxia damage.

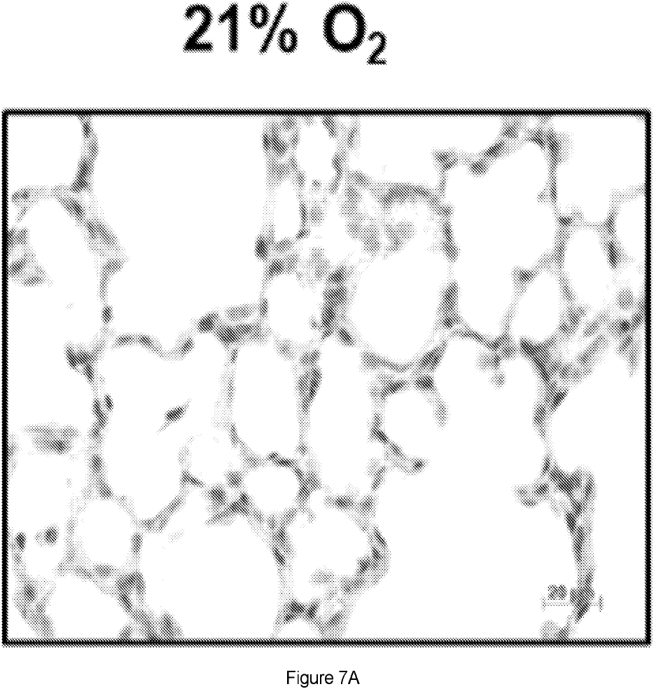

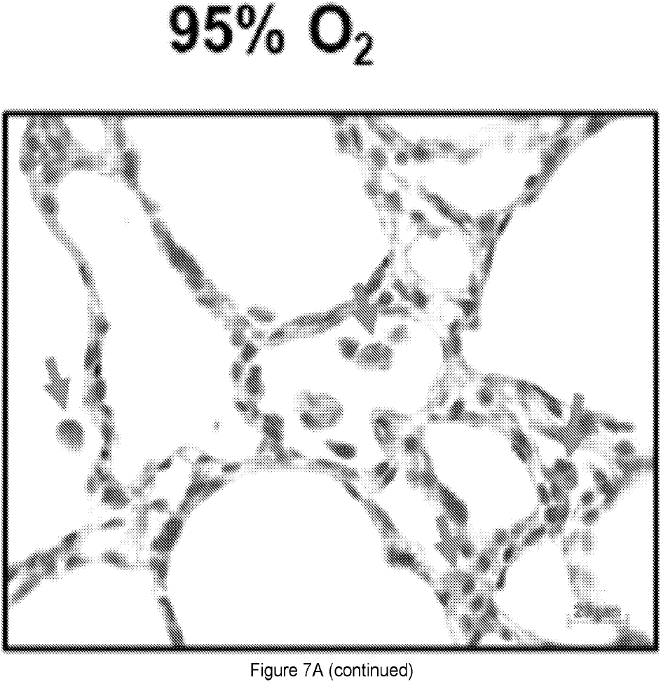

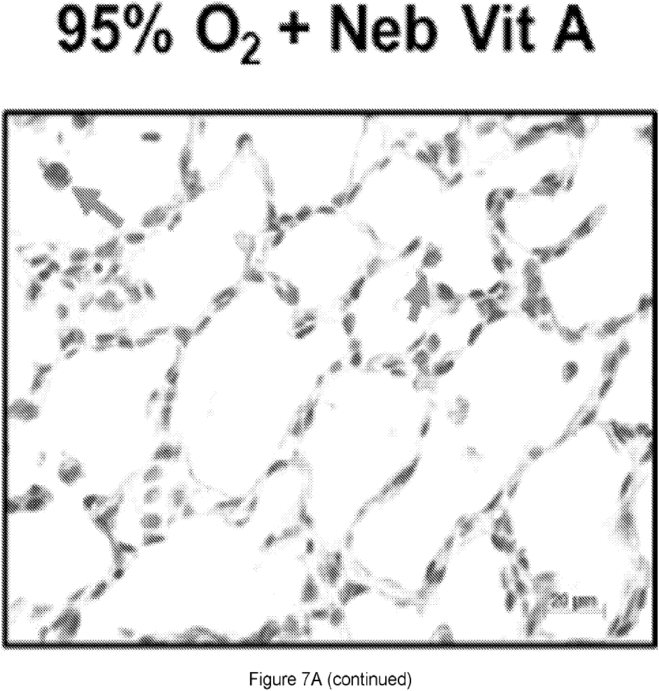

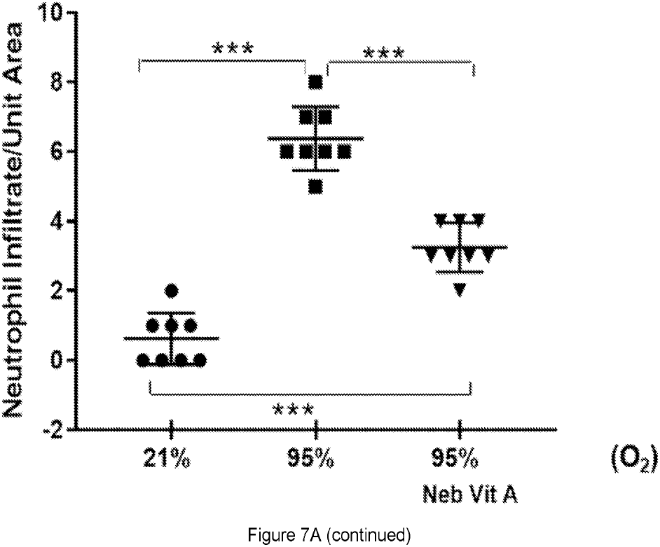

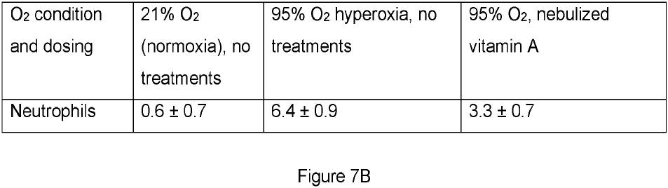

FIGS. 7A and 7B shows that neutrophil count is reduced by approximately 50% when inhaled vitamin A treatment is introduced concurrent with hyperoxia versus untreated hyperoxia. Representative images of contrast-stained alveoli are shown; staining with antibodies against myeloperoxidase are used to specifically identify neutrophils. Quantitation of 8 samples of each group (normal/healthy, untreated hyperoxia and hyperoxia treated with vitamin A by inhalation) is also shown.

In these images, the outlines of alveoli are again depicted, showing the same evidence of nearly normal alveolar structure results from inhaled vitamin A. This latter finding again demonstrates the unexpected result that the morphology of the developing alveoli is unexpectedly maintained in an essentially normal health state with inhaled vitamin A doses, and that the reduced response of the immune system (lower neutrophil count) does not impede maintenance of the structural physiology or developmental status.

### Example 1e : Effect of Vitamin A Dosing Under Normal Oxygen Conditions

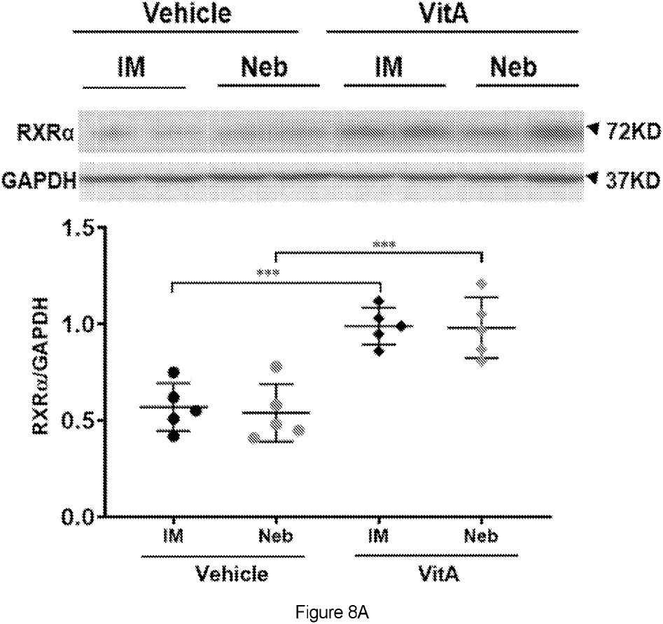

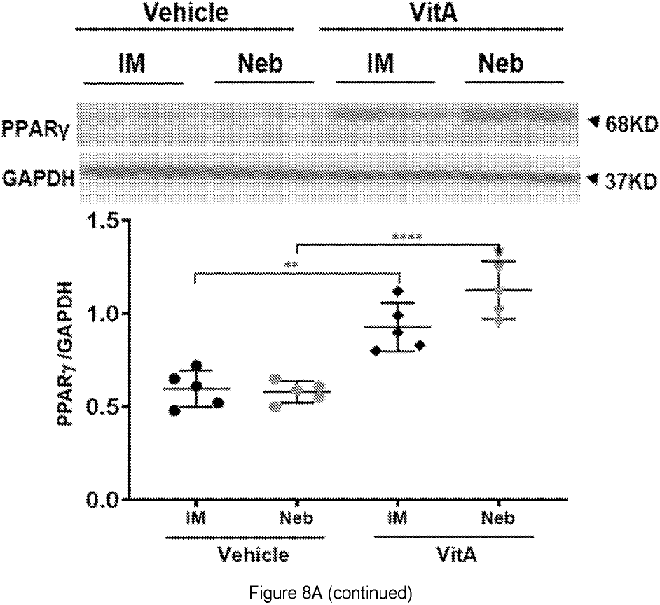

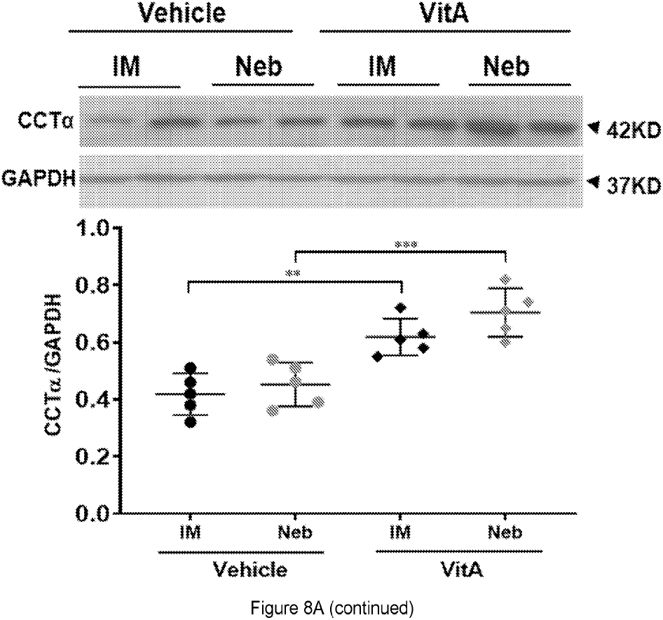

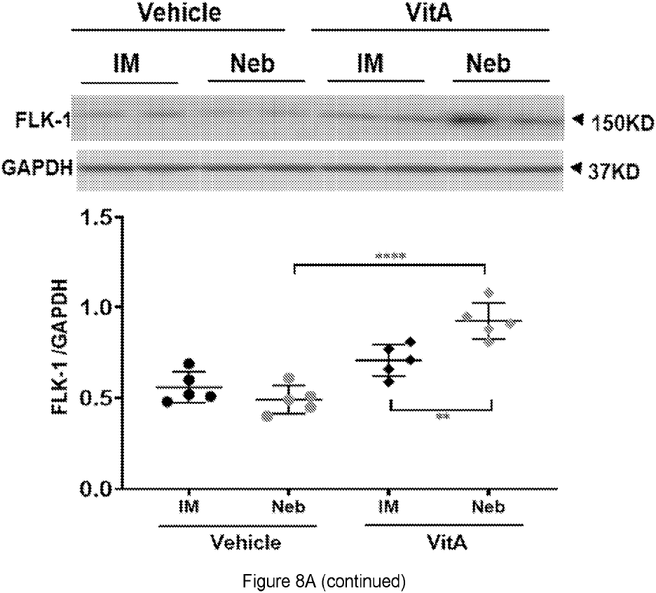

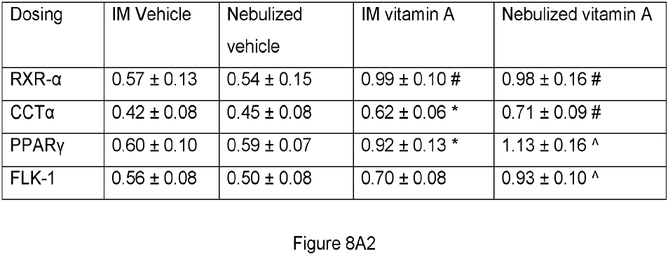

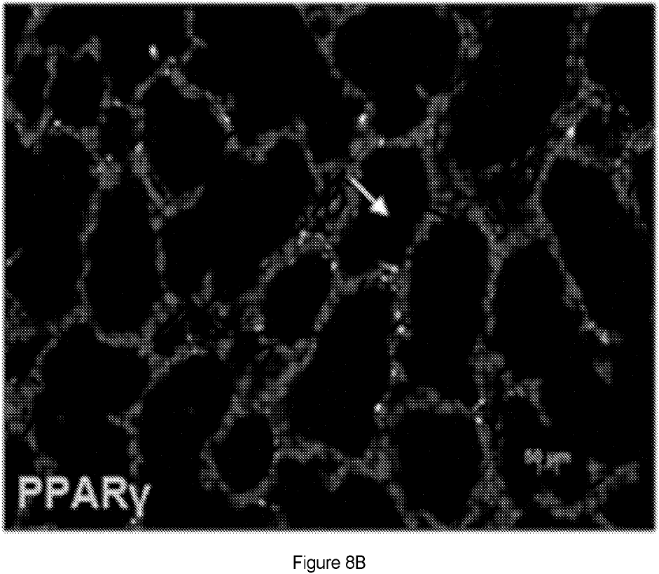

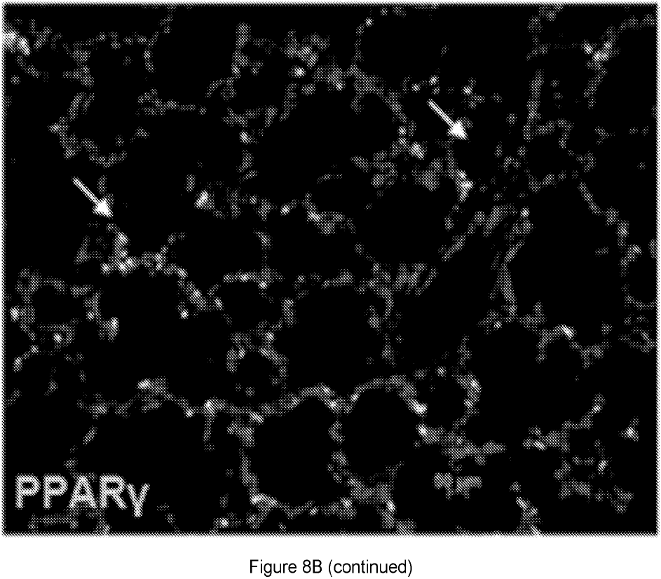

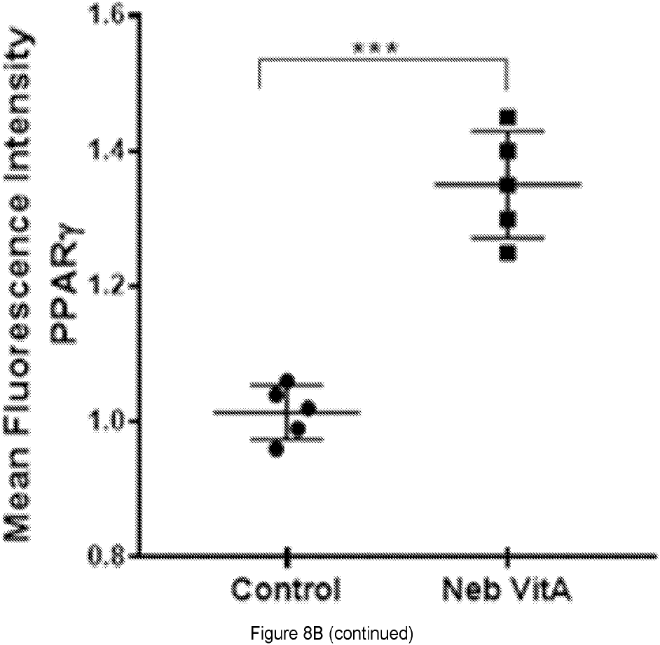

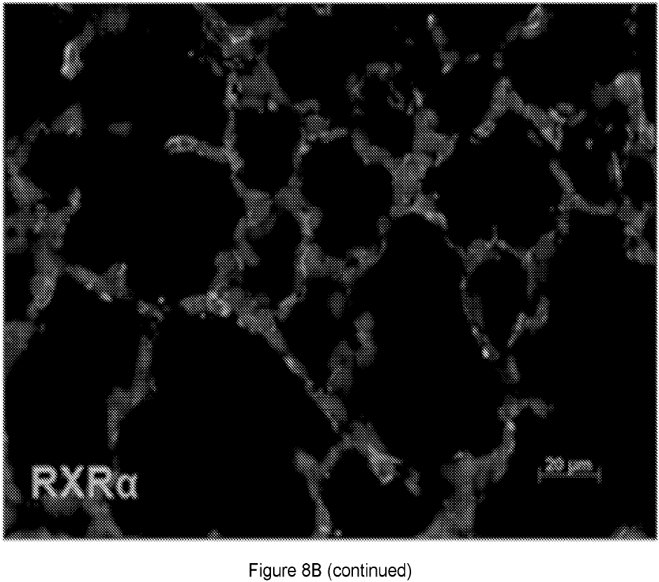

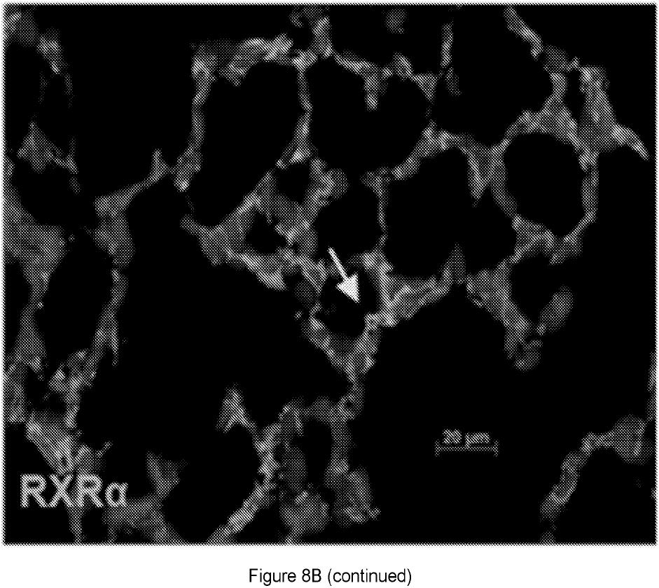

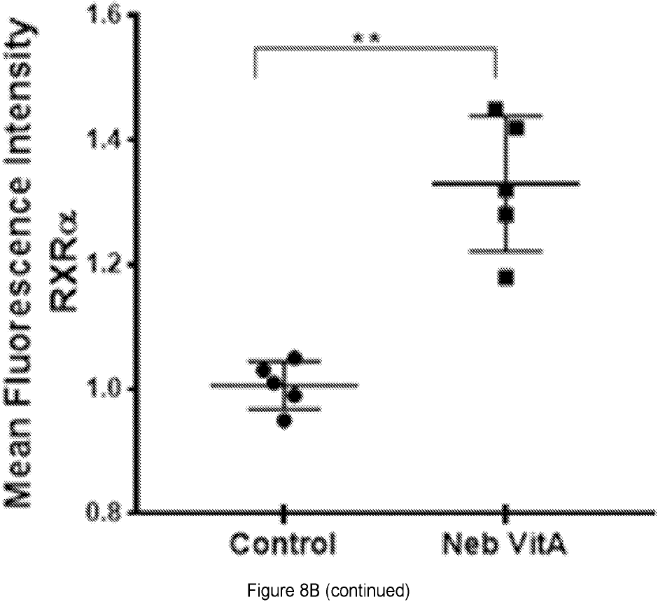

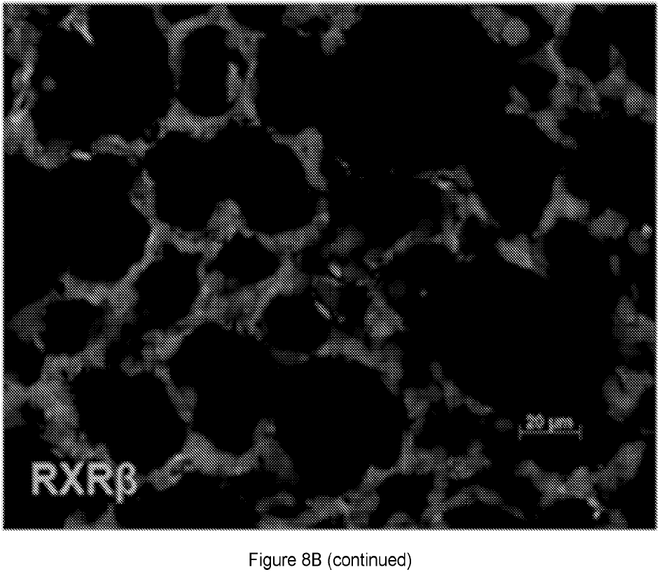

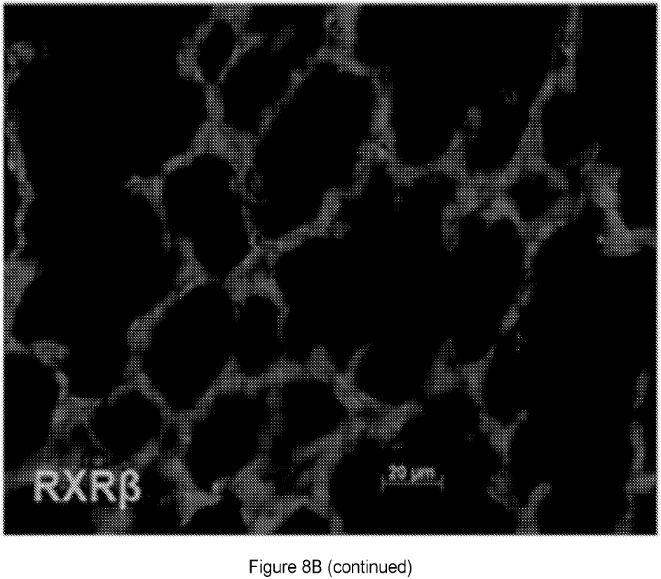

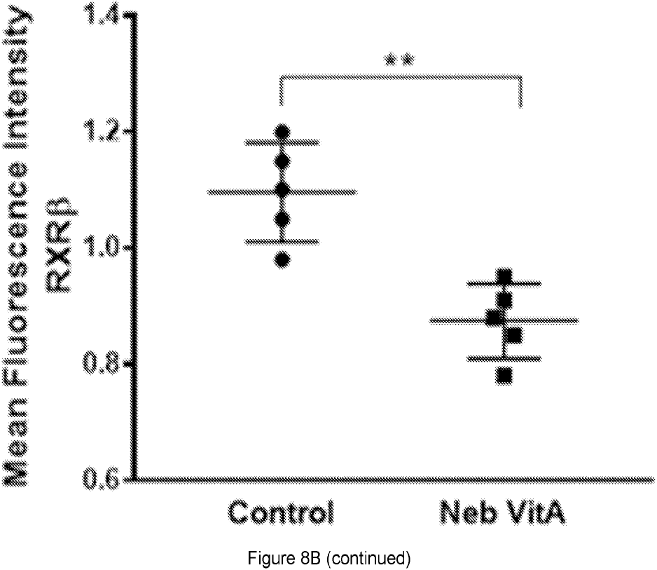

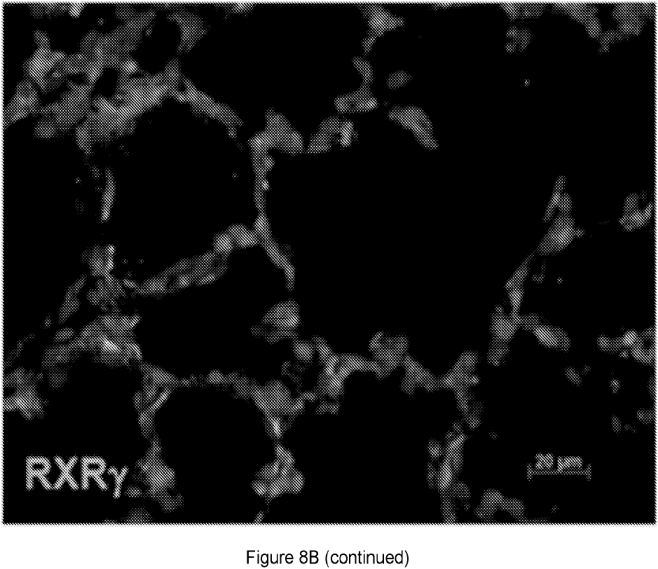

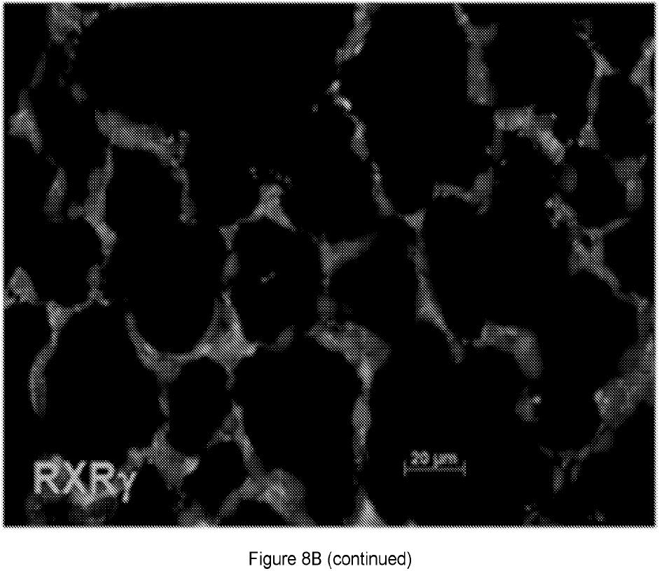

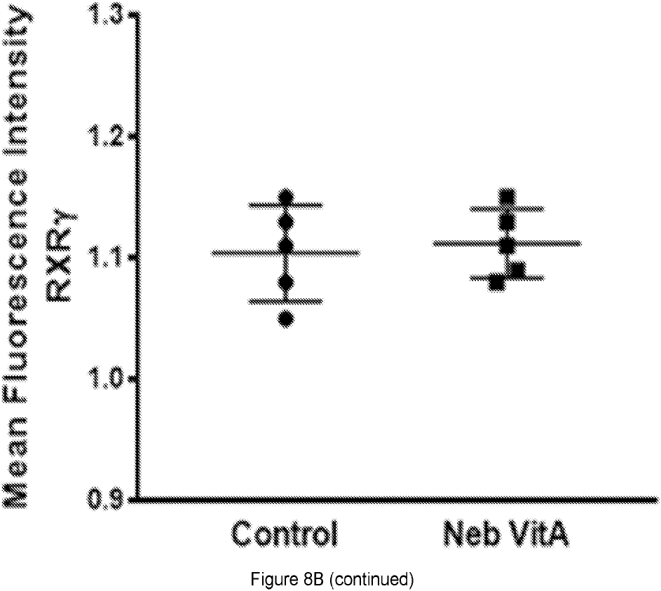

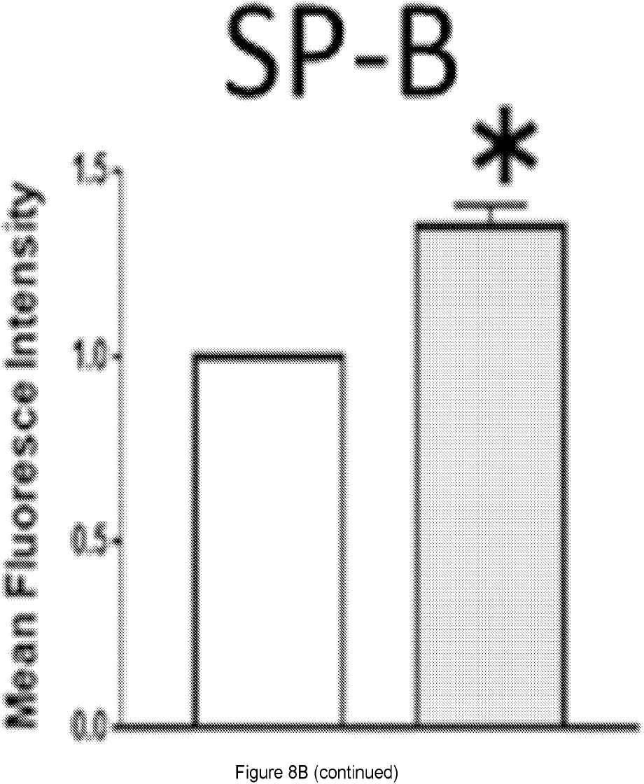

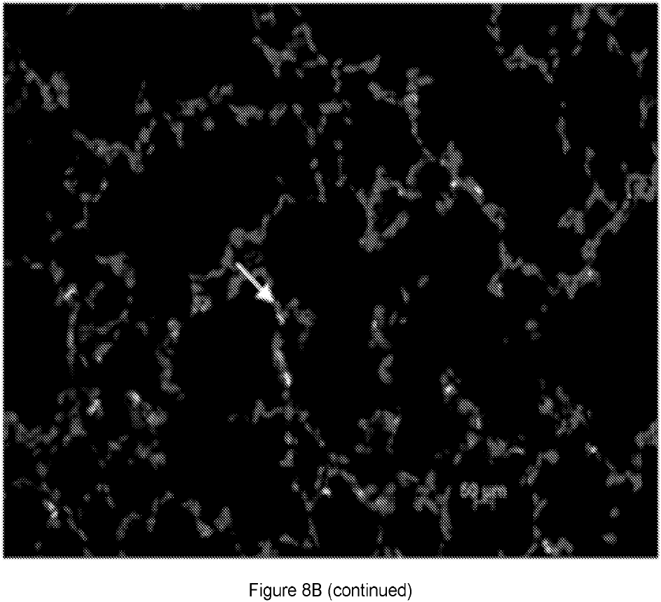

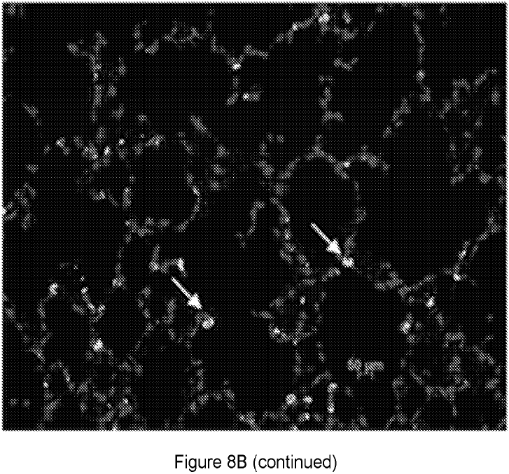

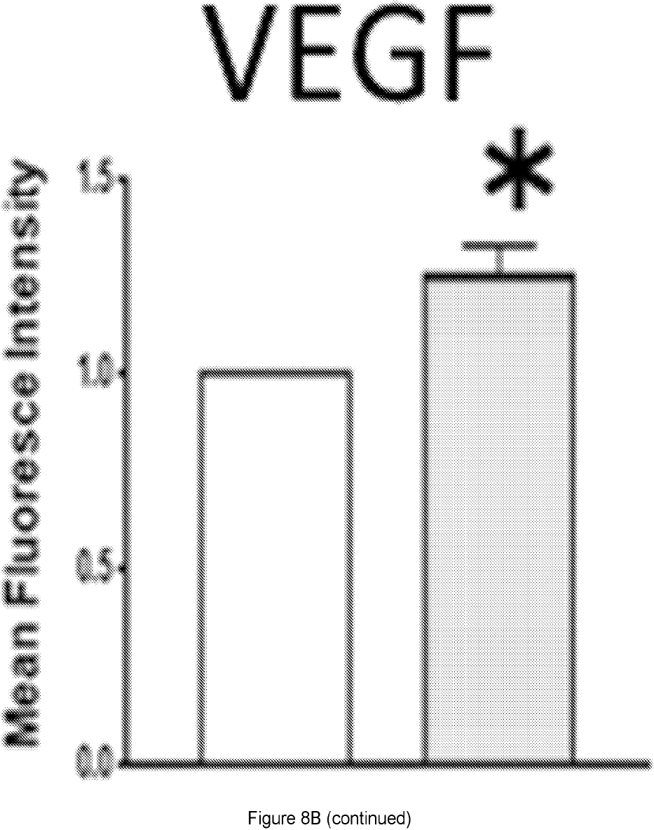

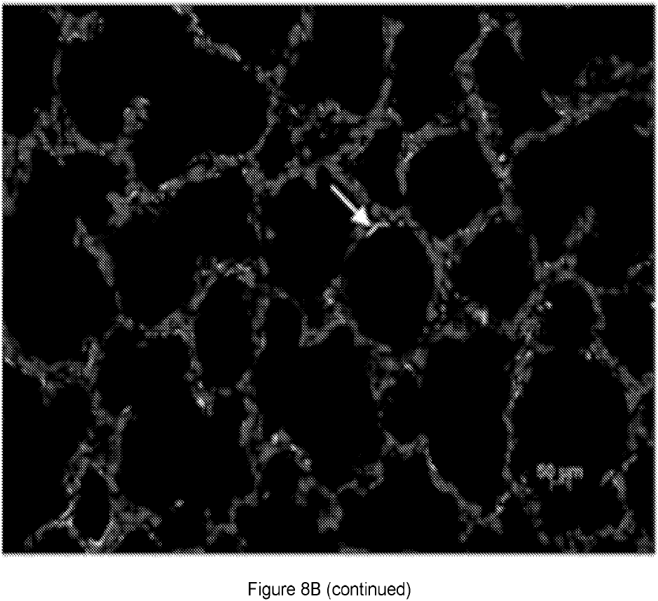

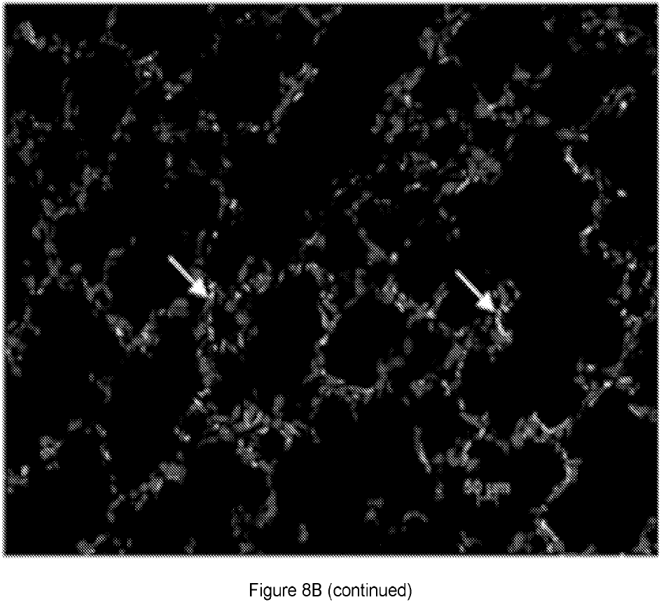

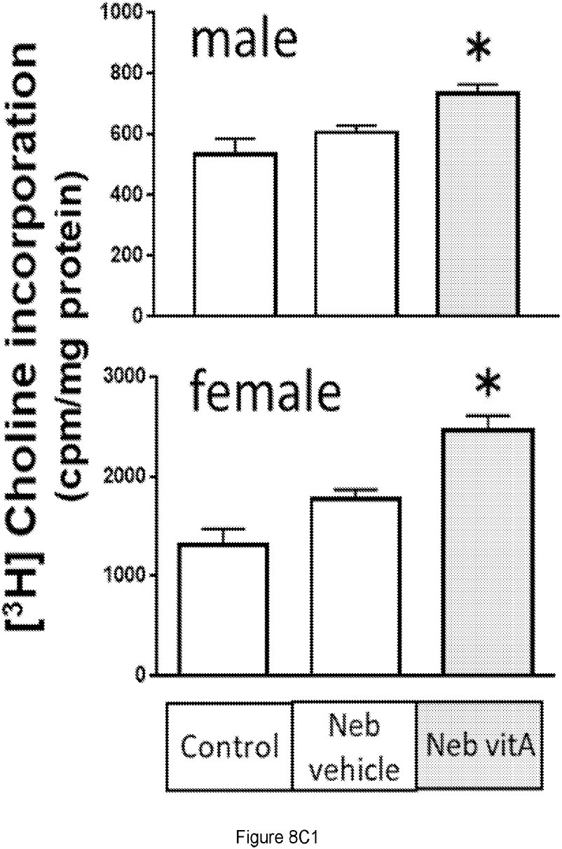

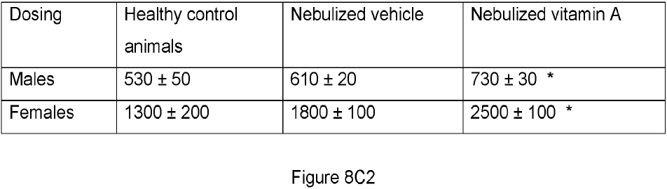

Separate control experiments were performed to examine the effect of the two dosing methods upon the lung under normoxia conditions (FIGS. 8A1, 8A2, 8B, 8C1, and 8C2), with a focus on biomarkers of lung tissue maturation. Both dosing routes result in responses under normxoia, although the inhaled dosing continues to elicit an unexpectedly stronger response than IM. Data support the premise that IM dosing, mimicking current NICU utility, can stimulate lung response, so the dose-route differences evident under hyperoxia cannot be explained merely by some sort of failure of IM dose route itself.

Protein expression levels that change, consistent with stimulation of lung maturation and/or tissue repair, upon vitamin A dosing include the Retinoid X Receptor (RXR) family of receptors of retinoic acid, peroxisome proliferator-activated receptor gamma (PPAR-gamma) increasing, vascular endothelial growth factor (VEGF) increasing, lung surfactant proteins SP-B and SP-c increasing, CTP:phosphocholine cytidylyltransferase subunit alpha (CCT-alpha) increasing, fetal liver kinase 1 (FLK-1) increasing, and also de-novo synthesis of phospholipids as reported by increase in uptake of radiolabeled choline. Data for the RXR family reflects the complex nature of RXR-mediated signaling across a range of pathways, such that RXRα and β responses trend in opposite directions upon vitamin A dosing, while in this case RXRγ is unresponsive; and clearly not all RXR subunits are required to contribute to the overall vitamin A response in lung. However, the data for the RXR family in no way denigrates the data for PPAR-gamma VEGF, SP-B, SP-c, CCT-alpha, FLK-1, and also de-novo synthesis of phospholipids as reported by increase in uptake of radiolabeled choline, all of which indicate an unexpectedly stronger response by inhalation than that for intramuscular administration.

Inhaled vitamin A dosing significantly mitigates harsh hyperoxia-induced lung tissue damage. Compared to IM dosing, inhaled vitamin A dosing unexpectedly results in nearly healthy lung parameters, as judged by morphology (FIGS. 1A, 1B, & 2) and biomarkers representative of lung maturation and damage-repair pathways (FIGS. 3-8). The levels of the effect of vitamin A dosing can be judged against untreated (vehicle-treated) normoxic and hyperoxic control groups. Importantly, the harsh hyperoxia condition used here (95% O₂ for 7 days) represents an aggressive biological insult, far exceeding typical NICU oxygen levels, so the extent to which particularly the inhaled vitamin A dosing mitigates hyperoxic lung damage is strongly supportive of future clinical utility for inhaled vitamin A dosing for human neonates. One critical characteristic of this study is that the hyperoxia and vitamin A dosing are simultaneous, a study design that directly assesses vitamin A ability to ‘mitigate’ or ‘prevent’ hyperoxia-induced damage, in this case to developing neonatal lung tissue.

Comparatively weak IM dose-response. It is unexpected and surprising that the IM dose, designed to mimic current NICU vitamin A utilization, elicits a far weaker response than the inhaled vitamin A dosing. Data indicate that IM dosing indeed does have a response, but only a subset of the biomarkers analyzed indicate that IM dosing differs significantly from untreated hyperoxia, with just one instance among our tests (SP-C) of a biomarker for which the IM-dosed hyperoxia condition achieves a nearly healthy (normoxia control) level. A similar study on animals under normal oxygen (FIGS. 8A1, 8A2, 8B, 8C1, and 8C2) validates that IM dosing is viable for eliciting lung responses, showing stronger responses than IM doses induce during hyperoxia, but is still often weaker than the response to inhaled doses. Although muted under normoxia, the unexpected superiority in response to the inhaled dose versus injected dosing is still present. There was no a priori expectation of this difference of response under hyperoxia vs normoxia, particularly for the IM dosing since it is the standard of care in NICU settings.

Superior response to inhaled dosing reflects a potential direct-to-target-organ advantage over typical IM dosing. The direct-to-target-organ advantage of inhaled vitamin A dosing was a core hypothesis at the outset, with specific focus on the NICU use of vitamin A to prevent BPD, a lung condition that might logically benefit from direct lung exposure to an appropriate inhaled therapy although before the present disclosure the logical benefit of inhaled vitamin A doses was theoretical and the actual medical benefit needed to be demonstrated, given the unpredictable behavior of any drug formulation in vivo. However, the results for inhaled vitamin A disclosed herein unexpectedly exceed the results that would have been expected for inhaled vitamin A. Rather, a more reasonable a priori expectation was that inhaled doses would result in medical outcomes more similar to those achieved by IM vitamin A doses, particularly in light of the severe hyperoxia condition used in this model system (95% oxygen) which far exceeds any level that would be used medically because of the known damage that oxygen can cause to lung tissue.. Efficacious inhaled dosing would also be non-invasive, overcoming a variety of known hurdles of reliance on injection (Green, J., et al., It’s agony for us as well: Neonatal nurses reflect on iatrogenic pain. Nurs Ethics. 2016:23(2):176-90. PMID: 254887861; and Services D of H&H. Intramuscular injections for neonates [Internet] Available from: Https://www2.health.vic.gov.au:443/hospitals-and-health-services/patient-care/perinatal-reproductive/neonatal-handbook/procesures/intramuscular-injections). Indeed, especially with the hyperoxia data, this hypothesis is supported by the unexpectedly stronger lung responses to the inhaled dose, resulting in many metrics achieving nearly healthy lung status despite the intentionally harsh hyperoxia condition. Inhalation dosing of the disclosed pharmaceutical compositions represents a simultaneously non-invasive and more efficacious use of vitamin A to prevent neonatal hyperoxia lung damage, and presumably to prevent BPD clinically, which may lead to important clinical benefits upon further development.

The apparent extent of the inhalation advantage under hyperoxia was unexpected. An unexpectedly powerful response to the inhaled doses results in nearly healthy lung according to many of the metrics described herein, compared to the surprisingly weaker response to IM dosing. Nor was the extent to which hyperoxia apparently suppresses the utility of IM vitamin A dosing compared to the more vigorous response to IM dosing under normoxia, expected. While it is plausible that a presumed 20% inhalation dose-delivery efficiency in the current whole-body exposure system may be an overestimate, that possibility actually would accentuate the apparent superiority of the inhalation dose vs IM (e.g. an unexpectedly potent efficacy from a potentially an even lower amount of inhaled vitamin A).

Inhaled vitamin A dosing efficacy seems to be based on at least two distinct mechanisms of action in treating hyperoxia lung damage, as follows: 1) support of healthy lung maturation processes despite concurrent severe hyperoxia damage; and 2) stimulation of sufficient healing/repair processes against the hyperoxia-induced damage while avoiding deleterious, excessive, and/or shock-like repair responses. Together, these outcomes result in lung tissue morphology being maintained in state very close to healthy normal tissue.

### Example 2 - Inhalation Dosing for Treating Vitamin A Deficiency

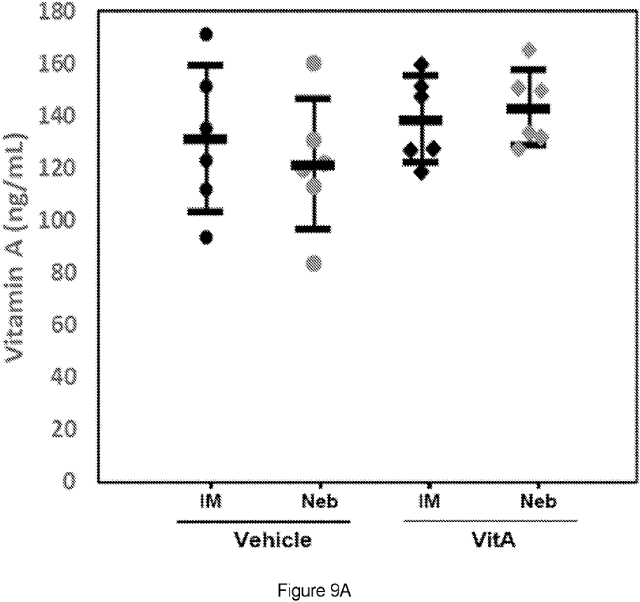

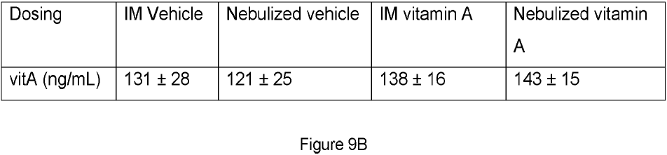

As disclosed herein, inhaled vitamin A is an effective treatment for vitamin A deficiency. Inhaled vitamin A treatment raised the level of vitamin A in circulation, as reflected in elevated levels in serum after 7 days of alternate day (days 1, 3, 5, 7) inhalation dosing of neonatal rats. A study used 4 groups of animals: two groups dosed with vitamin A, via either inhalation or intramuscular (IM) injection, and two dosed with vitamin-A-free vehicle solution as controls, also either via inhalation or injection. After 7 days, serum was collected and tested for vitamin A level via ELISA. Both vitamin-A dosing methods resulted in an increase in serum vitamin A level compared to the vehicle-dosed control groups (FIGS. 9A and 9B), and the level achieved is the same, suggesting that the inhaled vitamin is unexpectedly equally capable of elevating systemic (serum) levels of vitamin A as traditional injection dosing. This result was unexpected in view of the very different methods of delivery. Importantly, no animal groups were vitamin A deficient, and all were feeding normally throughout the study, so the broad range of vitamin A in the vehicle-dosed groups reflects the normal healthy serum vitamin A range in these animals. The vitamin A-dosed groups each show serum levels raised to essentially the ‘upper half’ of the range for vehicle-treated controls.

Elevation of serum levels of vitamin A is an important reflection of systemic distribution, as such elevation indicates that the dose becomes available to target organs via the blood circulation. Although no animals were vitamin A deficient in this study, the elevation of serum levels demonstrates the necessary outcome for treating vitamin A deficiency. That inhaled dosing of vitamin A unexpectedly treats lungs more effectively than injected doses, as reflected in the other lung biomarker results in the previous examples, did not intrinsically demonstrate that systemic dosing, presumed to follow dosing by injection, would also happen upon vitamin A inhalation. Therefore, the finding that inhaled vitamin A resulted in elevated serum levels of vitamin A similar to that for IM vitamin A would have been unexpected. Increases in serum levels following inhalation, unexpectedly equivalent to injected dosing, demonstrates that the inhaled dose can equally support body-wide distribution of the dose. Importantly, there is no evidence that vitamin A dosing resulted in significant excess of serum vitamin A compared to healthy animals, suggesting that the dosing protocols used do not introduce hypervitamin-A-osis (or essentially overdose of vitamin A). From the serum-level data, inhalation of vitamin A can now be viewed as a viable overall replacement for injection dosing, representing simultaneously a benefit to lung treatment, as disclosed in the other examples, while also being equivalent for other organ dosing through body-wide distribution in the blood as a treatment for vitamin A deficiency.

The data demonstrate the ability of either dosing route to offset vitamin A deficiency conditions, such as those that have been described to occur during active infections, as disclosed herein. Thus, benefits of vitamin A doses beyond the lung and/or respiratory pathway are unexpectedly achieved.

### Example 3: Inhalation Dosing of Vitamin A for Mitigating Iatrogenic Damage From Hyperoxia

As described above, there are many conditions that can lead to decreased blood oxygen levels, and in many cases required medical intervention includes use of enhanced oxygen levels (hyperoxia) to support lung function and maintain sufficient blood oxygen. Patients often require increased oxygen supply, perhaps receiving oxygen directly by nasal canula, or often severe patients will be intubated so that external respiratory support can be provided. In these cases, lungs are exposed to higher oxygen levels than are typical in normal air. The extra oxygen helps overcome deficiencies in lung function, but comes at the price of inducing hyperoxia damage. It is well known that exposure to oxygen levels in excess of normal air (which is ~21% oxygen) begin to damage the lung, first by degrading the physical properties of the fluid (pulmonary surfactant) that is produced by the lung to maintain the lung’s cellular integrity for the portion of lung exposed to air, and ultimately by tissue damage (particularly tears in the alveolar lining) that can lead to localized bleeding, and representing a risk of any lung-borne infectious agents being able to enter the bloodstream and cause systemic infection or sepsis. Thus, the oxygen provided to support critical lung function also can damage the lung, representing a form of iatrogenic damage.

The model system described in the examples above serves as a model of generalized lung damage typical of hyperoxia, having exposed animals to 7 days of continuous dwell in a 95% oxygen environment, a level of oxygen far exceeding any typical medical use for humans (typically no more than 40% oxygen would be used). The results described herein demonstrate the unexpectedly potent prophylactic effect that inhaled vitamin A doses have for preventing hyperoxia damage to lung tissue. The results reflect several key effects: lung tissue morphology is maintained at levels matching healthy normal lung (FIGS. 1A, 1B, & 2) despite harsh and continuous hyperoxia exposure, and simultaneously that deleterious biochemical pathways (e.g. fibrosis, scarring, shock pathways) have been suppressed. For the former, it was unexpected that inhaled vitamin A doses would be so robust as to result in levels of many of these metrics being at normal healthy levels, particularly because of the extreme hyperoxia conditions used. For the latter, it is also notable that biochemical pathways of tissue repair are maintained at intermediate levels, such that required healing is supported but response is suppressed to avoid levels that might correlate with deleterious effects of an excessive healing response. Data indicative of this ‘beneficially intermediate’ response include neutrophil count (FIGS. 7A and 7B) and certain repair/shock biomarkers (e.g. ALK-5; FIGS. 4A and 4B). Of note, preservation of some neutrophil increase would be particularly advantageous for patients with lung infection and/or iatrogenic damage resulting from treatment of infection, since the neutrophils, part of immune response, are important in usual biological response to infection.

It is important to reiterate that the extreme oxygen level (95%) used in this model system is meant to induce far more damage than the more typical 35-40% oxygen levels used for human in hospital. This model system presents a ‘high bar’ for demonstrating the benefits of the used of inhaled vitamin A. As such, the demonstrable and unexpected benefits of inhaled vitamin A, under this severe insult, are likely to be even more profound when used in conjunction with more typical therapeutic oxygen levels. Additionally, the known upper limit of therapeutic oxygen level might be able to be increased since the hyperoxia damage is currently one factor limiting higher oxygen – offsetting the damage to lung tissue might allow higher therapeutic oxygen (at least for brief periods) that could help the most severe patients maintain sufficient blood oxygenation while undergoing other treatments to improve lung function.

What is claimed is:

1. A pharmaceutical composition comprising an aqueous formulation comprising an insoluble vitamin at least partially solubilized with a surfactant wherein the pharmaceutical composition is in liquid aerosol form.

2. The pharmaceutical composition according to claim 1, wherein the insoluble vitamin comprises at least one of vitamin A, all-trans retinol, 13-cis-retinol, the palmitate ester of all-trans retinol, the acetate ester of all-trans retinol, the maleate ester of all-trans retinol, 9-cis-retinol, 9,13-dicis-retinol, 3,4-didehydroretinol, CD437, CD1530, and an intrinsically fluorescent retinoid analog.

3. The pharmaceutical composition according to claim 1, wherein the insoluble vitamin comprises at least one of all-trans retinol, the palmitate ester of all-trans retinol, the acetate ester of all-trans retinol, the maleate ester of all-trans retinol, wherein the surfactant comprises polysorbate 80, and wherein the pH of the pharmaceutical composition is 7.0 to 7.5.

4. The pharmaceutical composition according to claim 3, comprising up to 6.0% (w/w) of the all-trans retinol, the palmitate ester of all-trans retinol, the acetate ester of all-trans retinol, the maleate ester of all-trans retinol, and/or a mixture thereof.

5. The pharmaceutical composition according to claim 1, wherein a weight of the surfactant is at least 3.0 times the weight of the insoluble vitamin.

6. (canceled)

7. (canceled)

8. The pharmaceutical composition according to claim 1, wherein the insoluble vitamin comprises at least one selected from the group consisting of all-trans-retinol, 13-cis-retinol, 9-cis-retinol, 9,13-dicis-retinol, 3,4-didehydroretinol, the, palmitate ester of all-trans-retinol, the acetate ester of all-trans-retinol, the maleate ester of all-trans-retinol, CD437, CD1530, an intrinsically fluorescent retinoid analog and/or a mixture thereof, wherein a weight of the surfactant is 3.0 times and 8.5 times the weight of the all-trans-retinol, 13-cis-retinol, 9-cis-retinol, 9,13-dicis-retinol, 3,4-didehydroretinol, rthe the maleate ester of all-trans-retinol, 3,4-didehydroretinol, CD437, CD-1530, an intrinsically fluorescent retinoid analog, and mixtures thereof, and, optionally, wherein the pharmaceutical composition further comprises a conjugate base of a pharmaceutically acceptable acid and/or a conjugate acid of a pharmaceutically acceptable base.

9-12. (canceled)

13. The pharmaceutical composition according to claim 1, wherein the surfactant comprises polysorbate 20, polysorbate 60, polysorbate 80, stearyl alcohol, sorbitan monolaurate, sorbitan monopalmitate, sorbitan monostearate, polyoxyethylene (20) oleyl ether, polyoxyethylene (20) cetyl ether, polyoxyethylene (10) cetyl ether, polyoxyethylene (10) oleyl ether, polyoxyethylene (100) stearyl ether, polyoxyethylene (10) stearyl ether, polyoxyethylene (20) stearyl ether, polyoxyethylene (4) lauryl ether, polyoxyethylene (20) cetyl ether, polyoxyethylene (2) cetyl ether, caprylocaproyl polyoxyl-8 glyceride, polyethylene glycol (20) stearate, polyethylene glycol (40) stearate, polyethylene glycol, polyethylene glycol (8) stearate, polyoxyl 40 stearate, poloxamer 188, polaxamer 312, or mixtures thereof.

14. The pharmaceutical composition according to claim 1, wherein the surfactant comprises polysorbate 80.

15-20. (canceled)

21. The pharmaceutical composition according to claim 1, wherein the surfactant comprises polysorbate 80 and the fatty acid content of the polysorbate 80 is between 85% and 100% oleic acid.

22-26. (canceled)

27. The pharmaceutical composition according to claim 1, wherein the pharmaceutical composition further comprises:
at least one antibiotic compound comprising at least one penicillin, at least one tetracycline, at least one cephalosporin, at least one quinolone, at least one lincomycin, at least one macrolide, at least one sulphonamide, at least one glycopeptide, at least one amino glycoside, a carbapenem, and/or a mixture thereof;
at least one insoluble vitamin comprising Vitamin D, an analog of Vitamin D, a derivative of Vitamin D, Vitamin E, an analog of Vitamin D, a derivative of Vitamin D, Vitamin K, an analog of Vitamin K, a derivative of Vitamin K, Vitamin B6, niacinamide, Vitamin B2, Vitamin B1. dexpanthenol, biotin, folic acid, vitamin B12, and/or a mixture thereof;
an anti-viral compound comprising oseltamivir, zanamivir, peramivir, ribavirin, remdesivir, at least one nucleoside analog, at least one interferon, at least one protease inhibitor, at least one reverse transcriptase inhibitor, at least one neuramidinidase inhibitor, and/or a mixture thereof; and/or
a sugar comprising glucose, arabinose, maltose, sucrose, dextrose, lactose, and/or a mixture thereof; and/or
caffeine, epinephrine, and/or a mixture thereof.

28-39. (canceled)

40. The pharmaceutical composition according to claim 1, wherein the pharmaceutical composition does not comprise a preservative.

41-50. (canceled)

51. A pharmaceutical composition comprising an aqueously insoluble vitamin that has been at least partially solubilized with a surfactant wherein the pharmaceutical composition is in liquid aerosol form.

52-57. (canceled)

58. A pharmaceutical composition comprising:
i) vitamin A, or a pharmaceutically acceptable isomer, analog, and/or derivative thereof, or a mixture thereof;
ii) a surfactant, and
wherein the pharmaceutical composition is in aerosol form.

59. The pharmaceutical composition according to claim 58, wherein the surfactant comprises a non-natural surfactant.

60-64. (canceled)

65. The pharmaceutical composition according to claim 58, wherein the pharmaceutical composition is a liquid and wherein the vitamin A, or a pharmaceutically acceptable isomer, analog, and/or derivative thereof, or a mixture thereof, is disposed within a micelle.

66-78. (canceled)

79. AThe pharmaceutical composition according to claim 58, wherein the pharmaceutical composition consisting essentially of vitamin at A, or a pharmaceutically acceptable isomer, analog, and/or derivative thereof, or a mixture thereof, and a surfactant wherein.

80. A system for use in the prophylaxis or treatment of a vitamin deficiency disorder, hyperoxia damage, oxidative lung injury (lung damage), lung damage caused by chemical or environmental exposure, lung damage caused by a biological/infectious agent, lung damage caused by a chemical, biological, or radio-nuclear (CBRN) agent, and lung damage caused by radio-nuclear exposure, wherein the system comprises a therapeutically effective dose of a pharmaceutical composition according to claim 1, and a device suitable for generating the liquid aerosol form thereby forming a plurality of liquid droplets wherein the liquid droplets produced by the system have a mass median aerodynamic diameter of less than 15 microns.

81. (canceled)

82. The system according to claim 13, wherein the liquid droplets have a mass median aerodynamic diameter of less than 5 microns.

83-87. (canceled)

88. The pharmaceutical composition according to claim 1, for use in the treatment or prophylaxis in a patient in need thereof of a vitamin deficiency disorder, hyperoxia damage, lung damage caused by oxidative lung injury, lung damage caused by chemical or environmental exposure, lung damage caused by a biological/infectious agent, lung damage caused by a chemical, biological, or radio-nuclear (CBRN) agent, lung damage caused by radio-nuclear bronchopulmonary dysplasia, retinopathy of prematurity, neonatal sepsis, hypovitaminosis A, hospital-acquired sepsis, sepsis from premature rupture of membranes, meningitis, pneumonia, necrotizing enterocolitis, radiation-induced pneumonitis, a viral infection, and/or a bacterial infection.

89-130. (canceled)

131. The pharmaceutical composition according to claim 1, for use in supporting a patient’s immune response in improving the efficacy of a vaccine against an infectious agent in a patient.

132-195. (canceled)
